(19) United States
(12) Patent Application Publication
(10) Pub. No.: US 2004/0254363 A1
(43) Pub. Date: Dec. 16, 2004

(54) Genes and snps associated with eating disorders

(76) Inventors: Andrew Bergen, CabinJohn, MD (US); Meredith Yeager, Dickerson, MD (US)

Correspondence Address: MORGAN LEWIS & BOCKIUS LLP, 1111 PENNSYLVANIA AVENUE NW, WASHINGTON, DC, 20004, US

(21) Appl. No.: 10/483,958

(22) PCT Filed: Jul. 16, 2002
(86) PCT No.: PCT/US02/22555

Related U.S. Application Data
(60) Provisional application No. 60/305,153, filed on Jul. 16, 2001.
(60) Provisional application No. 60/306,440, filed on Jul. 20, 2001.
(60) Provisional application No. 60/331,285, filed on Nov. 13, 2001.
(60) Provisional application No. 60/340,843, filed on Dec. 19, 2001.
(60) Provisional application No. 60/340,844, filed on Dec. 19, 2001.

(51) Int. Cl. C12Q 1/68; C07H 21/04

(52) U.S. Cl. 536/23.2; 435/6

(57) ABSTRACT

The invention relates generally to polymorphisms in the serotonin receptor 1D gene, the delta-opioid receptor gene, and the dopamine receptor D2 gene that are associated with eating disorders such as anorexia nervosa and bulimia nervosa. The invention also relates to composition screening systems and diagnostic and prognostic assays for eating disorders.

### RELATED APPLICATIONS

The present application claims priority to U.S. Provisional Patent Applications 60/305,153; 60/306,440; 60/331,285; 60/340,843; and 60/340,844, all of which are herein incorporated by reference in their entirety.

### FIELD OF THE INVENTION

The invention relates generally to the association of genes and single nucleotide polymorphisms (SNPs) with eating disorders such as anorexia nervosa and bulimia. The invention relates specifically to the discovery of polymorphisms in the HTR1D, OPRD1, DRD2, and other genes and the association and linkage of these polymorphisms with an eating disorder such as anorexia nervosa or bulimia nervosa.

### BACKGROUND OF THE INVENTION

A variety of life-threatening feeding and energy homeostasis disorders have been recognized in the medical literature. Such disorders include, for example, the eating disorders anorexia nervosa (AN) and bulimia nervosa (BN), as well as obesity. AN and BN are severe psychiatric illnesses with significant morbidity and mortality that affect approximately 3% of women. In addition to weight loss, AN patients may also suffer from cachexia, cardiac dysfunction, leukopenia, osteoporosis and a variety of gastrointestinal and neuropsychiatric conditions. See, e.g., Walling (2000), American Family Physician 8: 2528. In addition, AN patients typically have low self-esteem and are known to have obsessive tendencies in some cases.

AN (˜0.5% prevalence) may be defined and diagnosed by the following psychiatric criteria: refusal to maintain weight, fear of gaining weight, and a disturbance in the patient's perception of body weight or shape and its effect on self-evaluation. BN (˜2.5% prevalence) may be defined and diagnosed by the following psychiatric criteria: regular episodes of binge eating, a sense of lack of control during the binge episode, inappropriate compensatory behavior (e.g., purging) to avoid weight gain, and a disturbance in the patient's self-evaluation due to perceived body shape and weight (American Psychiatric Association (1994) Diagnostic and Statistical Manual of Mental Disorders, American Psychiatric Association, Washington, D.C.). Other eating disorders include pica (eating inappropriate or non-nutritive substances such as clay, paint or ice) and rumination (repeated regurgitation of food, usually occurring in infants).

Comorbidity of eating disorders and other psychiatric disorders (e.g., depression, OCD, anxiety disorders, bipolar disorder) and extremes of personality traits have been described (Godart et al. (2000), Eur. Psychiatry 15: 38-45; Lilenfeld et al. (1998), Arch. Gen. Psychiatry 55: 603-610; Simpson et al. (1996), J. Nerv. Ment. Dis. 180: 719-722; Braun et al. (1994), Psychol. Med. 24: 859-867; Klump et al. (2000), J. Nerv. Ment. Dis. 188: 559-567). The relative risk is approximately 11 for AN and 4 for BN, (Strober et al. (1997), Int. J. Eat. Disorder 22: 339-360), and the additive genetic influence on the risk for eating disorders ranges between 50 and 80% (Kendler et al. (1991), Am. J. Psychiatry 148: 1627-1637; Wade et al. (1999), Psychol. Med. 29: 925-934; Bulik et al. (1998), Biol. Psychiatry 44: 1210-1218). Moreover, people with AN tend to have a unique cluster of personality and temperamental traits including perfectionism, over control, rigidity; and harm avoidance. Such behaviors may constitute predisposing traits since they occur premorbidly and frequently persist well after weight and eating normalize. These family history and heritability studies provide the required evidence to justify a molecular genetic approach to the study of eating disorder susceptibility factors.

AN, among the most disabling and lethal of psychiatric disorders, is often resistant to treatment, especially over the long term (Walsh and Devlin (1998), Science 280: 1387-1390). AN patients are at an increased risk for several traits such as obsessive-compulsive behavior, perfectionism, and anxious personality (Hinney et al. (2000), Eur. J. Pharmacol. 410:147-159; Kaye et al. (1999), Biol. Psychiatry 45: 1285-1292; Kaye et al. (2000), Annu. Rev. Med. 51: 299-313). In addition, an increased risk is present for the development of BN: about one-third of patients who present for treatment with BN have past histories of AN. In addition to the increased risk of AN among 1st degree relatives of AN probands, twin studies show higher concordance rates for monozygotic versus dizygotic twins, with heritability estimates ranging from 0.5-0.8 (Bulik et al. (2000)). AN is highly likely to be a complex disorder, influenced by multiple genes as well as environmental risk factors (Kaye et al. (2000)).

Candidate Gene Association Studies

Studies of AN have focused primarily on genes involved in body weight regulation (i.e., leptin gene, melanocortin receptor gene) and the serotonergic system. Most of these analyses genotype existing DNA polyniorphisnis in both affected individuals and unaffected individuals and then perform association analysis of allele and genotype frequencies to affection status (Diagnostic and Statistical Manual of Mental Disorders, 3rd Edition (DSM-IIIR) or DSM-IV AN and BN).

Six serotonergic candidate genes have been the subject of publications, i.e., the receptors 1B, 2A, 2C and 7, the serotonin transporter, and tryptophan hydroxylase, the rate limiting enzyme for serotonin synthesis in human brain. Association studies between a polymorphism at the serotonin receptors 1B and 7 and AN have been negative (Hinney et al. (1999) Int. J. Obes. Relat. Metab. Disord. 23: 760-763). A sequence polymorphism flanking the HTR2A locus (-1438G>A) has been associated with both AN and OCD, see e.g., Collier et al. (1997), Lancet 350: 412; Enoch et al. (1998), Lancet 351: 1785-1786; and Enoch et al. (2001), Biol. Psychiatry 49: 385-388. The summary literature based odds ratio for association with AN across seven studies with 665 cases and 1124 controls is 1.47, suggesting that the reported association is real. A functional HTR2C amino acid polymorphism associated with 3-methoxy-4-hydroxyphenylethyleneglycol (MPHG), the major metabolite of norepinephrine (Cys23Ser, Lappalainen et al. (1999), Biol. Psychiatry 46: 821-826; Lappalainen et al. (1995), Genomics 27: 274-279) has been previously associated with hyperphagia and auditory hallucinations in Alzheimer's disease (Holmes et al. (1998), Hum. Mol. Genet. 7: 1507-1509) and HTR1A knock-out mice have an obese phenotype (Tecott et al. (1995) Nature 374: 542-546) however, neither receptor is associated with anorexia or bulimia.

Typically, a single polymorphism at most of these genes has been studied in a case control design by one or two groups of investigators, although the serotonin 2A receptor gene (HTR2A) and the serotonin transporter gene (SLC6A4) have received attention from many groups. The mean eating disorder sample size (AN or BN) is about 85 individuals, while the mean control sample is about 170 individuals, however, control samples are very diverse and consist of both psychiatrically screened and unscreened samples, normal weight, underweight, and obese samples, and of both sexes. Only a few groups have samples which include parents or unaffected siblings (sibs). Family samples permit both association analysis and linkage and reduce the probability of type I error (false positives) at the cost of increased type II error (false negatives). Published family samples include parents from up to 55 families and 45 unaffected siblings, respectively, however, a recent study has described a family sample of approximately 300 trios that is the subject of molecular genetic investigation for eating and metabolic disorders (Hinney et al. (2000), Eur. J. Pharmacol. 410: 147-159). Any positive association findings in case control samples must be evaluated in additional case control samples and in family samples to evaluate the proposed relationship between sequence variation and risk for an eating disorder.

Description of the Serotonint Receptor 1D Gene and Protein

To date, no scientific accounts of associations between the serotonin receptor ID and eating disorders have been published. The serotonin receptor 1D, GenBank Record OMIM#182133 and GenBank Accession No. AL353585 (SEQ ID NO: 1), both of which are incorporated herein by reference, is a G protein-coupled receptor. The cloning deduced amino acid sequences, pharmacologic properties, and second-messenger coupling of a pair of human serotonin receptor 1D genes was described by Weinshank et al. (Proc. Nat.l Acad. Sci. U.S.A. 89: 3630-3634 (1992). They designated the genes 1Dα (HTR1D) and 1Dβ (HTR1B) due to their strong similarities.

The gene encoding HTR1D has been isolated, and it is reported to have no introns in its coding region and to consist of 377 amino acids (1134 bp) (Hamblin and Metcalf (1991), Mol. Pharmacol. 40: 143-148). It has been located to chromosome 1 at 1p36.3-p34.3 (Libert et al. (1991), Genomics 11: 225-227; Jin et al. (1992), J. Biol. Chem. 267: 5735-5738. The HTR1B gene has been assigned to chromosome 6 at q13 (Jin et al. (1992)). The amino acid sequence encoded by HTR1D exhibits approximately 55% identity with that of the HTR1B. The pharmacologic binding properties match closely those off human, bovine, and guinea pig serotonin receptor 1D sites. Both receptor genes are expressed in the human cerebral cortex, and the receptors are coupled to the inhibition of adenylate cyclase activity. Serotonin receptors maybe involved in blood circulation, locomotor activity, and body temperature regulation (Zifa and Fillion (1992), Pharmacol. Rev. 44: 401-458).

The Dopaminie System and Eating Disorders

AN has been classified as a primary eating disorder and/or a mood disorder that leads to decreased food intake. The dopaminergic system is involved in both cases. Dopamine release is known to be associated with enjoyable and satisfying events, and it is thought that it may reinforce positive aspects of feeding (Szczypka et al. (2000) Nat. Genet. 25: 102-104). It may work by helping to integrate the sensory cues related to hunger. In the Szczypka study, mice that were dopamine-deficient gradually became aphagic and died of starvation. The dopamine-deficient mice that were administered L-DOPA had restored locomotion and feeding. Dopamiine receptors have been implicated in numerous disorders, e.g. schizophrenia, Parkinson's disease, Tourette's syndromne, tardive dyskinesia, and Huntington's disease (Cravchik et al. (1996), J. Biol. Chem. 271: 26013-26017). Furthermore, recovered restricting anorexics have been shown to have significantly decreased dopamine metabolite (homovanillic acid (HVA)) levels, perhaps resulting from a trait-related disturbance in dopamine metabolism (Kaye et al. (1999), Biol. Psychiatry 45: 1285-1292).

Currently there are five known human dopamine receptors, D1, D2, D3, D4, and D5. According to their pharmacological properties and physical functions, the receptors can be divided into two subfamilies, D1-like (D1 and D5) and D2-like (D2, D3, and D4) (Cravchlik et al.). Of the D2-like receptors, the dopamine receptor D2 (DRD2) is the predominant receptor in the brain and is found at high levels in typical dopamine rich brain areas.

It has recently been shown that people suffering from obesity have fewer DRD2s than normal-weight subjects (Wang et al. (2001), Lancet 357: 354-357). Studies on the dopamine receptors D3 and D4 have not demonstrated an association between either one of the receptors and AN (Hinney et al. (1999), Am. J. Med. Genet. 88: 594-597; Bruins-Slot et al. (1998), Biol. Psychiatry 43: 76-78).

There are two additional studies of a dopaminergic gene, COMT (Karwautz et al. (2001), Psychol. Med. 31: 317-329; Frisch et al. (2001), Molecular Psychiatry 6: 243-245), where a study of twins (N=45) discordant for AN did not observe association to COMT alleles (Karwautz et al., 2001), and where a study of AN probands, parents and controls observed statistically significant excess transmission of the high activity allele of COMT in a trio sample and statistically significantly allelic association (with the high activity allele in excess) with AN in a case:control sample (both p=0.015) (Frisch et al., 2001). These phenotypic, neurochemical and genetic associations to eating behavior and anorexia support further investigation of dopaminergic loci, such as DRD2.

Description of the Dopamine Receptor D2 Gene and Protein

The dopamine receptor D2 (DRD2), GenBank Record OMIM No.126450 and GenBank Accession No. AF050737 (SEQ ID NO: 2), both of which are incorporated herein by reference, is a seven transmembrane G protein-linked receptor that binds dopamine and inhibits adenylate cyclase (Kebabian and Calne (1979), Nature 277: 93-96) and interacts with other transmembrane receptors and cellular proteins (Rocheville et al. (2000), Science 288: 154-157). The D2 receptor has been the subject of intensive study because of its role in dopaminergic mediated reward states (Wise and Bozarth (1984), Brain Res. Bull. 12: 203-208) and in the so-called reward deficiency syndrome (Comings and Blum (2000), Prog. Brain Res. 126: 325-341). A genetic polymorphic marker became available in 1989 for association studies (Grandy et al. (1989), Am. J. Hum. Genet. 45: 778-785). Agonists of DRD2, such as apomorphine, have been shown to be anorexigenic (Barzaghi et al. (1973), J. Pharm. Pharmacol. 25: 909-911).

The DRD2 gene extends over 270 kb and includes an intron of approximately 250 kb separating the first exon from the exons that encode the receptor protein (Eubanks et al. (1992), Genomics 14: 1010-1018). Awareness of the inadequacy of association studies using single polymorphisms and convenience control samples (Gelernter et al. (1993), JAMA 269: 1673-1677) suggests that candidate gene analysis must take into account the available genomic data, putatively functional polymorphisms, and population genetic information.

Description of the Delta-opioid Receptor Gene and Protein

Similar to the dopaminergic system, the opioid system is involved in controlling pain, reward, and addiction. The delta-opioid receptor (OPRD1) gene, Genbank Record OMIM No. 165195 and GenBank Accession No. U07882 (SEQ ID NO: 3), both of which are incorporated herein by reference, contains three exons encoding a seven-transmembrane, G protein-coupled receptor (Zaki et al. (1996), Annu. Rev. Pharmacol. Toxicol. 36: 379-401). No studies have linked the OPRD1 gene to a role in AN or BN.

### SUMMARY OF THE INVENTION

The present invention is based on the discovery of nucleotide polymorphisms in genes whose products are involved in serotonin, dopamine, noradrenergic and opioidergic neurotransmission and in the central nervous system control of appetite regulation. More specifically, the present invention is based on the discovery of nucleotide polymorphisms in the HTR1D, OPRD1, and the DRD2 genes and the association and linkage of these polymorphisms with an eating disorder such as AN or BN.

In the present specification, the differences in allele, haplotype, and genotype frequencies of seven SNPs at the DRD2 gene locus, four SNPs at the HTR1D locus, and five SNPs at the OPRD1 locus are evaluated in a sample of individuals fulfilling DSM-IV AN criteria, ARPs with a DMS-IV eating disorder diagnosis, and related family members versus unrelated, female, normal weight, DSM-IIIR Axis I screened negative controls.

In some aspects of the present invention, the differences in allele, haplotype, and genotype frequencies of one or more of the SNPs listed in Table 1 may be evaluated in a sample derived from a subject to be tested. The subject may have symptoms of an eating disorder or may be asymptomatic.

In another apsect of the present invention, kits suitable for the diagnosis of a predisposition to an eating disorder are provided. The kits may comprise one or more oligonucleotides suitable for identifying a nucleotide present at a SNP position. In some preferred embodiments, one or more of the oligonucleotides may have a sequence such that the 3′-terminal nucleotide of the oligonucleotide is aligned with the SNP position.

The present invention also provides databases comprising information related to the polymorphisms of the present invention. In some aspects, the present invention provides a database comprising SNP allele frequency information on one or more SNPs identified as associated with eating disorders, wherein the database is on a computer-readable medium. The databases of the invention preferably comprise information on at least one of the SNPs identified in Table 1. The databases of the present invention may optionally comprise information on one or more factors selected from a group consisting of environmental factors, other genetic factors, related factors, including but not limited to biochemical markers, behaviors, and/or other polymorphisms, including but not limited to low frequency SNPs, repeats, insertions and deletions.

### SPECIFIC EMBODIMENTS

Current treatments for AN or BN are aimed at normalizing body weight, correcting the irrational preoccupation with weight loss, and preventing weight loss. Although many patients eventually make full recoveries, the long-term outcome is disappointing in at least 50% of cases. The frequency of depression is high, and social and occupational functioning is often impaired, while many individuals never achieve a normal body weight (Walsh and Devlin (1998), Science 280: 1387-1390). The mortality, due to complications of starvation or from suicide is substantial, approximately 5% per decade of follow-up (Sullivan (1995), Am. J. Psychiatry 152: 1073-1074). No pharmacological agent has been established to be of benefit in the treatment of AN (Mayer and Walsh (1998), J. Clin. Psychiatry 59: 28-34). With a better understanding of the biological mechanisms involved in AN and BN, medication may be developed, that could improve the success of future treatment programs.

Previous studies have indicated the possible involvement of serotonin, opioids, and dopamine in eating disorders. From a biological standpoint, genes involved in serotonin, opioid, and dopamine regulation appear to be good candidate genes for eating disorders, because they have all been associated with two aspects that are important in eating disorders, food intake as well as mood.

The present study has discovered the involvement of the HTR1D, OPRD1, and DRD2 gene loci in AN. Seven SNPs at the DRD2 gene locus, four SNPs at the HTR1D locus, and five SNPs at the OPRD1 locus were typed in a sample of anorectic probands as well as in two control samples. Statistically significant genotypic, allelic, and haplotypic association to AN in the case: control design was observed at HTR1D and OPRD1 with effect sizes for individual SNPs of 2.63 (95% CI=1.21-5.75) for HTR1D and 1.61 (95% CI=1.11-2.44) for OPRD1. Using genotype data on parents and AN probands, three SNPs at HTR1D were found to exhibit significant transmission disequilibrium (p<0.05).

Allele and genotype absolute and relative frequencies in the AN, AN1 and AN2 proband samples and in the EAF control samples are shown in Table 2. DRD2-23 (SNP000000181), which was genotyped in the family dataset only, was uncommon with a minor allele frequency of 2% in the AN probands, and was present in only 11 of the affected relatives and parents and was not included in the genotypic and allelic association and transmission disequilibrium analyses. The observed completion rate in the AN proband sample for the DRD2 SNPs DRD2-43 (p000062594), DRD2-11 (SNP000003288), DRD2-23 (SNP000000181), DRD2-24 (SNP000000403), DRD2-25 (SNP000006629), DRD2-35 (SNP000007297), and DRD2-42 (SNP000003286), was 97%, 86%, 95%, 96%, 96%, 86%, and 92% (mean=93+/−0.05%). The observed completion rate in the EAF sample for the DRD2 SNPs DRD2-43, DRD2-11, DRD2-24, DRD2-25, and DRD2-42 was 96%, 87%, 94%, 87%, 90%, 86% (mean=90+/−0.04%). The observed discordance rate for DRD2-11, DRD2-24, DRD2-25, DRD2-35, and DRD2-42 based on duplicated samples was 0% and for DRD2-43 was 5.9%. Upon review, the observed discordances at DRD2-43 were consistent with either incomplete digestion or, in one case, lack of digestion, in the BstNI RFLP genotyping assay. The number of observed non-Mendelian transmissions at DRD2-43 and DRD2-23 was zero, at DRD2-24 was one, and at DRD2-25 was two. All genotypes identified as discordant or exhibiting non-Mendelian transmission were dropped from further analysis. DRD2 SNPs genotyped in the AN, AN1, AN2 proband and EAF samples were in HWE equilibrium (p>0.05). There were 25 tests of HWE conducted, 17 of which were independent tests (AN1 and AN2 proband samples derived from the AN proband sample were not independent tests).

The DRD2-43 SNP was found to be statistically significantly associated with DSM-IV AN (genotypic, allelic and haplotypic) in case:control contingency analysis and to exhibit transmission disequilibrium (allelic). In the present invention, the DRD2-43 deletion allele was less frequent in AN probands (5.9%) than in the EAF control sample (11.2%). The DRD2-43 deletion allele frequency in the EAF control sample in the present invention (0.1124+/−0.0237 (S.E.)) was consistent with (Pearson χ²=0.198, p=0.6564) the estimated crude DRD2-43 deletion allele frequency in ten different Caucasian control samples in the literature (unweighted crude average=0.0996+/−0.0169 (S.D.), crude average=0.10164+/−0.00558 (S.E.), total N of combined control sample is 1466) (Breen et al. 1999, Am. J. Med. Genet. 88: 407-410; Gelernter et al. 1998, Genomics 51: 21-26; Gelernter et al. 1999, Neuropsychopharmacology 20: 640-649; Furlong et al. 1998, Am. J. Med. Genet. 81: 385-387; Jonsson et al. 1999, Schizophr. Res. 40: 31-36; Li et al. 1998, Schizophr. Res. 32: 87-92; Noble et al. 2000, Am. J. Med. Genet. 96: 622-631, Parsian et al. 2000, Am. J. Med. Genet. 96: 407-411; Tallerico et al. 1999, Psychiatry Res. 85: 215-219). The estimated crude average of the DRD2-43 deletion allele frequency in thirteen samples composed of Caucasian individuals affected with schizophrenia, bipolar disorder or alcoholism schizophrenia, bipolar disorder or alcoholism (unweighted crude average=0.10863+/−0.02928 (S.D.), crude average=0.11700+/−0.00541 (S.E.), total N of combined case samples is 1636) (Arranz et al. 1998, Pharmacogenetics 8: 481-484; Breen et al. 1999; Blomqvist et al. 2000, Am. J. Med. Genet. 96: 659-664; Gelernter et al. 1999; Furlong 1998; Jonsson et al. 1999; Li et al. 1998; Noble 2000; Parsian 2000; Tallerico et al. 1999) was significantly greater than the crude control DRD2-43 deletion allele frequency average (Pearson χ²=3.859, p=0.0494) and was similar to the EAF sample DRD2-43 deletion allele frequency (Pearson χ²=0.035, p=0.851). This assessment of crude DRD2-43 deletion allele frequency estimates is in contrast to the current invention, wherein a statistically significantly lower DRD2-43 deletion allele frequency was observed in the AN sample compared to the EAF sample.

A. Definitions

As used herein, the terms “serotonin receptor 1B,” “serotonin receptor 1B gene” or “HTR1B” refer to any mammalian serotonin receptor 1B gene or protein, and in particular, although not limited to, human serotonin receptor 1B genes and proteins. As described above, the human HTR1B gene has been cloned, expression has been mapped, and the gene localized to chromosome 6 in the human. The terms “serotonin receptor 1B,” “serotonin receptor 1B gene” or “HTR1B,” however, are not limited to these specific sequences. For instance, the terms also refer to naturally occurring subtypes and allelic variants, as well as to man-made substitution, insertion or deletion mutants that have a slightly different amino acid sequence than those specifically referred to above.

As used herein, the family of proteins related to the human amino acid sequence of HTR1B refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “serotonin receptor 1B variant,” “serotonin receptor 1B polymorphism,” “HTR1B valiant” or “HTR1B polymorphism,” as well as the gene encoding either the HTR1B variant or polymorphism refers to a form of the receptor or its encoding gene that is associated with a genetic predisposition to an eating disorder, such as AN or BN.

As used herein, the term “serotonin-1B-receptor-mediated disease” or “HTR1B-mediated disease” refers to a disorder or pathology in which the presence of an “HTR1B variant” or “HTR1B polymorphism” is associated with or participates in a signaling or other biological pathway in a manner that results in a pathological condition such as those eating and energy metabolism disorders identified above.

As used herein, the terms “serotonin receptor 1D,” “serotonin receptor 1D gene” or “HTR1D” refer to any mammalian serotonin receptor 1D gene or protein, and in particular, although not limited to, human serotonin receptor 1D genes and proteins. As described above, the human HTR1D gene has been cloned, expression has been mapped, and the gene localized to chromosome 1 in the human. The terms “serotonin receptor 1D,” “serotonin receptor 1D gene” or “HTR1D,” however, are not limited to these specific sequences. For instance, the terms also refer to naturally occurring subtypes and allelic variants, as well as to man-made substitution, insertion or deletion mutants that have a slightly different amino acid sequence than those specifically referred to above.

As used herein, the family of proteins related to the human amino acid sequence of HTR1D refers to proteins that have been isolated from organisms in addition to humans. The methods used to identify and isolate other members of the family of proteins related to these proteins are readily available and known to persons skilled in the molecular biology field, including hybridization and sequence or homology screeniing methods.

As used herein, the terms “serotonin receptor 1D variant,” “serotonin receptor 1D polymorphism,”,“HTR1D variant” or “HTR1D polymorphism,” as well as the gene encoding either the HTR1D variant or polymorphism refers to a form of the receptor or its encoding gene that is associated with a genetic predisposition to an eating disorder, such as AN or BN.

As used herein, the term “serotonin-1D-receptor-mediated disease” or “HTR1D-mediated disease” refers to a disorder or pathology in which the presence of an “HTR1D variant” or “HTR1D polymorphism” is associated with or participates in a signaling or other biological pathway in a manner that results in a pathological condition such as those eating and energy metabolism disorders identified above.

As used herein, the terms “serotonin receptor 2A,” “serotonin receptor 2A gene” or “HTR2A” refer to any mammalian serotonin receptor 2A gene or protein, and in particular, although not limited to, human serotonin receptor 2A genes and proteins. As described above, the human HTR2A gene has been cloned, expression has been mapped, and the gene localized to chromosome 13 in the human. The terms “serotonin receptor 2A,” “serotonin receptor 2A gene” or “HTR2A,” however, are not limited to these specific sequences. For instance, the terms also refer to naturally occurring subtypes and allelic variants, as well as to man-made substitution, insertion or deletion mutants that have a slightly different amino acid sequence than those specifically referred to above.

As used herein, the family of proteins related to the human amino acid sequence of HTR2A refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “serotonin receptor 2A variant,” “serotonin receptor 2A polymorphism,”,“HTR2A variant” or “HTR2A polymorphism,” as well as the gene encoding either the HTR2A variant or polymorphism refers to a form of the receptor or its encoding gene that is associated with a genetic predisposition to an eating disorder, such as AN or BN.

As used herein, the term “serotonin-2A-receptor-mediated disease” or “HTR2A-mediated disease” refers to a disorder or pathology in which the presence of an “HTR2A variant” or “HTR2A polymorphism” is associated with or participates in a signaling or other biological pathway in a manner that results in a pathological condition such as those eating and energy metabolism disorders identified above.

As used herein, the terms “serotonin receptor 2C,” “serotonin receptor 2C gene” or “HTR2C” refer to any mammalian serotonin receptor 2C gene or protein, and in particular, although not limited to, human serotonin receptor 2C genes and proteins. As described above, the human HTR2C gene has been cloned, expression has been mapped, and the gene localized to the X chromosome in the human. The terms “serotonin receptor 2C,” “serotonin receptor 2C gene” or “HTR2C,” however, are not limited to these specific sequences. For instance, the terms also refer to naturally occurring subtypes and allelic variants, as well as to man-made substitution, insertion or deletion mutants that have a slightly different amino acid sequence than those specifically referred to above.

As used herein, the family of proteins related to the human amino acid sequence of HTR2C refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “serotonin receptor 2C variant,”. “serotonin receptor 2C polymorphism,” “HTR2C variant” or “HTR2C polymorphism,” as well as the gene encoding either the HTR2C variant or polymorphism refers to a form of the receptor or its encoding gene that is associated with a genetic predisposition to an eating disorder, such as AN or BN.

As used herein, the term “serotonin-2C-receptor-mediated disease” or “HTR2C-mediated disease” refers to a disorder or pathology in which the presence of an “HTR2C variant” or “HTR2C polymorphism” is associated with or participates in a signaling or other biological pathway in a manner that results in a pathological condition such as those eating and energy metabolism disorders identified above.

As used herein, the terms “serotonin receptor 5A,” “serotonin receptor 5A gene” or “HTR5A” refer to any mammalian serotonin receptor 5A gene or protein, and in particular, although not limited to, human serotonin receptor 5A genes and proteins. As described above, the human HTR5A gene has been cloned, expression has been mapped, and the gene localized to chromosome 7 in the human. The terms “serotonin receptor 5A,” “serotonin receptor 5A gene” or “HTR5A,” however, are not limited to these specific sequences. For instance, the terms also refer to naturally occurring subtypes and allelic variants, as well as to man-made substitution, insertion or deletion mutants that have a slightly different amino acid sequence than those specifically referred to above.

As used herein, the family of proteins related to the human amino acid sequence of HTR5A refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “serotonin receptor 5A variant,” “serotonin receptor 5A polymorphism,” “HTR5A variant” or “HTR5A polymorphism,” as well as the gene encoding either the HTR5A variant or polymorphism refers to a form of the receptor or its encoding gene that is associated with a genetic predisposition to an eating disorder, such as AN or BN.

As used herein, the term “serotonin-5A-receptor-mediated disease” or “HTR5A-mediated disease” refers to a disorder or pathology in which the presence of an “HTR5A variant” or “HTR5A polymorphism” is associated with or participates in a signaling or other biological pathway in a manner that results in a pathological condition such as those eating and energy metabolism disorders identified above.

As used herein, the terms “delta-opioid receptor”, “delta-opioid receptor gene” or “OPRD1” refer to any mammalian s delta-opioid receptor gene or protein, and in particular, although not limited to, human delta-opioid receptor genes and proteins. As described above, the human OPRD1 gene has been cloned, expression has been mapped, and the gene localized to chromosome 1 in the human. The terms “delta-opioid receptor,” “delta-opioid receptor gene” or “OPRD1, ” however, are not limited to these specific sequences. For instance, the terms also refer to naturally occurring subtypes and allelic variants, as well as to man-made substitution, insertion or deletion mutants that have a slightly different amino acid sequence than those specifically referred to above.

As used herein, the family of proteins related to the human amino acid sequence of OPRD1 refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “delta-opioid receptor variant,” “delta-opioid receptor polymorphism,” “OPRD1 variant” or “OPRD1 polymorphism,” as well as the gene encoding either the OPRD1 variant or polymorphism refers to a form of the receptor or its encoding gene that is associated with a genetic predisposition to an eating disorder, such as AN or BN.

As used herein, the terms “dopamine receptor D1,” “dopamine receptor D1 gene” or “DRD1” refer to any mammalian dopamine receptor D1 gene or protein, and in particular, although not limited to, human dopamine receptor D1 genes and proteins. As described above, the human DRD1 gene has been cloned, expression has been mapped, and the gene localized to chromosome 5 in the human. The terms “dopamine receptor D1,” “dopamine receptor D1 gene” or “DRD1,” however, are not limited to specific sequences. For instance, the terms also refer to naturally occurring subtypes and allelic variants, as well as to man-made substitution, such as insertion or deletion mutants that have a slightly different amino acid sequence than those specifically referred to, above.

As used herein, the family of proteins related to the human amino acid sequence of DRD1 refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “dopamine receptor D1 variant,” “dopamine receptor D1 polymorphism,” “DRD1 variant” or “DRD 1 polymorphism,” as well as the gene encoding either the DRD1 variant or polymorphism refers to a form of the receptor or its encoding gene that is associated with a genetic predisposition to an eating disorder, such as AN or BN.

As used herein, the term “dopamine-D1-receptor-mediated disease” or “DRD1-mediated disease” refers to a disorder or pathology in which the presence of a “DRD1 variant” or “DRD1 polymorphism” is associated with or participates in a signaling or other biological pathway in a manner that results in a pathological condition such as those eating and energy metabolism disorders identified above.

As used herein, the term “delta-opioid receptor-mediated disease” or “OPRD1-mediated disease” refers to a disorder or pathology in which the presence of an “OPRD1 variant” or “OPRD1 polymorphism” is associated with or participates in a signaling or other biological pathway in a manner that results in a pathological condition such as those eating and energy metabolism disorders identified above.

As used herein, the terms “dopamine receptor D2, ” “dopamine receptor D2 gene” or “DRD2” refer to any mammalian dopamine receptor D2 gene or protein, and in particular, although not limited to, human dopamine receptor D2 genes and proteins. As described above, the human DRD2 gene has been cloned, expression has been mapped, and the gene localized to chromosome 11 in the human. The terms “dopamine receptor D2,” “dopamine receptor D2 gene” or “D1RD2,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of DRD2 refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “dopamine receptor D2 variant,” “dopamine receptor D2 polymorphism,” “DRD2 variant” or “DRD2 polymorphism,” as well as the gene encoding either the DRD2 variant or polymorphism refers to a form of the receptor or its encoding gene that is associated with a genetic predisposition to an eating disorder, such as AN or BN.

As used herein, the term “dopamine-D2-receptor-mediated disease” or “DRD2-mediated disease” refers to a disorder or pathology in which the presence of a “DRD2 variant” or “DRD2 polymorphism” is associated with or participates in a signaling or other biological pathway in a manner that results in a pathological condition such as those eating and energy metabolism disorders identified above.

As used herein, the terms “dopamine receptor D3,” “dopamine receptor D3 gene” or “DRD3” refer to any mammalian dopamine receptor D3 gene or protein, and in particular, although not limited to, human dopamine receptor D3 genes and proteins. As described above, the human DRD3 gene has been cloned, expression has been mapped, and the gene localized to chromosome 3 in the human. The terms “dopamine receptor D3,” “dopamine receptor D3 gene” or “DRD3,”however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of DRD3 refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “dopamine receptor D3 variant,” “dopamine receptor D3 polymorphism,” “DRD3 variant” or “DRD3 polymorphism,” as well as the gene encoding either the DRD3 variant or polymorphism refers to a form of the receptor or its encoding gene that is associated with a genetic predisposition to an eating disorder, such as AN or BN.

As used herein, the term “dopamine-D3-receptor-mediated disease” or “DRD3-mediated disease” refers to a disorder or pathology in which the presence of a “DRD3 variant” or “DRD3 polymorphism” is associated with or participates in a signaling or other biological pathway in a manner that results in a pathological condition such as those eating and energy metabolism disorders identified above.

As used herein, the terms “dopamine receptor D4,” “dopamine receptor D4 gene” or “DRD4” refer to any mammalian dopamine receptor D4 gene or protein, and in particular, although not limited to, human dopamine receptor D4 genes and proteins. As described above, the human DRD4 gene has been cloned, expression has been mapped, and the gene localized to chromosome 11 in the human. The terms “dopamine receptor D4,” “dopamine, receptor D4 gene” or “DRD4,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of DRD4 refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “dopamine receptor D4 variant,” “dopamine receptor D4 polymorphism,” “DRD4 variant” or “DRD4 polymorphism,” as well as the gene encoding either the DRD4 valiant or polymorphism refers to a form of the receptor or its encoding gene that is associated with a genetic predisposition to an eating disorder, such as AN or BN.

As used herein, the term “dopamine-D4-receptor-mediated disease” or “DRD4-mediated disease” refers to a disorder or pathology in which the presence of a “DRD4 variant” or “DRD4 polymorphism” is associated with or participates in a signaling or other biological pathway in a manner that results in a pathological condition such as those eating and energy metabolism disorders identified above.

As used herein, the terms “hypocretin receptor 2,” “hypocretin receptor 2 gene,” “orexin 2 receptor,” “orexin 2 receptor gene” or “HCRTR2” refer to any mammalian hypocretin receptor 2 gene or protein, and in particular, although not limited to, human hypocretin receptor 2 genes and proteins. As described above, the human HCRT2 gene has been cloned, expression has been mapped, and the gene localized to chromosome 6 in the human. The terms “hypocretin receptor 2,” “hypocretin receptor 2 gene,” “orexin 2 receptor,” “orexin 2 receptor gene” or “HCRTR2,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of HCRTR2 refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “hypocretin receptor 2 variant,” “hypocretin receptor 2 polymorphism,” “orexin 2 receptor variant,” “orexin 2 receptor polymorphism,” “HCRTR2 variant” or “HCRTR2 polymorphism” as well as the gene encoding either the HCRTR2 variant or polymorphism refers to a form of the receptor or its encoding gene that is associated with a genetic predisposition to an eating disorder, such as AN or BN.

As used herein, the term “hypocretin receptor 2-mediated disease” or “HCRTR2-mediated disease” refers to a disorder or pathology in which the presence of a “HCRTR2 variant” or “HCRTR2 polymorphism” is associated with or participates in a signaling or other biological pathway in a manner that results in a pathological condition such as those eating and energy metabolism disorders identified above.

As used herein, the terms “dopamine beta-hydroxylase,” “dopamine beta-hydroxylase gene” or “DBH” refer to any mammalian dopamine beta-hydroxylase gene or protein, and in particular, although not limited to, human dopamine beta-hydroxylase genes and proteins. As described above, the human DBH gene has been cloned, expression has been mapped, and the gene localized to chromosome 9 in the human. The terms “dopamine beta-hydroxylase,” “dopamine beta-hydroxylase gene” or “DBH,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of DBH refers to, proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “dopamine beta-hydroxylase variant,” “dopamine beta-hydroxylase polymorphism,” “DBH variant” or “DBH polymorphism,” as well as the gene encoding either the DBH variant or polymorphism refers to a form of the protein or its encoding gene that is associated with a genetic predisposition to an eating disorder, such as AN or BN.

As used herein, the term “dopamine beta-hydroxylase-mediated disease” or “DBH-mediated disease” refers to a disorder or pathology in which the presence of a “DBH variant” or “DBH polymorphism” is associated with or participates in a signaling or other biological pathway in a manner that results in a pathological condition such as those eating and energy metabolism disorders identified above.

As used herein, the terms “tyrosine hydroxylase,” “tyrosine hydroxylase gene” or “TH” refer to any mammalian tyrosine hydroxylase gene or protein, and in particular, although not limited to, human tyrosine hydroxylase genes and proteins. As described above, the human TH gene has been cloned, expression has been mapped, and the gene localized to chromosome 11 in the human. The terms “tyrosine hydroxylase,” “tyrosine hydroxylase gene” or “TH,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of TH refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “tyrosine hydroxylase variant,” “tyrosine hydroxylase polymorphism,” “TH variant” or “TH polymorphism,” as well as the gene encoding either the TH variant or polymorphism refers to a form of the protein or its encoding gene that is associated with a genetic predisposition to an eating disorder, such as AN or BN.

As used herein, the term “tyrosine hydroxylase-mediated disease” or “TH-mediated disease” refers to a disorder or pathology in which the presence of a “TH variant” or “TH polymorphism” is associated with or participates in a signaling or other biological pathway in a manner that results in a pathological condition such as those eating and energy metabolism disorders identified above.

As used herein, the terms “thyrotropin-releasing hormone,” “thyrotropin-releasing hormone gene” or “TRH” refer to any mammalian thyrotropin-releasing hormone gene or protein, and in particular, although not limited to, human thyrotropin-releasing hormone genes and proteins. As described above, the human TRH gene has been cloned, expression has been mapped, and the gene localized to chromosome 3 in the human. The terms “thyrotropin-releasing hormone,” “thyrotropin-releasing hormone gene” or “TRH,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of TRH refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “thyrotropin-releasing hormone variant,” “thyrotropin-releasing hormone polymorphism,” “TRH variant” or “TRH polymorphism,” as well as the gene encoding either the TRH variant or polymorphism refers to a form of the protein or its encoding gene that is associated with a genetic predisposition to an eating disorder, such as AN or BN.

As used herein, the term “thyrotropin-releasing hormone-mediated disease” or “TRH-mediated disease” refers to a disorder or pathology in which the presence of a “TRH variant” or “TRH polymorphism” is associated with or participates in a signaling or other biological pathway in a manner that results in a pathological condition such as those eating and energy metabolism disorders identified above.

As used herein, the terms “thyrotropin-releasing hormone receptor,” “thyrotropin-releasing hormone receptor gene” or “TRHR” refer to any mammalian thyrotropin-releasing hormone receptor gene or protein, and in particular, although not limited to, human thyrotropin-releasing hormone receptor genes and proteins. As described above, the human TRHR gene has been cloned, expression has been mapped, and the gene localized to chromosome 8 in the human. The terms “thyrotropin-releasing hormone receptor,” “thyrotropin releasing hormone receptor gene” or “TRHR,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of TRHR refers to proteins that have been isolated from organisms in addition to humans. The methods used to identify and isolate other members of the family of proteins related to these proteins are readily available and kwown to persons skilled in the molecular biology field, including hybridization and sequence or homology screening methods.

As used herein, the terms “thyrotropin-releasing hormone receptor variant,” “thyrotropin-releasing hormone receptor polymorphism,” “TRHR variant” or “TRHR polymorphism,” as well as the gene encoding either the TRHR variant or polymorphism refers to a form of the receptor or its encoding gene that is associated with a genetic, predisposition to an eating disorder, such as AN or BN.

As used herein, the term “thyrotropin-releasing hormone receptor-mediated disease” or “TRHR-mediated disease” refers to a disorder or pathology in which the presence of a “TRHR variant” or “TRHR polymorphism” is associated with or participates in a signaling or other biological pathway in a manner that results in a pathological condition such as those eating and energy metabolism disorders identified above.

As used herein, the terms “serotonin transporter,” “serotonin transporter gene” or “5HTT” refer to any mammalian serotonin transporter gene or protein, and in particular, although not limited to, human serotonin transporter genes and proteins. As described above, the human 5HTT gene has been cloned, expression has been mapped, and the gene localized to chromosome 17 in the human. The terms “serotonin transporter,” “serotonin transporter gene” or “5HTT,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of 5HTT refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “serotonin transporter variant,” “serotonin transporter polymorphism,” “5HTT variant” or “5HTT polymorphism,” as well as the gene encoding either the 5HTT variant or polymorphism refers to a form of the protein or its encoding gene that is associated with a genetic predisposition to an eating disorder, such as AN or BN.

As used herein, the term “serotonin transporter-mediated disease” or “5HTT-mediated disease” refers to a disorder or pathology in which the presence of a “5HTT variant” or “5HTT polymorphism” is associated with or participates in a signaling or other biological pathway in a manner that results in a pathological condition such as those eating and energy metabolism disorders identified above.

As used herein, the terms “G protein alpha subunit,” “G protein alpha subunit gene,” “G-alpha-OLF” or “GOLF” refer to any mammalian G protein alpha subunit gene or protein involved in olfaction, and in particular, although not limited to, human G protein alpha subunit genes and proteins involved in olfaction. As described above, the human GOLF gene has been cloned, expression has been mapped, and the gene localized to chromosome 18 in the human. The terms “G protein alpha subunit,” “G protein alpha subunit gene,” “G-alpha-OLF” or “GOLF,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of GOLF refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “G protein alpha subunit variant,” “G protein alpha subunit,” “GOLF variant” or “GOLF polymorphism,” as well as the gene encoding either the GOLF variant or polymorphism refers to a form of the protein or its encoding gene that is associated with a genetic predisposition to an eating disorder, such as AN or BN.

As used herein, the term “G protein alpha subunit variant-mediated disease” or “GOLF-mediated disease” refers to a disorder or pathology in which the presence of a “GOLF variant” or “GOLF polymorphism” is associated with or participates in a signaling or other biological pathway in a manner that results in a pathological condition such as those eating and energy metabolism disorders identified above.

As used herein, the terms “β1-adrenergic receptor,” “beta1-adrenergic receptor,” “β1-adrenergic receptor gene” or “ADRB1” refer to any mammalian adrenergic receptor β1gene or protein, and in particular, although not limited to, human adrenergic receptor β1 genes and proteins. As described above, the human ADRB1 gene has been cloned, expression has been mapped, and the gene localized to chromosome 10 in the human. The terms “β1-adrenergic receptor,” “beta1-adrenergic receptor,” “β1-adrenergic receptor gene” or “ADRB1,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of ADRB1 refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “β1-adrenergic receptor variant,” “beta1-adrenergic receptor variant,” “β1-adrenergic receptor polymorphism,” “ADRB1 variant” or “ADRB1 polymorphism,” as well as the gene encoding either the ADRB1 variant or polymorphism refers to a form of the receptor or its encoding gene that is associated with a genetic predisposition to an eating disorder, such as AN or BN.

As used herein, the term “β1-adrenergic receptor-mediated disease” or “ADRB1-mediated disease” refers to a disorder or pathology in which the presence of a “ADRB1 variant” or “ADRB1 polymorphism” is associated with or participates in a signaling or other biological pathway in a manner that results in a pathological condition such as those eating and energy metabolism disorders identified above.

As used herein, the terms “β2-adrenergic receptor,” “beta2-adrenergic receptor,” “β2-adrenergic receptor gene” or “ADRB2” refer to any mammalian adrenergic receptor β2 gene or protein, and in particular, although not limited to, human adrenergic receptor β2 genes and proteins. As described above, the human ADRB2 gene has been cloned, expression has been mapped, and the gene localized to chromosome 5 in the human. The terms “β2-adrenergic receptor,” “beta2-adrenergic receptor,” “β2-adrenergic receptor gene” or “ADRD2,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of ADRB2 refers to proteins that have been isolated from organisms in addition to humans. The methods used to identify and isolate other members of the family of proteins related to these proteins are readily available and known to persons skilled in the molecular biology field, including hybridization and sequence or homology screening methods.

As used herein, the terms “β2-adrenergic receptor variant,” “beta2-adrenergic receptor variant,” “β2-adrenergic receptor polymorphism,” “ADRB2 variant” or “ADRB2 polymorphism,” as well as the gene encoding either the ADRB2 variant or polymorphism refers to a form of the receptor or its encoding gene that is associated with a genetic predisposition to an eating disorder, such as AN or BN.

As used herein, the term “β2-adrenergic receptor-mediated disease” or “ADRB2-mediated disease” refers to a disorder or pathology in which the presence of a “ADRB2 variant” or “ADRB2 polymorphism” is associated with or participates in a signaling or other biological pathway in a manner that results in a pathological condition such as those eating and energy metabolism disorders identified above.

As used herein, the terms “catechol-O-methyltransferase,” “catechol-O-methyltransferase gene” or “COMT” refer to any mammalian catechol-O-methyltransferase gene or protein, and in particular, although not limited to, human catechol-O-methyltransferase genes and proteins. As described above, the human COMT gene has been cloned, expression has been mapped, and the gene localized to chromosome 22 in the human. The terms “catechol-O-methyltransferase,” “catechol-O-methyltransferase gene” or “COMT,” however, are not limited to specific sequences. For instance, the terms also refer to naturally occurring subtypes and allelic variants, as well as to man-made substitution, such as insertion or deletion mutants that have a slightly different amino acid sequence than those specifically referred to above.

As used herein, the family of proteins related to the human amino acid sequence of COMT refers to proteins that have been isolated from organisms in addition to humans. The methods used to identify and isolate other members of the family of proteins related to these proteins are readily available and known to persons skilled in the molecular biology field, including hyblidization and sequence or homology screening methods.

As used herein, the terms “catechol-O-methyltranisferase variant,” “catechol-O-methyltransferase polymorphism,” “COMT variant” or “COMT polymorphism,” as well as the gene encoding either the COMT variant or polymorphism refers to a form of the protein or its encoding gene that is associated with a genetic predisposition to an eating disorder, such as AN or BN.

As used herein, the term “catechol-O-methyltransferase-mediated disease” or “COMT-mediated disease” refers to a disorder or pathology in which the presence of a “COMT variant” or “COMT polymorphism” is associated with or participates in a signaling or other biological pathway in a manner that results in a pathological condition such as those eating and energy metabolism disorders identified above.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain such an isolated protein. Receptor proteins, or peptide fragments thereof may also be covalently modified by substitution, chemical, enzymatic, or other appropriate means with a moiety other than a naturally occurring amino acid (for example a detectable moiety such as an enzyme or radioisotope).

As used herein, a nucleic acid molecule is said to be “isolated” when the nucleic acid molecule is substantially separated from and relative to contaminant or other nucleic acid molecules encoding other polypeptides with which the nucleic acids of the present invention are customarily associated. Nucleic acid molecules of the invention may be cloned into any available vector for replication and/or expression in suitable host cells. The host cells then may be used to recombinantly produce the encoded protein. Appropriate vectors, host cells and methods of expression are widely available.

B. Methods of Using the Polymorphisms

The invention provides a method for the diagnosis of an HTR1D-, OPRD1-, DRD2, or other gene-mediated disease as herein described, such as an eating disorder, comprising the steps of detecting the presence or absence of a variant nucleotide at one or more of positions herein described in a patient sample and determining the status of the individual by reference to polymorphism in the HTR1D, OPRD1, or DRD2 gene. In preferred methods, a polymorphism is detected at a position corresponding to HTR1D-05, HTR1D-03, HTR1D-07, HTR1D-06, OPRD1-06, OPRD1-01, OPRD1-03, OPRD1-07 or OPRD1-05 as shown in Table 3, or at a position corresponding to DRD2-11, DRD2-23, DRD2-24, DRD2-25, DRD2-35, DRD2-42, and DRD2-43 as shown in Table 4.

Any sample comprising cells or nucleic acids from the patient or subject to be tested may be used. Preferred samples are those easily obtained from the patient or subject. Such samples include, but are not limited to blood, peripheral lymphocytes, epithelial cell swabs, bronchoalveolar lavage fluid, sputum, or other body fluid or tissue obtained from an individual. It will be appreciated that the test sample may comprise an HTR1D, OPRD1, DRD2, or other nucleic acid that has been amplified using any convenient technique, e.g., PCR, before analysis of allelic variation. As described below, any available means of detecting a sequence polymorphism(s) of the invention may be used in the methods.

In another method of the invention, the diagnostic methods described herein are used in the development of new drug therapies which selectively target one or more allelic variants of an HTR1D, OPRD1, DRD2, or other gene as herein described that are associated with an eating disorder. In one format, the diagnostic assays of the invention may be used to stratify patient populations by separating out patients with a genetic predisposition to an eating disorder from the general population. Identification of a link between a particular allelic variant and predisposition to disease development or response to drug therapy may have a significant impact on the design of new drugs by assisting in the analysis of a drugs efficacy or effects on specific populations of patients. For instance, drugs may be designed to regulate the biological activity of variants implicated in the disease process while minimizing effects on other variants.

C. Detection of Polymorphisms

As described above, detection of HTR1D, OPRD1, DRD2 or other polymorphisms of the invention generally comprises the step of determining at least part of the sequence of an HTR1D, OPRD1, DRD2 or other gene in a sample, preferably a patient sample, at one or more of the positions herein described.

Any analytical procedure may be used to detect the presence or absence of variant nucleotides at one or more polymorphic positions of the invention. In general, the detection of allelic variation requires a mutation discrimination technique, optionally an amplification reaction and optionally a signal generation system. Many current methods for the detection of allelic variation are reviewed by Nollau et. al. (1997), Clin. Chem. 43: 1114-1120; and in standard textbooks, for example, Laboratory Protocols for Mutation Detection by U. Landegren, Oxford University Press, 1996 and PCR, 2nd Edition by Newton & Graham, BIOS Scientific Publishers Limited, 1997.

Any means of mutation detection or discrimination may be used. For instance DNA sequencing, scanning methods, hybridization, extension-based methods, incorporation-based methods, restriction enzyme-based methods and ligation-based methods may be used in the methods of the invention. Sequencing methods include, but are not limited to, direct sequencing and sequencing by hybridization. Scanning-methods include, but are not limited to, protein truncation test (PTT), single-strand conformation polymorphism analysis (SSCP), denaturing gradient gel electrophoresis (DGGE), temperature gradient gel electrophoresis (TGGE), cleavase, heteroduplex analysis, chemical mismatch cleavage (CMC), and enzymatic mismatch cleavage.

Hybridization-based methods of detection include, but are not limited to, solid phase hybridization such as dot blots, multiple allele specific diagnostic assay (MASDA), reverse dot blots, and oligonucleotide arrays (DNA Chips). Solution phase hybridization amplification methods may also be used, such as Taqman®.

Extension based methods include, but are not limited to, amplification refractory mutation system (ARMS), amplification refractory mutation system linear extension (ALEX), and competitive oligonucleotide priming system (COPS).

Incorporation-based detection methods include, but are not limited to, mini-sequencing and arrayed primer extension (APEX). Restriction enzyme-based detection systems include, but are not limited to, RFLP, and restriction site generating PCR. Lastly, ligation based detection methods include, but are not limited to, oligonucleotide ligation assay (OLA).

Signal generation or detection systems that may be used in the methods of the invention include, but are not limited to, fluorescence methods such as fluorescence resonance energy transfer (FRET), fluorescence quenching, fluorescence polarization as well as other chemiluminescence, electrochemiluminescence, Raman, radioactivity, calorimetric methods, hybridization protection assay and mass spectrometry.

Further amplification methods include, but are not limited to self sustained replication (SSR), nucleic acid sequence based amplification (NASBA), ligase chain reaction (LCR), strand displacement amplification (SDA) and branched DNA (b-DNA).

D. Nucleotide Primers and Probes

The invention further provides nucleotide primers which can detect the polymorphisms of the invention. In one embodiment of the invention, primers are prepared that are capable of detecting an HTR1D, OPRD1, DRD2, or other gene polymorphism at one or more of the positions herein described. Preferred primers allow detection of an HTR1D, OPRD 1, DRD2, or other polymorphism associated with an eating disorder, such as a polymorphism in an HTR1D, OPRD1, or DRD2 gene corresponding to the polymorphisms in the Tables as described herein.

Allele specific primers are typically used together with a constant primer, in an amplification reaction such as a PCR reaction, which provides the discrimination between alleles through selective amplification of one allele at a particular sequence position. The allele specific primer is preferably about 10, 12, 15, 17, 19 or up to about 50 or more nucleotides in length, more preferably about 17-35 nucleotides in length, and more preferably about 17-30 nucleotides in length.

The allele specific primer preferably corresponds exactly with the allele to be detected but allele specific primers may be derivatives wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example: Protocols for Oligonucleotides and Analogues; Synthesis and Properties, Methods in Molecular Biology Series; Volume 20; Ed. Sudhir Agrawal, Humana ISBN: 0-89603-247-7; 1993; 1st Edition. If required, the primer(s) may be labeled to facilitate detection.

The invention also provides allele-specific probes that are capable of detecting an HTR1D, OPRD1, DRD2 or other polymorphism associated with an eating disorder. Preferred probes allow detection of an HTR1D, OPRD1, DRD2 or other polymorphism associated with an eating disorder, such as a polymorphism in an HTR1D, OPRD1, or DRD2 gene corresponding to the polymorphisms designated in the Tables. The primers and probes of the invention will preferably be labeled at their 3′ and 5′ ends, more preferably labeled at the 5′ end with ZipCode™ sequences (Ye et al. 2001, Hum. Mutat. 17: 305-316).

Such probes are of any convenient length, such as up to about 50 bases or more, up to 40 bases, and more conveniently up to 30 bases in length, such as for example 8-25 or 8-15 bases in length. In general such probes will comprise base sequences entirely complementary to the corresponding wild type or variant locus in the gene. However, if required, one or more mismatches may be introduced, provided that the discriminatory power of the oligonucleotide probe is not unduly affected. Such probes can also be up to about 80 bases or more, such that a mismatch will disrupt the hybridization characteristics of the oligonucleotide probe. The probes of the invention may carry one or more labels to facilitate detection.

According to another aspect of the present invention there is provided a diagnostic kit comprising at least one allele specific oligonucleotide probe or primer of the invention and/or an allele-nonspecific primer of the invention. The diagnostic kits may comprise appropriate packaging and instructions for use in the methods of the invention. Such kits may further comprise appropriate buffer(s), nucleotides, and polymerase(s) such as thermostable polymerases, for example Taq polymerase. The probes or primers may optionally be attached to a solid support.

The present invention also includes a computer readable medium comprising at least one novel polynucleotide sequence of the invention stored on the medium, such as a nucleotide sequence spanning a polymorphisin in an HTR1D, OPRD1, DRD2 or other gene as herein described. The computer readable medium may be used, for example, in homology searching, mapping, haplotyping, genotyping or pharmacogenetic analysis or any other bioinformatic analysis.

The polynucleotide sequences of the invention, or parts thereof, particularly those relating to and identifying the single nucleotide polymorphisms identified herein represent a valuable information source, for example, to characterize individuals in terms of hap1otype and other sub-groupings, such as investigating the susceptibility to treatment with particular drugs. These approaches are most easily facilitated by storing the sequence information in a computer readable medium and then using the information in standard bioinformatics programs or to search sequence databases using state of the art searching tools. Thus, the polynucleotide sequences of the invention are particularly useful as components in databases useful for sequence identity and other search analyses. As used herein, storage of the sequence information in a computer readable medium and use in sequence databases in relation to “polynucleotide or polynucleotide sequence of the invention” covers any detectable chemical or physical characteristic of a polynucleotide of the invention that may be reduced to, converted into or stored in a tangible medium, such as a computer disk, preferably in a computer readable form. For example, chromatographic scan data or peak data, photographic scan or peak data, mass spectrographic data, sequence gel (or other) data may be included.

A computer based method is also provided for performing sequence identification, said method comprising the steps of providing a polynucleotide sequence comprising a polymorphism of the invention in a computer readable medium; and comparing said polymorphism containing polynucleotide sequence to at least one other polynucleotide or polypeptide sequence to identify identity (homology), i.e., screen for the presence of a polymorphism.

E. Methods to Identify Agents that Modulate the Expression of HTR1D, OPRD1, DRD2 or Other Genes

Another embodiment of the present invention provides methods for identifying agents that modulate the expression of a nucleic acid encoding an HTR1D, OPRD1, DRD2, or other gene variant of the invention. Such assays may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, the expression of a nucleic acid encoding an HTR1D, OPRD1, DRD2, or other gene of the invention in a cell or tissue sample is monitored directly by hybridization to the nucleic acids of the invention. Cell lines or tissues are exposed to the agent to be tested under appropriate conditions and time and total RNA or mRNA is isolated by standard procedures such those disclosed in Sambrook et al., (1989) Molecular Cloning—A Laboratory Manual, Cold Spring Harbor Laboratory Press).

Probes to detect differences in RNA expression levels between cells exposed to the agent and control cells may be prepared as described above. Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA RNA from a sample under conditions in which the affixed sequences will specifically hybridize to the RNA. By examining for the ability of a given probe to specifically hybridize to an RNA sample from an untreated cell population and from a cell population exposed to the agent, agents which up or down regulate expression are identified.

F. Methods to Identify Agents that Modulate the Levels or at Least One Activity of an HTR1D, OPRD1, DRD2 or Other Gene Product

Another embodiment of the present invention provides methods for identifying agents that modulate the cellular level or concentration or at least one activity of a protein of the invention. Such methods or assays may utilize any means of monitoring or detecting the desired activity.

In one format, the relative amounts of a protein of the invention between a cell population that has been exposed to the agent to be tested compared to an un-exposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes are prepared by immunizing suitable mammalian hosts in appropriate immunization protocols using the peptides, polypeptides or proteins of the invention if they are of sufficient length, or, if desired, or if required to enhance immunogenicity, conjugated to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co. (Rockford, Ill.), may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein (Nature (1975) 256: 495-497) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonals or the polyclonal antisera which contain the immunologically significant portion can be used as antagonists, as well as the intact antibodies. Use of immunologically reactive fragments, such as the Fab, Fab′, of F(ab′)₂ fragments is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin, such as humanized antibodies.

Agents that are assayed in the above methods can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, nucleic acid molecules such as antisense molecules that specifically recognize a variant delta opioid receptor as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Grant in: Meyers (ed.) Molecular Biology and Biotechnology (New York, VCH Publishers, 1995), pp. 659-664). A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

G. Solid Supports

Solid supports containing oligonucleotide probes for identifying the SNPs of the present invention can be filters, polyvinyl chloride dishes, silicon or glass based chips, etc. Such wafers and hybridization methods are widely available, for example, those disclosed by Beattie (WO 95/11755). Any solid surface to which oligonucleotides can be bound, either directly or indirectly, either covalently or noncovalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, about 2, 10, 100, 1000 to 10,000; 100,000, 400,000 or 1,000,000 of such features on a single solid support. The solid support, or the area within which the probes are attached may be on the order of a square centimeter.

Oligonucleotide probe arrays can be made and used according to any techniques known in the art (see for example, Lockchart et al. (1996), Nat. Biotechnol. 14: 1675-1680; McGall et al. (1996), Proc. Nat. Acad. Sci. USA 93: 13555-13460). Such probe arrays may contain at least two or more oligonucleotides that are complementary to or hybridize to two or more of the SNPs described herein. Such arrays may also contain oligonucleotides that are complementary or hybridize to at least about 2, 3, 4, 5, 6, 7, 8, 9, 10, 20, 30, 50 or more SNPs described herein.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see Pirrung et al. (1992), U.S. Pat. No. 5,143,854; Fodor et al. (1998), U.S. Pat. No. 5,800,992; Chee et al. (1998), U.S. Pat. No. 5,837,832.

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in Fodor et al., (1993). WO 93/09668. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

H. Databases

The present invention includes databases containing information concerning SNPs associated with eating disorders, for instance, information concerning SNP allele frequency and strength of the association of the allele with an eating disorder and the like. Databases may also contain information associated with a given polymorphism such as descriptive information about the probability of association of the polymorphism with a specific eating disorder. Other information that may be included in the databases of the present invention include, but is not limited to, SNP sequence information, descriptive information concerning the clinical status of a tissue sample analyzed for SNP haplotype, or the subject from which the sample was derived. The database may be designed to include different parts, for instance a SNP frequency database and a SNP sequence database. Methods for the configuration and construction of databases are widely available, for instance, see Akerblom et al., (1999) U.S. Pat. No. 5,953,727, which is herein incorporated by reference in its entirety.

The databases of the invention may be linked to an outside or external database. In a preferred embodiment, the external database may be the HGBASE database maintained by the Karolinska Institute, The SNP Consortium (TSC) and/or the databases maintained by the National Center for Biotechnology Information (NCBI) such as GenBank.

Any appropriate computer platform may be used to perform the necessary comparisons between SNP allele frequency and associated disorder and any other information in the database or provided as an input. For example, a large number of computer workstations are available from a variety of manufacturers, such as those available from Silicon Graphics. Client-server environments, database servers and networks are also widely available and appropriate platforms for the databases of the invention.

The databases of the invention may also be used to present information identifying the SNP alleles in a subject and such a presentation may be used to predict the likelihood that the subject will develop an eating disorder. Further, the databases of the present invention may comprise information relating to the expression level of one or more of the genes associated with the SNPs of the invention.

The SNPs identified by the present invention may be used to analyze the expression pattern of an associated gene and the expression pattern correlated to the probability of developing an eating disorder. The expression pattern in various tissues can be determined and used to identify tissue specific expression patterns, temporal expression patterns and expression patterns induced by various external stimuli such as chemicals or electromagnetic radiation.

Without further description, it is believed that one of ordinary skill in the art can, using the preceding description and the following illustrative examples, make and utilize the compounds of the present invention and practice the claimed methods. The following working example therefore, specifically point out preferred embodiments of the present invention, and are not to be construed as limiting in any way the remainder of the disclosure.

### EXAMPLES

### Example 1

### Identification of Subjects and Controls

1. Subjects

AN-ARP Database

Probands (n=196) were participants in a multicenter study aimed at identifying genes involved in eating disorders, and related traits. All probands met the DSM-IV criteria for a lifetime AN diagnosis (DSM-IV definition, 1994). The probands are composed of both DSM-IV AN1, restricting subtype, and AN2, purging subtype (55% and 45%, respectively). Other requirements for study participation were that the women were aged between 13-65, age of onset before 25, and fulfillment of the criteria of AN for at least 3 years prior to ascertainment. The probands had a minimum past BMI of 14.27+/−2.88. 182 parents of probands and 260 affected relatives were also included. Probands, parent and affected relatives were recruited in the same study, where the affected relative fulfilled American Psychiatry Association criteria for AN, BN, and eating disorders otherwise specified. A detailed description of sample and methods can be found in Kaye et al. (2000).

BN-ARP Database

The BN-ARP dataset is comprised of probands and affected relatives. All probands met DSM-IV criteria for BN with a minimum 6 month period of binging and vomiting at least twice a week. Some had an additional lifetime history diagnosis of AN (BN+AN). All affected relatives met DSM-IV criteria for BN, AN, BN with a lifetime history of AN (BN+AN), or eating disorder NOS. The methods were similar to the PF AN ARP study with the addition of SCID I and II assessments. Assessments were obtained from 187 BN probands and 194 BN+AN probands (this figure includes probands with both diagnoses). There were 346 probands with DNA available for genotyping. Overall, there were 378 relative pairs available for linkage analysis. Of the BN proband-relative pairs, the following diagnoses were reported: 33.7% BN, 21.4% BN+AN, 25.1% AN, and 18.2% eating disorder NOS. Of the BN+AN proband-relative pairs, the following diagnoses were reported: 22.2% BN, 25.3% BN+AN, 36.1% AN, and 16.9% eating disorder NOS. 50 cc's of blood were collected on each subject. The BN-ARP dataset excluded any proband with only ANR or ANRP. (Probands could have additional diagnoses of ANR or ANRP. They just could not have those as exclusive diagnoses.)

2. Control Samples

A control sample of European-American female sample (EAF, n=98) was recruited through advertisements. This sample was screened to exclude obese individuals (>20% ideal weight), as well as the presence of lifetime criteria for Axis I disorders assessed by Structured Clinical Interview for DSM-III-R (SCID) criteria (Sheehan et al. (1997), European Psychiatry 12: 232-241). Unrelated Centre Etude Polymorphism Humaine DNA samples (“CEPH”) obtained from Coriell Cell Repositories were used for resequencing, for genotype assay development and for sequence verification of the homozygosity status of individual control DNAs.

### Example 2

### Molecular Genetic Methods

1. Sequence Evaluation and Annotation

Evaluation of cDNA and genomic sequence and sequence variation was accomplished using public sequence and variation databases. Aligmnent and annotation of genomic and cDNA sequences was accomplished using the Sequencher™ sequence evaluation package version 4.0.5 (Gene Codes Corporation, Ann Arbor, Mich.). Polymorphisms (Table 1) at the HTR1D, OPRD1, and DRD2 loci were identified by examination of variation databases including NCBI (dbSNP, http://ncbi.nlm.nih.gov; HGBase, http://hgbase.cgr.ki.se/), The SNP Consortium (TSC, http://snp.cshl.org/) and resequencing within the Biognosis laboratory.

In the present study, a large number of candidate genes were screened for polymorphisms for genotyping in AN association analyses. The sequencing results are summarized in table below. A summary of the candidate gene polymorphisms that were genotyped can be found in Table 1.

| | | BP per | Total N | Number of |
|---|---|---|---|---|
| Gene name | Gene symbol | individual | sequenced | SNPs identified |
| β1-adrenergic receptor | ADRB1 | 617 | 32 | 0 |
| Catechol-O-methyltransferase | COMT | 513 | 32 | 1 |
| Cocaine - and amphetamine- | CART | 1853 | 32 | 2 |
| regulated transcript | | | | |
| Corticotropin-releasing hormone | CRH | 617 | 32 | 0 |
| Dopamine receptor D2 | DRD2 | 537 | 32 | 2 |
| Glucagon | GCG | 3896 | 32 | 2 |
| Hypocretin (orexin) neuropeptide | HCRT | 2504 | 32 | 0 |
| precursor | | | | |
| Hypocretin receptor 1 | HCRTR1 | 5386 | 64 | 13 |
| Hypocretin receptor 2 | HCRTR2 | 6276 | 32 | 2 |
| Melanin concentrating hormone | MCH | 1993 | 64 | 0 |
| Melanocortin-3 receptor | MC3R | 1920 | 32 | 2 |
| Melanocortin-4 receptor | MC4R | 1806 | 32 | 1 |
| Neuropeptide Y | NPY | 7337 | 166 | 20 |
| δ Opioid receptor | OPRD1 | 4180 | 32 | 1 |
| Serotonin receptor 1A | HTR1A | 3408 | 64 | 3 |
| Serotonin receptor 1D | HTR1D | 4400 | 64 | 3 |
| Serotonin receptor 1E | HTR1E | 863 | 32 | 1 |
| Serotonin receptor 1F | HTR1F | 1037 | 32 | 0 |
| Tryptophan hydroxylase | TPH | 3786 | 32 | 7 |

2. Resequencing

Primers for HTR1D and OPRD1 are listed in Table 2. The general PCR conditions for sequencing were (per 50 μL reaction): 50 ng genomic DNA, 25 nM each of the forward and reverse primers, 10 mM dNTP, 50 mM MgCl₂, 160 mM (NH₄)₂SO₄, 670 mM Tris-Cl (pH 8.8 at 25° C.), 0.1% Tween-20, and 2.5U Taq DNA polymerase (Bioline, Springfield, N.J.). General conditions for the PCR were: 94° C. for 1 min, followed by 30 cycles of 94° C. for 15 s, Tₐ° C. for 30 s (where Tₐ° C. was 65° C. for all primer pairs with the following exceptions: 62° C. for PF-0075/PR-0074, PF-0010/PR-0011 and PF-0087/PR-0088 and 55° C. for PF-0078/PR-0079), 72° C. for 1 min, with a final extension step of 72° C. for 5 min.

Post-PCR, 50 μl of each product was purified by Millipore PCR Purification System (Millipore Corporation, Danvers, Mass.). Products were then requantitated (O.D. at 260 nm) and 0.25 μg product was mixed with 25 pM primer, 4 μl Big Dye Terminators (PE Biosystems, Foster City, Calif.) with the volume brought to 20 μl with water. Cycle sequencing was performed using the following PCR conditions: 96° C. for 5 minutes, followed by 35 cycles of 96° C. for 10 sec, 50° C. for 10 sec, and 60° C. for 3.5 min, with a 4° C. hold.

3. Genotyping

DRD2 Genotyping

The number of AN probald DNA samples subjected to genotyping at DRD2-43, DRD2-11, DRD2-23, DRD2-24, DRD2-25, DRD2-35, and DRD2-42 were N=183, 132, 183, 191, 191, 132, and 132, respectively. The number of AN-ARP family members subjected to genotyping at DRD2-43, DRD2-23, DRD2-24, and DRD2-25 was N=457. The number of EAF DNA sample genotypes subjected to genotyping at DRD2-43, DRD2-11, DRD2-24, DRD2-25, DRD2-35, and DRD2-42 was N=98. A sample of 67 duplicate DNA samples from all subgroups was genotyped to assess the reproducibility of the DRD2-43, DRD2-11, DRD2-24, DRD2-25, DRD2-35, and DRD2-42 SNP genotyping assays. DRD2-43, DRD2-23, DRD2-24, and DRD2-25 genotypes were evaluated for apparent non-Mendelian transmissions. DRD2 SNPs were genotyped using 5′ exonuclease assay (TaqMan™) (Morin et al., 1999), with the exception of DRD2-43, which was typed as described (Arinami et al. (1997), Hum. Mol. Genet. 6: 577-582), and DRD2-23, which was genotyped as described (Fujiwara et al. (1997), Eur. Neurol. 38: 6-10).

5′ exonuclease probes and primers were chosen using ProbeITY (Celadon Laboratories, College Park, Md.) and were synthesized by Applied Biosystems (Foster City, Calif.). A verification plate consisting of 17% of the AN probands and control group samples was genotyped order to assess the reproducibility of the assay. Other quality control procedures in the laboratory included no template controls for genotype assay quality control. Primer and probe sets are as follows: DRD2-11: forward primer —5′-AGCAGAGGAAGGAGTG-3′ (SEQ ID NO: 4), reverse primer —5′-AATGATGCCTGGATGC-3′ (SEQ ID NO: 5), probe 1—FAM-tccctagtcAaacccaaggct-TAMRA (SEQ ID NO: 6), probe 2—TET-tcctagtcGaacccaaggc-TAMRA (SEQ ID NO: 7); DRD2-24: forward primer—5′-CTGACTCTCCCCGAC-3′ (SEQ ID NO: 8), reverse primer—5′-CTTGGGGTGGTCTTTG-3′ (SEQ ID NO: 9), probe 1-FAM-ccaccaCggtctccacggc-TAMRA (SEQ ID NO: 10), probe 2-VIC-ccaccaTggtctccacggc-TAMRA (SEQ ID NO: 11); DRD2-25: forward primer—5′-CCCATTCTTCTCTGGTTT-3′ (SEQ ID NO: 12), reverse primer—5′-CTGACTCTCCCCGAC-3′ (SEQ ID NO: 13), probe 1-FAM-cggggctgtcAggagtgc-TAMRA (SEQ ID NO: 14), probe 2-VIC-cggggctgtcGggagt-TAMRA (SEQ ID NO: 15); DRD2-35: forward primer—5′-TATGGGGAGAGGAACTC-3′ (SEQ ID NO: 16), reverse primer—5′-GAGAAGGGATACATTGCA-3′ (SEQ ID NO: 17), probe 1—FAM-ageccaccctGctgcc-TAMRA (SEQ ID NO: 18), probe 2—TET-agcccaccctTctgcctt-TAMRA (SEQ ID NO: 19); DRD2-42: forward primer—5′-CAACACAGCCATCCTC-3′ (SEQ ID NO: 20), reverse primer—5′-TCACTCCATCCTGGAC-3′ (SEQ ID NO: 21), probe 1—FAM-ctggtcAaggcaggctc-TAMRA (SEQ ID NO: 22), probe 2—VIC-tggtcGaggcaggcgc-TAMRA (SEQ ID NO: 23). General conditions per reaction for PCR and endpoint-read TaqMan™ were as described (Morin et al. (1999), Biotechniques 27: 538-540, 542, 544), with the exception of DRD2-24 and -25; PCR reaction conditions were optimized with an annealing temperature of 60° C. Genotype discrimination was conducted manually by a technician on an Applied Biosystems Sequence Detector 7700 (Applied Biosystems, Foster City, Calif.).

HTR1D and OPRD1 Genotyping

For each gene, multiple SNPs were selected for genotyping using the 5′ exonuclease assay using the criteria of location, allele frequency and polymorphism effect. Probes and primers were chosen using ProbeITY (Celadon Laboratories, College Park, Md.) and were synthesized by Applied Biosystems (Foster City, Calif.). General conditions for endpoint-read TaqMan™ PCR were as described (Morin et al. (1999), Biotechniques 27: 538-540), except for OPRD1(47821A>G), where 300 nM of each of the 2 probes, and for HTR1D(-1123T>C), where 300 nM of the TET probe was used. See Table 5 for primer and probe sequences used in these TaqMan genotyping and sequencing assays. Genotype determination was conducted manually by a technician using Applied Biosystems software on the Applied Biosystems Sequence Detector 7700 (Applied Biosystems, Foster City, Calif.). For each assay, 653 AN probands, affected siblings, and other family members were genotyped, as well as an additional 244 control samples from different sources. A verification plate consisting of 17% of the AN probands and control group samples was genotyped in order to assess the reproducibility. of the assay. Quality control procedures in the laboratory included genotyping of a duplicated sample (N=72) to assess genotyping error rate, no template (no genomic DNA) controls for genotype assay quality control and Hardy-Weinberg equilibrium (HWE) tests for overall genotype error checking. Observed discordant genotypes were dropped from analysis.

### Example 3

### Statistical Procedures and Analyses

1. Statistical Procedures

Table 12 presents the data for the TDT analyses performed at polymorphisms typed in the AN-ARP probands and parents, where results for one allele are present, except in cases where the other allele gives a different result (OPRD 1-07) and where there are more than two alleles (DAT, DRD4).

Association analysis to DSM-IV AN diagnosis using contingency table analysis (χ² and Fisher tests) of genotype and allele counts was performed at seventy-three DNA polymorphisms (N=73) at twenty-nine (N=29) candidate gene. For each SNP, association between genotypic or allelic counts at a candidate gene polymorphism and DSM-IV AN or an orexia subtype was performed. The phenotypes used in the various analyses were: DSM-IV 307.1 or AN, also restricting subtype, referred to as AN1, also purging subtype, referred to as AN2 and DSM-IV 307.51or BN. For each SNP, six (N=6) tests of association are reported (AN-1, AN-2, and all AN versus the EAF control samples) using genotype counts (3 tests) and allelic counts (3 tests). Summary results of these contingency analyses for the AN-ARP dataset are presented in Table 19.

Because the BN-ARP dataset dxcode hierarchy—subtype—was not available at the time of analysis, BN-ARP proband status as “caseness” was used and contingency table analysis was performed using genotypes and allele (2 tests). A summary of results of these contingency analysis from the BN-ARP dataset are presented in Table 20.

Transmission disequilibriun analysis of SNP association to DSM-IV AN diagnosis using AN-ARP probands and parents using the TDT test was performed at thirty-nine (N=39) DNA polymorphisms at eighteen (N=18) candidate genes and a summary of results is available in Table 12. SNPs that exhibited significance at the alpha 0.05 or 0.10 level are included in Table 19.

Transmission disequilibrium analysis of SNP association to all DSM-IV eating disorder diagnoses using the TDT test was performed at thirty-one (N=31) SNPs at twelve (N=12) candidate genes in the BN-ARP dataset. Table 22 shows the results of a TDT analysis in BN probands and parents only. TDT analysis of SNP association to BN-ARP proband status (DSM-IV BN probands) was performed at thirty-one (N=31) SNPs at twelve (N=12) candidate genes in the BN-ARP dataset. Table 23 shows the results of the analysis for the entire BN-ARP dataset.

TDT Analysis of AN-ARP Dataset

On the data from a cleaned muaster dataset-apparenit non-Mendelian transmissions removed-Spiehnan's TDT (http://genomics.med.upenn.edu/spielman/TDT.htm) was used for transmission disequilibrium analysis tests. Table 21 presents data for six polymorphisms from 5 genes that exhibited statistically significant transmission disequilibrium:

1) One SNP-5HTT-06-at the serotonin transporter gene (ch. 17q11.1-q12), Z=2.041, p=0.041.

2) One SNP-ADRB2-01-at the β2-adrenergic receptor gene (ch. 5q31-q32), Z=1.960, p=0.050.

3& 4) Two SNPs-DRD2-25 and DRD2-43-at the dopamine receptor D2 gene (ch. 11q23), DRD2-25: Z=2.604, p=0.009, and DRD2-43: Z=2.582, p=0.010.

5) One SNP-DRD3-01-at the dopamine receptor D3 gene (ch. 3q13.3), Z=2.635, p=0.008.

6) One SNP-HTR1D-03-at the serotonin 1D receptor gene (ch. 1p36.3-p34.3), Z=2.000, p=0.046.

TDT Analysis of BN-ARP Database

Two different transmission disequilibrium (TDT) analyses, were performed to determine whether there are different effects between proband status and other eating disorders (in the ARPs). TDT analyses were performed in two ways: 1.) on the entire BN-ARP dataset (N˜929) and 2.) on the probands and their parents only (N˜528). Results are presented in Tables 22 and 23 respectively. The proband/parent TDT analyses were performed using FBAT (http://www.biostat.harvard.edu/˜fbat/default.html) and the entire ARP dataset was analyzed using S.A.G.E. (TDTEX).

Five SNPs at 4 genes showed statistically significant transmission disequilibrium in the BN-ARP dataset (Table 22). Table 22 presents data from 4 different tests of TDT, Permutation McNemar, Asymptotic McNemar, Asymptotic Marginal, and Permutation Marginal for alleles and genotypes (8 tests total per SNP). The values reported are the p values from each test, with standard errors when applicable.

1) There was a very marginal TDT result a SNP-CCK-01-at the cholecystokinin gene (ch. 3p22-p21.3) for only one of the 8 tests (alleles: Permutation Marginal, p=0.05).

2 &3) Two SNPs-DRD2-11 and DRD2-24at the dopamime receptor D2 gene (ch.11q23) showed significance for TDT. Four of the 8 tests were significant for DRD2-11 (alleles: Permutation McNemar, p=0.019; Asymptotic McNemar, p=0.015; Asymptotic Marginal, p=0.015; and Permutation Marginal, p=0.020). For DRD2-24, all 8 tests reported significant transmission disequilibrium (alleles: Permutation McNemar, p=0.009; Asymptotic McNemar, p=0.007; Asymptotic Marginal, p=0.007; and Permutation Marginal, p=0.010; genotypes: Permutation McNemar, p=0.021; Asymptotic McNemar, p=0.026; Asymptotic Marginal, p=0.008; and Permutation Marginal, p=0.008. This gene has previously shown evidence for association with AN (Biognosis, Bergen et al., in preparation).

4) One SNP-HTR1B-03-at the serotonin 1B receptor gene (ch. 6q13) showed significance for TDT. Five of the 8 tests reported significant transmission disequilibrium: (alleles: Asymptotic McNemar, p=0.048; Asymptotic Marginal, p=0.048; genotypes: Permutation McNemar, p=0.008; , Asymptotic McNemar, p=0.012; Permutation Marginal, p=0.047). Note that two other SNPs at this gene showed evidence for case: control association (HTR1B-01 and -02).

5) One SNP-HTR2A-18-at the serotonin receptor 2A gene (ch. 13q14-q21) showed very significant TDT results. For HTR2A-18, each of the 8 tests produced significant TDT results: (alleles: Permutation McNemar, p 0.007; Asymptotic McNemar, p=0.005; Asymptotic Marginal, p=0.005; and Permutation Marginal, p=0.007; genotypes: Permutation McNemar, p=0.016; Asymptotic McNemar, p=0.023; Asymptotic Marginal, p=0.007; and Permutation Marginal, p=0.009).

Three SNPs at two genes showed statistically significant transmission disequilibrium in the BN proband/parent dataset (Table 23).

1 & 2) Two SNPs-DRD2-24 and DRD2-35-at the dopanmine receptor D2 gene (ch. 11q23) showed statistically significant transmission disequilibrium: DRD2-24 (Z=3.111; p=0.002) and DRD2-35 (Z=2.117; p=0.034). DRD2-24 is a silent mutation located in exon 7, while DRD2-35 is located 3′ of the gene (Table 23). Polymorphisms at DRD2 have been imuplicated in AN (see U.S. provisional patent application serial no. 60/331,285, filed Nov. 13, 2001).

3) One SNP-HTR2A-18-at the serotonin 2A receptor gene (ch. 13q14-q21) showed statistically significant transmission disequilibrium: HTR2A-18 (Z=2.982; p=0.003) (Table 23). This SNP is located 5′ of the gene.

DRD2 Statistical Analysis

Contingency table (χ²) analyses of genotype, allele, and haplotype counts were performed using SigmaStat (Jandel Corporation, San Rafael, Calif.). 95% confidence intervals were obtained using PROCFREQ in SAS. Spielman's TDT (http://genomics.med.upenn.edu/spielman/TDT.htm) was used for transmission disequilibrium analysis (Spielman et al. (1993), Am. J. Hum. Genet. 52: 506-516). Multi-locus genotypes at DRD2-43, -11, -24, -25, -35, -42 SNPs from one hundred twenty-five (N=125) AN proband and eighty-seven (N=87) EAF samples were assembled in a Visual Basic utility. Resulting multilocus genotype counts were tested for pairwise linkage disequilibrium using likelihood ratio tests with significance testing by permutation using Arlequin (http://lbg.unige.ch/arlequin/). An EM algorithm was used separately to estimate multi-locus haplotype frequencies. Pedcheck2 was used to identify apparent non-Mendelian transmissions in the AN-ARP family sample. HWE in the AN, AN1, and AN2 proband and EAF samples was evaluated using contingency table (χ²) analysis.

Table 6 shows the results of genotypic and allelic contingency (χ²) analyses for cases vs. controls for six DRD2 polymorphisms. There were statistically significant frequency differences between patients and controls at the DRD2-43-141 Indel SNP at both the genotypic and allelic levels (χ²=5.20, p=0.023 and χ²=4.77, p=0.029, respectively). The estimate of the risk associated with the two DRD2-43 -141 SNP alleles (Table 6) is a disease susceptibility risk for DRD2-43-141C (Allele 2) of 2.02 (95% confidence interval 1.07-3.83) and a protective odds ratio for DRD2-43-141Del (Allele 1) of 0.49 (95% confidence interval=0.26-0.94). Analysis of DRD2-43-141C by DSM-IV AN subtype (AN1 and AN2) revealed a statistically significant association with DSM-IV AN1 diagnosis p=0.039 and p=0.049 genotypewise and allelewise, respectively), but not with DSM-IV AN2 diagnosis (genotypes p=0.087, alleles p=0.104). In the AN2 sample, DRD2-42 exhibits a statistically significant association at the genotypic level (p=0.035), but not at the allelic level: None of the other DRD2 SNPs tested showed an association to DSM-IV AN when comparing genotypic or allele counts in the proband sample and the control sample.

HTR1D and OPRD1 Statistical Procedures

HWE was evaluated using contingency analysis. Multi-locus HTR1D and OPRD1 genotypes were assembled in a Visual Basic utility and resulting multilocus genotype counts were used to estimate intragenic and intergenic pairwise linkage disequilibrium using likelihood ratio tests with empirical significance testing (using 10,000-16,000 permutations) using Arlequin.

The significance of differences in genotype and allele frequencies at the HTR1D, OPRD1 and HCRTR1 loci between the AN and control samples was evaluated using chi-square (χ²) analysis in SAS. In case of expected cell frequency ≦5, the p-value was based on the Fisher exact test. The empirical significance of haplotype frequency differences between AN and control samples were evaluated using the nonparametric heterogeneity statistic (T5) in the program EH+ using 10,000 permutations (Zhao et al. (2000), Hum. Hered. 50: 133-139). In order to maximize the power to detect transmission disequilibrium at individual SNPs, FBAT was used for transmission disequilibrium analysis (TDT). (Horvath et al. (2001), Eur. J. Hum. Genet. 9: 301-306). A p value of <0.05 is described in all analyses as indicating a statistically significant result, while p values≧0.05 and <0.10 are described in all analyses to indicate a trend towards statistical significance.

DRD2 Linkage Disequilibrium

Statistically significant pairwise linkage disequilibrium (LD) was observed in both the AN proband and EAF samples among DRD2 SNPs (Table 7). The percentage of marker pairs in significant LD in the AN sample was 87%; in the EAF sample the percentage was 60%. The statistical significance of LD in the AN sample was substantially greater than in the EAF sample for all marker pairs in statistically significant LD. The physical extent of LD was greater in the AN sample than in the EAF sample. The SNPs tested (DRD2-43/11/24/25/35/42) span a region >270 kb. In the AN sample, two blocks of LD were observed; one involved the DRD2-43/11/24/25 SNPs (the 5′ block) spanning ˜260 kb, with the other overlapping region consisting of the DRD2-11/24/25/35/42 SNPs (3′ block) spanning ˜22 kb. LD was not statistically significant between the one extreme 5′ polymorphism DRD2-43) and the two most extreme 3′ polymorphisms (DRD2-35 and 42). In the EAF sample, there was not statistically significant LD between DRD2-43 and any of the 3′ SNPS (DRD2-11/24/25/35/42), though the EAF sample did have a similarly statistically significant 3′ LD block (DRD2-11/24/25/35/42) as the AN sample.

The LD observed in the present invention enables the results of association between DRD2 SNPs and AN in the case:control and family data to be interpreted as internally concordant, i.e., the same and different SNPs observed to be statistically significantly associated with AN in the case: control and family samples respectively are in statistically significant LD, providing internal concordance that would not be available is only one sample comparision type or single DRD2 polymorphisms were investigated.

HTR1D, OPRD1, and HCRTR1 Linkage Disequilibrium

HWE equilibrium (26 tests of HWE performed) was observed for all HTR1D, OPRD1 and HCRTR1 SNPs in the AN proband and EAF control samples (data not shown). A trend towards deviation from HWE was observed at OPRD1(80T>G) and HCRTR1(846A>G) in the AN proband sample only. Significant pairwise linkage disequilibrium was observed in both the AN proband and EAF samples among HTR1D and OPRD1 SNPs (AN Proband sample linkage disequilibrium shown in Table 9, EAF data not shown). HTR1D intragenic pairwise LD among all HTR1D SNP pairs in both the AN and EAF samples was complete, that is, only three of four expected haplotypes at each HTR1D SNP pair was observed. Significant intragenic LD among OPRD1 SNP pairs was observed at eight of ten OPRD1 SNP pairs in both AN proband and EAF samples, where three often OPRD1 SNP pairs were observed to be highly significantly associated (p<10⁻⁵) in the AN proband sample. The OPRD1(8214T>C)/OPRD1 (23340A>G) SNP pair was in complete linkage disequilibrium in both the AN and EAF samples (EAF data not shown). Significant linkage disequilibrium was observed at two of twenty intergenic HTR1D/OPRD1 SNP pairs in the AN sample (Table 9).

DRD2 Haplotype Analysis

Pairwise haplotype frequencies in AN proband samples and the control sample were estimated using maximum likelihood in order to compare haplotype frequencies between AN probands and controls. The average estimated two-locus haplotype counts were two hundred thirty (N=230) from the AN proband sample and one hundred fifty-four (N=154) from a the control sample, where these averages result from including only those individual probands or control individuals genotyped at the two DRD2 SNPs considered together. Contingency analysis of pairwise haplotype counts are shown in Table 24. The haplotype contingency results reveals the same pattern of association with AN phenotype, where association is observed to DRD2-43 where the most significant haplotype association to AN phenotype or subtype occurs in the AN1 sample with the DRD2-43/DRD2-24 haplotype (χ²=12.183, p=0.007).

The joint DRD2-43/DRD2-23 genotypes in the AN proband sample (N=7) were all observed to be DRD2-43 homozygotes/DRD2-23 C/G heterozygotes. Thus, all seven DRD2-23 G alleles were observed to be associated with the DRD2-43 C allele, the allele significantly over-represented in the AN and AN1 samples. Inspection of the DRD2-11, DRD2-24, and DRD2-25 genotypes in the seven AN probands with DRD2-43 C/C and DRD2-23 C/G genotypes conditional on the observed DRD2-43/DRD2-11/DRD2-24/DRD2-25 genotype frequencies suggests that the DRD2-43 C/DRD2-23 G haplotype allele has a DRD2-11 allele 2, DRD2-24 allele 2, DRD2-25 allele 2 configuration.

Association of HTR1D, OPRD1 and HCRTR1 SNPs to DSM-IV AN

Statistically significant association of HTR1D and OPRD1 SNPs to AN phenotype was observed at one HTR1D SNP, HTR1D(1080C>T), both genotypic and allelic, and at three of five OPRD1 SNPs, OPRD1(8214T>C), allelic, OPRD1(23340A>G), allelic, and OPRD1(47821A>G), both genotypic and allelic (Table 13). A trend towards significant association was observed at two HTR1D SNPs, HTR1D(2190A>,G), allelic and HTR1D(-628T>C), genotypic and at one OPRD1 SNP, OPRD1(51502A>T), genotypic. Note that removal of the males (N=10) in the AN proband sample, which results in entirely female case and control samples, increases the significance of all statistical tests by up to a factor of two, with a small increase in the risk effect of individual alleles (data not shown).

Significant HTR1D SNP haplotype frequency heterogeneity (Table 14) between the AN proband and EAF samples is observed with haplotypes containing the HTR1D(1080C>T) SNP, but not with the HTR1D haplotype containing all four SNPs. Significant OPRD1 SNP haplotype frequency heterogeneity between the AN proband and EAF samples is observed with OPRD1 SNP haplotype (8214T>C)/(47821A>G) and with the SNP haplotype containing all five SNPs. A trend towards significant haplotype frequency heterogeneity is observed with the remaining OPRD1 SNP haplotypes containing the 47821A>G SNP.

The same unrelated probands used in the case:control analyses described above were used as affected children for transmission disequilibrium analysis (Table 15). The average number of parental DNAs available for molecular genetic analysis is less than one parent per proband, limiting the number of trios available for analysis. Nevertheless, we observed significant transmission disequilibrium at three HTR1D SNPs, and a trend towards significant transmission disequilibrium at two OPRD1 SNPs.

Analyses Performed on the BN Dataset

Case: control contingency analyses were performed using BN proband (N˜346) vs. EAF (N˜89) samples. Since no DX code hierarchy was available, case: control was evaluated using the case status of the proband status only. Two different TDT analyses were performed to determine whether association differed between proband status and other eating disorders in the affected relative pairs in the BN-ARP dataset. The TDT was performed 1) for the entire ARP dataset, treating all eating disorder diagnoses as affected, and 2) on the probands and their parents only. The entire ARP dataset was analyzed using S.A.G.E. (TDTEX) and the proband/parent TDT analyses were performed using FBAT (http://www.biostat.harvard.edu/˜fbat/default.html). Six SNPs at 4 genes, ADRB3, ESR1, HTR1B, and HTR1D, showed a statistically significant association with BN proband status versus the EAF control sample at the, genotypic and/or allelic levels. See Table 16 for all case: control results.

Four SNPs at four genes showed statistically significant transmission disequilibrium in the entire BN-ARP dataset. Table 17 presents the p value and standard error from the Permuation McNemar TDT test statistic for all SNPs evaluated with the TDT and the four allelic TDT test statistics for those SNPs for which one of the four allelic TDT test statistics gave a result at the p<0.10 or better. Two SNPs at the DRD2 gene, DRD2-11 and DRD2-24, one SNP at the HTR1B gene, HTR1B-03, and one SNP at the HTR2A gene, HTR2A-18, showed significance for TDT. Two SNPs at the DRD2 gene, DRD2-24 and DRD2-35, and one SNP at the HTR2A gene, HTR2A-18, showed statistically significant transmission disequilibrium in the BN proband/parent dataset (see Table 18).

Case: control contingency analyses were performed using BN (N˜346) vs. XXF (N˜89) samples. Since no DX code hierarchy was available, case: control was evaluated using the inclusion due to proband status only.

Six SNPs at 4 genes showed a statistically significant association with BN proband status versus the XXF control sample at the genotypic and/or allelic levels.

1) One SNP-ADRB3-01-at the β3-adrenergic receptor gene (ch. 8p12-p11.2) was associated at both the genotypic (χ²=9.282; p=0.010) and allelic (χ²=9.422; p=0.009) levels. This nonsynonymous polymorphism, W64R, has previously been associated with Hyperinsulinaemia. One other polymorphism was typed at this gene in this dataset, but did not show significant association with BN.

2) One SNP-ESR-02-at the estrogen receptor 1 (α) gene (ch. 6q25.1) was associated at both the genotypic (χ²=11.179; p=0.004) and allelic (χ²=9.366; p=0.009) levels. There is nominally significant evidence for an association between the estrogen receptor 2 (β) gene (14q) and AN in the literature (Rosenkanz et al., J Clin Endocrinol Metab, 83: 4524-7, 1998).

3 & 4) Two SNPs-HTR1B-01 and HTR1B-02-at the serotonin 1B receptor gene (ch. 6q13) were associated with BN. HTR1B-01 was associated at both the genotypic (χ²=8675; p=0.013) and allelic (χ²=8.981; p=0.011) levels, as was HTR1B3-02 (genotype: χ²=7.493; p=0.024; allele: χ²=7.414; p=0.025). Both of these polymorphisms are silent mutations. Two other SNPs at this gene were typed in the BN dataset but did not show significant association with BN. There is a report of an association between HTR1B and BMI in BN women in the literature (Levitan et al. (2001), Biol. Psychiatry, 50: 640-643).

5 & 6) Two SNPs-HTR1D-02 and HTR1D-03-at the serotonin 1D receptor gene (ch. 1p36.3-p34.3) were associated with BN. HTR1D-02 was only associated with BN at the genotypic level (χ²=7.990; p=0.018). HTR1D-03 showed an association at both the genotypic (χ²=13.084; p=,0.001) and allelic (χ²=10.535; p=0.005) levels. Polymorphisms at this gene were previously found to be statistically significantly associated with AN (Bergen et al., submitted). Two additional polymorphisms were typed at this gene, neither of which show significant association with BN.

Although the present invention has been described in detail with reference to examples above, it is understood that various modifications can be made without departing from the spirit of the invention. Accordingly, the invention is limited only by the following claims. All cited patents, patent applications, sequences, GenBank citations, and other publications referred to in this application are herein incorporated by reference in their entirety.

TABLE 1
Candidate Gene Polymorphisms

| | Genotyped in | | |
|---|---|---|---|
| Polymorphism | Dataset | Alleles | HGBASE ID |
| 5HTT-01 | ANP2, B | A > C | SNP000007317 |
| 5HTT-06 | ANP2, P | Multiple 22 | IND001026444 |
| | | bp VNTR | |
| ADRB1-02 | ANP2 | G/C | SNP000003383 |
| ADRB2-01 | AN | C > T | SNP000008095 |
| ADRB2-02 | AN | C > T | SNP000008096 |
| ADRB2-03 | AN | A > G | SNP000003429 |
| ADRB2-04 | ANP1 | C > G | SNP000002717 |
| ADRB3-01 | AN, B | T > C | SNP000000522 |
| ADRB3-02 | AN, B | C > A | SNP000003415 |
| ADRB3-03 | ANP1 | C > G | SNP000006932 |
| ADRB3-06 | ANP1 | C > T | SNP001026445 |
| CART-02 | ANP1 | C > T | SNP001026480 |
| CCK-01 | ANP1, B | T > C | SNP000002386 |
| COMT-01 | ANP2, P | A > G | SNP000000140 |
| COMT-02 | ANP1 | C > G | SNP000003436 |
| COMT-03 | ANP1 | T > C | SNP000006653 |
| COMT-04 | ANP1 | C > G | SNP000006889 |
| COMT-06 | ANP1 | A > G | SNP000007209 |
| DAT-01 | ANP1 | 30 bp VNTR | No Seq. in HGVbase |
| | | (5 or 6) | |
| DAT-02 | ANP1 | G > A | SNP000223189 |
| DAT-12 | P | Multiple 40 | No Seq. in HGVbase |
| | | bp VNTR | |
| DBH-01 | ANP1 | T > C | SNP000002438 |
| DBH-09 | ANP1 | G > T | SNP000007898 |
| DRD1-03 | ANP2, B, R | G > A | SNP000002472 |
| DRD1-04 | ANP2, B, R | A > G | SNP000002473 |
| DRD1-05 | ANP2, B, R | C > T | SNP000003715 |
| DRD2-11 | ANP1, B | T > C | SNP000003288 |
| DRD2-23 | P | C > G | SNP000000181 |
| DRD2-24 | AN, B, P | T > C | SNP000000403 |
| DRD2-25 | AN, B, P | C > T | SNP000006629 |
| DRD2-35 | ANP1, B | G > T | SNP000064325 |
| DRD2-42 | ANP1, B | C > T | SNP000003286 |
| DRD2-43 | ANP2, P | —> C | IND000002594 |
| DRD3-01 | ANP2, P | A > G | SNP000000153 |
| DRD4-01 | ANP1, P | Multiple 48 | STR000063384 |
| | | bp VNTR | |
| ESR1-02 | ANP1, B | C > T | SNP000670004 |
| GLUL-02 | R | A > G | SNP000014629 |
| GOLF-01 | R | A > C | SNP000505465 |
| HCRTR1-01 | ANP1, S | C > T | SNP000779462 |
| HCRTR1-02 | ANP1, S | A > G | SNP001026446 |
| HCRTR1-03 | S | A > G | No Seq. in HGVbase |
| HCRTR1-04 | S | A > G | SNP001026447 |
| HCRTR1-05 | ANP1, S | A > G | SNP001026448 |
| HCRTR1-06 | S | A > G | SNP000777171 |
| HCRTR1-07 | ANP1, S | A > G | SNP001026449 |
| HCRTR1-08 | S | T > C | SNP001026450 |
| HCRTR1-09 | S | G > A | SNP001026451 |
| HCRTR1-11 | S | T > C | No Seq. in HGVbase |
| HCRTR1-12 | S | G > T | SNP001026452 |
| HCRTR1-13 | S | T > C | No Seq. in HGVbase |
| HCRTR2-03 | ANP1 | T > A | SNP001026481 |
| HCRTR2-04 | ANP1 | G > A | SNP001026482 |
| HSOBRGRP-01 | ANP1 | G > A | SNP000013585 |
| HSOBRGRP-03 | ANP1 | G > A | SNP000014761 |
| HTR1A-16 | ANP1, S | A > G | SNP001026453 |
| HTR1A-21 | ANP1, B, S | G > C | SNP000007100 |
| HTR1B-01 | ANP1, B | C > T | SNP000006652 |
| HTR1B-02 | ANP1, B | C > G | SNP000007238 |
| HTR1B-03 | ANP1, B | A > G | SNP000008028 |
| HTR1B-04 | ANP1, B | T > G | SNP001026454 |
| HTR1D-02 | ANP2, B, S | T > C | SNP000006432 |
| HTR1D-03 | ANP2, B, S | C > T | SNP000083091 |
| HTR1D-05 | ANP2, B, S | C > T | SNP000083015 |
| HTR1D-06 | ANP2, B, S | A > G | SNP000080270 |
| HTR2A-01 | ANP2, B, S | G > A | SNP000006269 |
| HTR2A-06 | P | C > T | SNP000000139 |
| HTR2A-10 | ANP2, B | T > C | SNP000006912 |
| HTR2A-18 | ANP1, B | A > G | SNP000007068 |
| HTR2C-01 | P | G/C | SNP000002388 |
| HTR2C-02 | ANP1, B | G > T | SNP000006414 |
| HTR5A-01 | ANP1 | T > A | SNP000006933 |
| HTR5A-03 | ANP1 | G > C | SNP000063136 |
| MC3R-01 | ANP1, S | C > A | SNP001026455 |
| MAOA-01 | ANP1 | T > C | SNP000005032 |
| MC3R-02 | ANP1, S | T > C | SNP001026456 |
| NPY-02 | S | C > T | SNP001026457 |
| NPY-03 | S | G > A | SNP001026458 |
| NPY-04 | ANP1, S | A > G | SNP001026459 |
| NPY-05 | S | G > T | SNP001026460 |
| NPY-06 | S | G > C | SNP001026461 |
| NPY-13 | S | G > C | SNP001026462 |
| NPY-17 | S | G > T | SNP001026465 |
| NPY-21 | S | A > C | SNP001026466 |
| NPY-22 | S | C > T | SNP000063703 |
| NPY-24 | S | C > T | SNP001026467 |
| NPY-25 | ANP1, S | G > A | SNP001026468 |
| NPY-26 | S | A > G | SNP001026471 |
| NPY-30 | S | A > G | SNP001026478 |
| NPY-37 | S | T > A | SNP000569013 |
| NPY-38 | ANP1, S | A > G | SNP001026463 |
| NPY-39 | S | A > G | SNP000008942 |
| NPY-42 | S | G > C | SNP001026469 |
| NPY-43 | S | T > C | SNP001026464 |
| NPY-44 | S | C > A | SNP001026472 |
| NPY-45 | S | A > G | SNP001026470 |
| OPRM1-01 | R, P | A > G | SNP000002579 |
| OPRD1-01 | ANP2, B | T > C | SNP001026473 |
| OPRD1-03 | ANP2, B | G > A | SNP000085600 |
| OPRD1-05 | ANP2, B | A > T | SNP000066640 |
| OPRD1-06 | ANP2, B | T > G | SNP000063484 |
| OPRD1-07 | ANP2, B, S | A > G | SNP000066643 |
| OPRD1-08 | ANP2, B | T > C | SNP000063485 |
| SVAT-01 | R | A > C | No Seq. in HGVbase |
| SVAT-02 | R | C > T | No Seq. in HGVbase |
| TH-01 | ANP1 | A > G | SNP000002595 |
| TPH-02 | P | C > A | SNP000003341 |
| TPH-03 | S | G > A | SNP001026474 |
| TPH-04 | S | T > G | SNP000387490 |
| TPH-05 | S | A > C | SNP000387447 |
| TPH-06 | S | A > G | SNP000387446 |
| TPH-07 | S | A > C | SNP001026475 |
| TPH-08 | S | C > T | SNP000379298 |
| TPH-09 | S | C > T | SNP000846284 |
| TPH-10 | S | C > T | SNP001026476 |
| TRH-04 | AN | C > G | SNP000006843 |
| TRH-05 | ANP1 | T > C | SNP000006103 |
| TRH-06 | ANP1 | A > G | SNP000007151 |
| TRHR-04 | ANP1 | T > C | SNP000006880 |
| TRHR-06 | ANP1 | C > G | SNP000007377 |

TABLE 2

HTR1D and OPRD1 Sequencing Primers

| | | | |
|---|---|---|---|
| Gene | Primer | Sequence¹ | Position² |

| | | | |
|---|---|---|---|
| HTR1D | PF-0076 | GGAGACTGAGGCAGGACAATCG | −1341 −> −1320 |
| | | (SEQ ID NO: 24) | |
| HTR1D | PR-0077 | GGTTTTCCCAGGTTCATCTTGAC | −598 −> −620 |
| | | (SEQ ID NO: 25) | |
| HTR1D | PF-0075 | CACATCACCCTCCCTGTATTC | −902 −> −494 |
| | | (SEQ ID NO: 26) | |
| HTR1D | PR-0074 | CAAGATGTCTCAGGGTCCTG | −291 −> −310 |
| | | (SEQ ID NO: 27) | |
| HTR1D | PF-0078 | GACTGCTTCTCTGAATCGGCTG | −577 −> −556 |
| | | (SEQ ID NO: 28) | |
| HTR1D | PR-0079 | TGATGACGGAAAGGACCACG | 145 −> −126 |
| | | (SEQ ID NO: 29) | |
| HTRID | PF-0010 | GACAACCTTGAAGGAAGGAG | −61 −> −42 |
| | | (SEQ ID NO: 30) | |
| HTR1D | PF-0080 | GGTTTCCATCTTGGTAATGC | 258 −> 277 |
| | | (SEQ ID NO: 31) | |
| HTR1D | PR-0011 | CCGATGAGGTTACAGGACAC | 1185 −> 1166 |
| | | (SEQ ID NO: 32) | |
| OPRD1 | PF-0079 | GCAGTGTCCCTTCCTCAGAGTTG | IVS1 − 1917 −> IVS1 − 1895 |
| | | (SEQ ID NO: 33) | |
| OPRD1 | PR-0080 | AAAGAAAAATCCTAAGCCAGGTGC | IVS1 − 1161 −> IVS1 − 1184 |
| | | (SEQ ID NO: 34) | |
| OPRD1 | PF-0081 | TCAAGCAATCCACCTGCCC | IVS1 − 1247 −> IVS1 − 1229 |
| | | (SEQ ID NO: 35) | |
| OPRD1 | PR-0082 | CCCGACAACAGAAGCAAAAGG | IVS1 − 473 −> IVS1 − 493 |
| | | (SEQ ID NO: 36) | |
| OPRD1 | PF-0083 | AGAGAGGGGGTTTCACCGTG | IVS1 − 661 −> IVS1 − 642 |
| | | (SEQ ID NO: 37) | |
| OPRD1 | PR-0084 | TGGCAGACAGCGATGTAGCG | 455 −> 436 |
| | | (SEQ ID NO: 38) | |
| OPRD1 | PF-0085 | GGTTTCCATCTTGGTAATGC | IVS1 − 114 −> IVS1 − 95 |
| | | (SEQ ID NO: 39) | |
| OPRD1 | PR-0086 | CATTGGTTGACCTTCTTCTACACTCC | IVS2 + 187 −> IVS2 + 162 |
| | | (SEQ ID NO: 40 | |
| OPRD1 | PF-0087 | GGAGTGTAGAAGAAGGTCAACCAATG | IVS2 + 162 −> IVS2 + 187 |
| | | (SEQ ID NO: 41) | |
| OPRD1 | PR-0088 | CCAGATGCCAGCAGTAGAAGATTC | IVS2 + 725 −> IVS2 + 702 |
| | | (SEQ ID NO: 42) | |
| OPRD1 | PF-0089 | ACCCAGCCTCCTGTTGATGG | IVS2 + 677 −> IVS2 + 696 |
| | | (SEQ ID NO: 43) | |
| OPRD1 | PR-0090 | CCTGACCTCTCTGATTCTGTTTCC | IVS2 + 1382 −> IVS2 + 1359 |
| | | (SEQ ID NO: 44) | |
| OPRD1 | PF-0091 | GGGACTCCTACCTCCATTTGACTG | IVS2 + 1332 −> IVS2 + 1355 |
| | | (SEQ ID NO: 45) | |
| OPRD1 | PR-0092 | GGGGTGTTGTGGGATTCTGATAC | IVS2 + 2003 −> IVS2 + 1981 |
| | | (SEQ ID NO: 46) | |

¹5′ to 3′.
²Position relative to the A of ATG initiation codon.

TABLE 3
HTR1D, OPRD1, and HCRTR1 SNPs Genotyped

| SNP¹ | Allele 1 | Allele 2 | SNP ID² | Coding region | Source | % A1³ |
|---|---|---|---|---|---|---|
| HTR1D-05 (−1123T > C) | T | C | SNP000083015 | No, 5′ of coding | This Study | 31.3 |
| HTR1D-03 (−628T > C) | T | C | SNP000083091 | No, 5′ of coding | This Study | 14.1 |
| HTR1D-02 (1080C > T) | C | T | SNP000006432 | Yes, silent | Reference 40 | 9.4 |
| HTR1D-06 (2190A > G) | A | G | SNP000080270 | No, 3′ of coding | This Study | 65.6 |
| OPRD1-06 (80T > G) | T | G | SNP000063484 | Yes, F27C | Reference 42 | |
| OPRD1-01 (8214T > C) | T | C | SNP001026473 | No, IVS 1 | TSC0110129 | |
| OPRD1-03 (23340G > A) | G | A | SNP000085600 | No, IVS 1 | TSC0110127 | |
| OPRD1-07 (47821A > G) | A | G | SNP000066643 | No, IVS 2 | This Study | 41.1 |
| OPRD1-05 (51502A > T) | A | T | SNP000066640 | No, 3′ of coding | TSC0110133 | |
| HCRTR1 (114C > T) | C | T | SNP000779462 | Yes, silent | This Study | 38.7 |
| HCRTR1 (846A > G) | A | G | SNP001026448 | No, IVS 2 | This Study | 59.4 |
| HCRTR1 (7757A > G) | A | G | SNP001026446 | Yes, Silent | This Study | 35.5 |
| HCRTR1 (8793C > T) | C | T | SNP001026450 | No, 3′ of coding | This Study | 57.8 |

¹From the ATG of the candidate gene genomic sequence.
²HGVBASE at http://hgvbase.cgb.ki.se.
³Percent allele 1 from 32 unrelated CEPH DNAs obtained by resequencing.

TABLE 4
Polymorphisms Genotyped at DRD2

| Polymorphism/Location* | Source | Frequency-CV† | Groups |
|---|---|---|---|
| DRD2-43 | Arinami et al., 1997; HGBASE: | 90/10 | Case: control; |
| −141 -> C*; promoter region | IND000002594; NCBI SNP ID: rs1799732 | | families |
| DRD2-11 | Kidd et al., 1998; HGBASE: SNP000003288; | 55/45 | Case: control |
| IVS2-2739T > C | NCBI SNP ID: rs1800498 | | |
| DRD2-23 | Itokawa et al., 1993; HGBASE: | NA | Families |
| 932C > G, exon 7, S311C | SNP000000181; NCBI SNP ID: rs1801028 | | |
| DRD2-24 | Sarkar et al., 1991; HGBASE: | 70/30 | Case: control; |
| 939T > C, exon 7, silent | SNP000000403; NCBI SNP ID: rs6275 | | families |
| DRD2-25 | Cargill et al., 1999; HGBASE: | 50/50 | Case: control; |
| 957C > T, exon 7, silent | SNP000006629; NCBI SNP ID: rs6277 | | families |
| DRD2-35 | Cargill et al., 1999; HGBASE: | 80/20 | Case: control |
| 14664G > T*, ˜5 kb 3′ of STP | SNP000007297; NCBI SNP ID: rs6278 | | |
| DRD2-42 | Hauge et al., 1991; HGBASE: | 76/24 | Case: control |
| 24490C > T*, ˜11 kb 3′ of STP | SNP000003286; NCBI SNP ID: rs1800497 | | |

*relative to the A of ATG initiation codon, mutation type indicated when appropriate.
†100 Coriell variation panel samples

TABLE 5

HTR1D, OPRD1, and HCRTR1 TaqMan Primers and Probes

| | Forward Primer | Allele 1 Probe |
|---|---|---|
| SNP | Reverse Primer | Allele 2 Probe |
| HTR1D | ATAAAACTGTACACAGGGAA | FAM-aaggccatcaggaaaAaaaccaaat-TAMRA |
| (−1123T > C) | (SEQ ID NO: 47) | (SEQ ID NO: 49) |
| | CTTTGTAGAGAAATACATTGTAAC | VIC-taaaggccatcaggaaaGaaaccaaat-TAMRA |
| | (SEQ ID NO: 48) | (SEQ ID NO: 50) |
| HTR1D | CGGTTTTCCCAGGTTC | FAM-tgacgcatcctAagctact-TAMRA |
| (−628T > C) | (SEQ ID NO: 51) | (SEQ ID NO: 53) |
| | TCAGTGGGATAGGAACC | TET-acgcatcctGagctactta-TAMRA |
| | (SEQ ID NO: 52) | (SEQ ID NO: 54) |
| HTR1D | GAAAGGGACAATTTTCTGAA | FAM-aaactcttcGttaaacacagtg-TAMRA¹ |
| (1080C < T) | (SEQ ID NO: 55) | (SEQ ID NO: 57) |
| | CCCTCATCAATCCAATAATC | TET-aactcttcAttaaacacagtgt-TAMRA¹ |
| | (SEQ ID NO: 56) | (SEQ ID NO: 58) |
| HTR1D | GTAGATTGACCGGCTTTA | FAM-cccacccAccgcaagc-MGB |
| (2190A > G) | (SEQ ID NO: 59) | (SEQ ID NO: 61) |
| | ATGGTGTCCCACTCAA | TET-cccacccGccgcaag-MGB |
| | (SEQ ID NO: 60) | (SEQ ID NO: 62) |
| OPRD1 | CCGCTCTTCGCCAA | FAM-cgcctTccccagcgct-TAMRA |
| (80T > G) | (SEQ ID NO: 63) | (SEQ ID NO: 65) |
| | ATTGCCAGGGCGAG | TET-cctagcgcctGcccca-TAMRA |
| | (SEQ ID NO 64) | (SEQ ID NO: 66) |
| OPRD1 | TGGCTCACACCTGTAA | FAM-cacctggggtcaAgagtttgag-TAMRA |
| (8214T > C) | (SEQ ID NO: 67) | (SEQ ID NO: 69) |
| | ACAAAGCGAGATCCCA | TET-acctggggtcaGgagtttga-TAMRA |
| | (SEQ ID NO: 68) | (SEQ ID NO: 70) |
| OPRD1 | TGCTCACCTCCTGTG | FAM-tgcggattcaAtgggttat-TAMRA¹ |
| (23340A > G) | (SEQ ID NO: 71) | (SEQ ID NO: 73) |
| | CCAGTCTCCCTCCTAAG | TET-tgcggattcaGtgggtt-TAMRA¹ |
| | (SEQ ID NO: 72) | (SEQ ID NO: 74) |
| OPRD1 | TTCCAGACCAGCCTG | FAM-cctatctttactaaaaAtacaaaaatta-MGB |
| (47821A > G) | (SEQ ID NO: 75) | (SEQ ID NO: 77) |
| | GACTACAGACGCCCA | VIC-ccctatctttactaaaaGtacaaaaatta-MGB |
| | (SEQ ID NO: 76) | (SEQ ID NO: 78) |
| OPRD1 | AGATTTGGTCAGCAGATAG | FAM-tgtggcctcaActttgg-TAMRA¹ |
| (51502A > T) | (SEQ ID NO: 79) | (SEQ ID NO: 81) |
| | TTGCCCCTTGCTAGAA | TET-tgtggcctcaTctttgg-TAMRA¹ |
| | (SEQ ID NO: 80) | (SEQ ID NO: 82) |
| HCRTR1 | GACCCACTCATACTGTTT | FAM-agataatcGcgccacagatagc-TAMRA |
| (114C > T) | (SEQ ID NO: 83) | (SEQ ID NO: 85) |
| | AGACTATGAAGATGAGTTTCT | VIC-agataatcAcgccacagatagcg-TAMRA |
| | (SEQ ID NO: 84) | (SEQ ID NO: 86) |
| HCRTR1 | GTGGAAACCAGGATGTC | FAM-tggggttagtggAgtggaagg-TAMRA |
| (846A > G) | (SEQ ID NO: 87) | (SEQ ID NO: 89) |
| | ATACAAACTGAGAGAAGCC | VIC-tggggttagtggGgtggaa-TAMRA |
| | (SEQ ID NO: 88) | (SEQ ID NO: 90) |
| HCRTR1 | GCCACAAGTCCTTGTC | FAM agccgatgctccAtctcca-TAMRA |
| (7757A > G) | (SEQ ID NO: 91) | (SEQ ID NO: 93) |
| | TGAGCACCACATGCT | VIC ccgatgctccGtctccaaaatc-TAMRA |
| | (SEQ ID NO: 92) | (SEQ ID NO: 94) |
| HCRTR1 | CTCTTTTTATCCTGTGAGTTC | FAM-agaaaataggcAcaagccttggt-TAMRA |
| (8793C > T) | (SEQ ID NO: 95) | (SEQ ID NO: 97) |
| | TACTGTTATCTTCATCTTCTTG | TET-aataggcGcaagccttggtt-TAMRA |
| | (SEQ ID NO: 96) | (SEQ ID NO: 98) |

¹Synthesized with propyne-T.

TABLE 6
DRD2 Case: Control Contingency Analyses. Numbers shown are χ² (p).

| | AN | | AN1 | | AN2 | |
|---|---|---|---|---|---|---|
| | Genotypes | Alleles | Genotypes | Alleles | Genotypes | Alleles |
| DRD2-43 | 5.201 | 4.769 | 4.272 | 3.883 | 2.928 | 2.646 |
| (IND000002594) | (0.023) | (0.029) | (0.039) | (0.049) | (0.087) | (0.104) |
| DRD2-11 | 1.754 | 0.244 | 4.525 | 2.105 | 0.538 | 0.423 |
| (SNP000003288) | (0.416) | (0.662) | (0.104) | (0.147) | (0.764) | (0.516) |
| DRD2-24 | 0.898 | 0.060 | 0.902 | 0.000 | 0.594 | 0.182 |
| (SNP000000403) | (0.638) | (0.807) | (0.637) | (0.992) | (0.743) | (0.67) |
| DRD2-25 | 0.505 | 0.080 | 1.406 | 1.046 | 0.506 | 0.333 |
| (SNP000006629) | (0.777) | (0.777) | (0.495) | (0.306) | (0.776) | (0.564) |
| DRD2-35 | 4.443 | 0.966 | 2.870 | 2.415 | 5.905 | 0.005 |
| (SNP000007297) | (0.108) | (0.326) | (0.238) | (0.120) | (0.052) | (0.941) |
| DRD2-42 | 3.438 | 1.156 | 2.423 | 1.450 | 6.683 | 0.334 |
| (SNP000003286) | (0.179) | (0.282) | (0.298) | (0.229) | (0.035) | (0.564) |

TABLE 7
Pairwise LD Among DRD2 SNPs - AN Probands and EAF Samples

| | DRD2-43 | DRD2-11 | DRD2-24 | DRD2-25 | DRD2-35 | DRD2-42 |
|---|---|---|---|---|---|---|
| DRD2-43 | | 0.104 | 1.130 | 0.002 | 3.045 | 0.417 |
| | | (0.747) | (0.288) | (0.966) | (0.081) | (0.520) |
| DRD2-11 | 6.913 | | 24.845 | 105.036 | 39.916 | 9.757 |
| | (0.009) | | (<10−5) | (<10⁻⁵) | (<10⁻⁵) | (0.002) |
| DRD2-24 | 10.360 | 44.883 | | 58.644 | 0.660 | 8.365 |
| | (0.001) | (<10⁻⁵) | | (<10⁻⁵) | (0.417) | (0.004) |
| DRD2-25 | 7.974 | 169.712 | 100.497 | | 28.221 | 8.305 |
| | (0.005) | (<10⁻⁵) | (<10⁻⁵) | | (<10⁻⁵) | (0.004) |
| DRD2-35 | 0.044 | 51.955 | 9.212 | 42.984 | | 59.352 |
| | (0.834) | (<10⁻⁵) | (0.002) | (<10⁻⁵) | | (<10⁻⁵) |
| DRD2-42 | 0.037 | 22.666 | 13.160 | 19.595 | 104.497 | |
| | (0.847) | (<10⁻⁵) | (0.003) | (10⁻⁴) | (<10⁻⁵) | |

Numbers shown are χ² (p). Top right = EAF; bottom left = AN probands. Grey background indicates significant LD (p < 0.05).

TABLE 8
HTR1D and OPRD1 SNP genotype and allele frequencies in AN and EAF samples

| | | | Genotypes | | | | | | Alleles | | | |
|---|---|---|---|---|---|---|---|---|---|---|---|---|
| SNP | Sample | N | N₁₁ | P₁₁ | N₁₂ | P₁₂ | N₂₂ | P₂₂ | N₁ | P₁ | N₂ | P₂ |
| HTR1D(−1123T > C) | AN | 181 | 11 | 0.06 | 74 | 0.41 | 96 | 0.53 | 96 | 0.27 | 266 | 0.74 |
| | EAF | 91 | 9 | 0.10 | 42 | 0.46 | 40 | 0.44 | 60 | 0.33 | 122 | 0.67 |
| HTR1D(−628T > C) | AN | 188 | 5 | 0.03 | 40 | 0.21 | 143 | 0.76 | 50 | 0.13 | 326 | 0.87 |
| | EAF | 85 | 1 | 0.01 | 28 | 0.33 | 56 | 0.66 | 30 | 0.18 | 140 | 0.82 |
| HTR1D(1080C > T) | AN | 182 | 2 | 0.01 | 37 | 0.2 | 143 | 0.79 | 41 | 0.11 | 323 | 0.89 |
| | EAF | 87 | 1 | 0.01 | 6 | 0.07 | 80 | 0.92 | 8 | 0.05 | 166 | 0.95 |
| HTR1D(2190A > G) | AN | 182 | 92 | 0.51 | 73 | 0.40 | 17 | 0.09 | 257 | 0.71 | 107 | 0.29 |
| | EAF | 91 | 36 | 0.40 | 44 | 0.48 | 11 | 0.12 | 116 | 0.64 | 66 | 0.36 |
| OPRD1(80T > G) | AN | 172 | 140 | 0.81 | 27 | 0.16 | 5 | 0.03 | 307 | 0.89 | 37 | 0.11 |
| | EAF | 90 | 71 | 0.79 | 19 | 0.21 | 0 | 0.00 | 161 | 0.89 | 19 | 0.11 |
| OPRD1(8214T > C) | AN | 181 | 61 | 0.34 | 82 | 0.45 | 38 | 0.21 | 204 | 0.56 | 158 | 0.44 |
| | EAF | 80 | 18 | 0.23 | 39 | 0.49 | 23 | 0.29 | 75 | 0.47 | 85 | 0.53 |
| OPRD1(23340A > G) | AN | 181 | 21 | 0.12 | 71 | 0.39 | 89 | 0.49 | 113 | 0.31 | 249 | 0.69 |
| | EAF | 89 | 15 | 0.17 | 41 | 0.46 | 33 | 0.37 | 71 | 0.40 | 107 | 0.60 |
| OPRD1(47821A > G) | AN | 176 | 58 | 0.33 | 88 | 0.50 | 30 | 0.17 | 204 | 0.58 | 148 | 0.42 |
| | EAF | 82 | 41 | 0.50 | 32 | 0.39 | 9 | 0.11 | 114 | 0.70 | 50 | 0.31 |
| OPRD1(51502A > T) | AN | 181 | 62 | 0.34 | 89 | 0.49 | 30 | 0.17 | 213 | 0.59 | 149 | 0.41 |
| | EAF | 87 | 41 | 0.47 | 35 | 0.40 | 11 | 0.13 | 117 | 0.67 | 57 | 0.33 |
| HCRTR1(114C > T) | AN | 174 | 40 | 0.23 | 74 | 0.43 | 60 | 0.34 | 154 | 0.44 | 194 | 0.56 |
| | EAF | 87 | 18 | 0.21 | 36 | 0.41 | 33 | 0.38 | 72 | 0.41 | 102 | 0.59 |
| HCRTR1(846A > G) | AN | 175 | 61 | 0.35 | 71 | 0.41 | 43 | 0.25 | 193 | 0.55 | 157 | 0.45 |
| | EAF | 86 | 36 | 0.42 | 35 | 0.41 | 16 | 0.19 | 107 | 0.62 | 67 | 0.39 |
| HCRTR1(7757A > G) | AN | 183 | 33 | 0.18 | 76 | 0.42 | 74 | 0.40 | 142 | 0.39 | 224 | 0.61 |
| | EAF | 82 | 11 | 0.13 | 36 | 0.44 | 35 | 0.43 | 58 | 0.35 | 106 | 0.65 |
| HCRTR1(8793C > T) | AN | 159 | 52 | 0.33 | 66 | 0.42 | 41 | 0.26 | 170 | 0.53 | 148 | 0.47 |
| | EAF | 98 | 37 | 0.38 | 44 | 0.45 | 17 | 0.17 | 118 | 0.60 | 78 | 0.40 |

N = sample size;
N_{11,12,22} and P_{11,12,22} = N and % for genotype 11, 12, 22, respectively.

TABLE 9
HTR1D and OPRD1 LD and Distance Between SNP Pairs in the AN Proband Sample¹

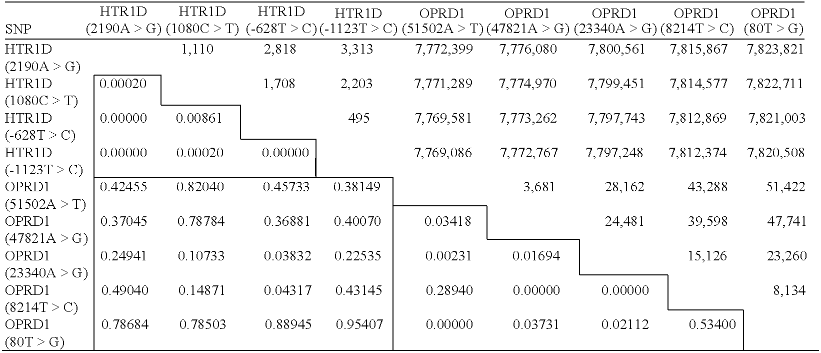

SNPs are presented in the distal to proximal orientation on chr1p in this table, p values derived empirically from likelihood ratio analyses are presented below diagonal and the inter-SNP distance in bp is presented above diagonal. Intragenic and intergenic likelihood ratio p values are outlined for clarity.

TABLE 10
Numbers and Percentages for DRD2 SNPs for Genotypes and Alleles^{a}

| | | | Genotypes | | | Alleles | |
|---|---|---|---|---|---|---|---|
| SNP (alleles) | Sample | N | N₁₁ (%) | N₁₂ (%) | N₂₂ (%) | N₁ (%) | N₂ (%) |
| DRD2-43 | AN | 178 | 0 (0) | 21 (12) | 157 (88) | 21 (6) | 335 (94) |
| (IND000002594) | | | | | | | |
| (1 = —, 2 = C) | EAF | 89 | 0 (0) | 20 (22) | 69 (78) | 20 (11) | 158 (89) |
| DRD2-11 | AN | 114 | 51 (45) | 43 (38) | 20 (18) | 145 (64) | 83 (36) |
| (SNP000003288) | | | | | | | |
| (1 = T, 2 = C) | EAF | 85 | 32 (38) | 40 (47) | 13 (15) | 104 (61) | 66 (39) |
| DRD2-24 | AN | 183 | 99 (54) | 77 (42) | 7 (4) | 275 (75) | 91 (25) |
| (SNP000000181) | | | | | | | |
| (1 = T, 2 = C) | EAF | 92 | 53 (58) | 34 (37) | 5 (5) | 140 (76) | 44 (24) |
| DRD2-25 | AN | 183 | 69 (38) | 82 (45) | 32 (17) | 220 (60) | 146 (40) |
| (SNP000006629) | | | | | | | |
| (1 = C, 2 = T) | EAF | 85 | 29 (34) | 42 (49) | 14 (17) | 100 (59) | 70 (41) |
| DRD2-35 | AN | 114 | 86 (75) | 24 (21) | 4 (4) | 196 (86) | 32 (14) |
| (SNP000007297) | | | | | | | |
| (1 = G, 2 = T) | EAF | 88 | 58 (66) | 29 (33) | 1 (1) | 145 (82) | 31 (18) |
| DRD2-42 | AN | 122 | 5 (4) | 36 (30) | 81 (66) | 46 (19) | 198 (81) |
| (SNP000003286) | | | | | | | |
| (1 = T, 2 = C) | EAF | 84 | 2 (2) | 35 (42) | 47 (56) | 39 (23) | 129 (77) |

^{a}AN: Anorexia Nervosa Sample;
EAF: European-American Female Control Sample;
N, total sample size;
N₁₁ (%): number and percentage of observed genotype 11;
N₁₂ (%): number and percentage of observed genotype 12;
N₂₂ (%): number and percentage of observed genotype 22.

TABLE 11
DRD2 SNP TDT Results Probands and Parents Only

| | Transmitted/ | | | | |
|---|---|---|---|---|---|
| SNP | nontransmitted | Mean | Variance | Z | p |
| DRD2-43 | 13/2 | 7.500 | 3.750 | 2.582 | 0.010 |
| DRD2-24 | 30/17 | 23.500 | 11.750 | 1.750 | 0.080 |
| DRD2-25 | 39/18 | 28.500 | 14.250 | 2.649 | 0.008 |

Z = normalized z score of transmission statistic with p = two sided p value

TABLE 12
TDT Results for AN-ARP Probands

| | Allele | b | c | Chi-Sq | W | Mean(A) | Var(V) | z′ | p |
|---|---|---|---|---|---|---|---|---|---|
| 5htt-01 | 1 | 21 | 12 | 2.455 | 21 | 16.5 | 8.25 | 1.393 | 0.164 |
| 5HTT-06 | 2 | 19 | 34 | 4.245 | 19 | 27 | 13.5 | 2.041 | 0.041 |
| adrb2-01 | 1 | 33 | 18 | 4.412 | 33 | 25.5 | 12.75 | 1.960 | 0.050 |
| adrb2-02 | 1 | 28 | 19 | 1.723 | 28 | 23.5 | 11.75 | 1.167 | 0.243 |
| adrb203rg | 1 | 17 | 11 | 1.286 | 17 | 14.5 | 7.25 | 0.743 | 0.458 |
| adrb3-01 | 1 | 7 | 11 | 0.889 | 7 | 9 | 4.5 | 0.707 | 0.480 |
| adrb3-02 | 1 | 5 | 11 | 2.25 | 5 | 8 | 4 | 1.250 | 0.211 |
| COMT-01 | 2 | 31 | 38 | 0.71 | 31 | 34.5 | 17.25 | 0.722 | 0.470 |
| DAT-12 | 2 | 17 | 22 | 0.641 | 17 | 19.5 | 9.75 | 0.641 | 0.522 |
| | 3 | 21 | 18 | 0.231 | 21 | 19.5 | 9.75 | 0.320 | 0.749 |
| | 1 | 1 | 0 | 1 | 1 | 0.5 | 0.25 | 0.000 | 1.000 |
| | 4 | 1 | 0 | 1 | 1 | 0.5 | 0.25 | 0.000 | 1.000 |
| drd1-03rg | 1 | 17 | 12 | 0.862 | 17 | 14.5 | 7.25 | 0.743 | 0.458 |
| | 2 | 12 | 17 | 0.862 | 12 | 14.5 | 7.25 | 0.743 | 0.458 |
| drd1-04rg | 2 | 21 | 25 | 0.348 | 21 | 23 | 11.5 | 0.442 | 0.659 |
| | 1 | 25 | 21 | 0.348 | 25 | 23 | 11.5 | 0.442 | 0.659 |
| drd1-05rg | 2 | 15 | 22 | 1.324 | 15 | 18.5 | 9.25 | 0.986 | 0.324 |
| DRD2-23 | 1 | 3 | 3 | 0 | 3 | 3 | 1.5 | −0.408 | 0.683 |
| drd2-24 | 1 | 30 | 18 | 3 | 30 | 24 | 12 | 1.588 | 0.112 |
| drd2-25 | 1 | 40 | 19 | 7.475 | 40 | 29.5 | 14.75 | 2.604 | 0.009 |
| DRD2-43 | 2 | 13 | 2 | 8.067 | 13 | 7.5 | 3.75 | 2.582 | 0.010 |
| DRD3-01 | 2 | 35 | 16 | 7.078 | 37 | 27 | 13 | 2.635 | 0.008 |
| DRD4-01 | 6 | 14 | 14 | 0 | 14 | 14 | 7 | −0.189 | 0.850 |
| | 1 | 13 | 5 | 3.556 | 13 | 9 | 4.5 | 1.650 | 0.099 |
| | 3 | 22 | 25 | 0.191 | 22 | 23.5 | 11.75 | 0.292 | 0.770 |
| | 2 | 6 | 9 | 0.6 | 6 | 7.5 | 3.75 | 0.516 | 0.606 |
| | 4 | 0 | 1 | 1 | 0 | 0.5 | 0.25 | 0.000 | 1.000 |
| | 7 | 0 | 1 | 1 | 0 | 0.5 | 0.25 | 0.000 | 1.000 |
| glul-02rg | 1 | 20 | 19 | 0.026 | 20 | 19.5 | 9.75 | 0.000 | 1.000 |
| golf-01rg | 2 | 10 | 3 | 3.769 | 10 | 6.5 | 3.25 | 1.664 | 0.096 |
| htr1d-03 | 1 | 7 | 18 | 4.84 | 7 | 12.5 | 6.25 | 2.000 | 0.046 |
| htr1d-05 | 1 | 13 | 26 | 4.333 | 13 | 19.5 | 9.75 | 1.922 | 0.055 |
| htr1d-06 | 1 | 24 | 13 | 3.27 | 24 | 18.5 | 9.25 | 1.644 | 0.100 |
| htr2a-01 | 1 | 29 | 30 | 0.017 | 29 | 29.5 | 14.75 | 0.000 | 1.000 |
| HTR2A-06 | 2 | 29 | 33 | 0.258 | 29 | 31 | 15.5 | 0.381 | 0.703 |
| HTR2A-10 | 1 | 7 | 7 | 0 | 7 | 7 | 3.5 | −0.267 | 0.790 |
| oprd1-01 | 2 | 25 | 27 | 0.077 | 25 | 26 | 13 | 0.139 | 0.890 |
| oprd1-03 | 2 | 27 | 23 | 0.32 | 27 | 25 | 12.5 | 0.424 | 0.672 |
| oprd1-05 | 1 | 22 | 36 | 3.379 | 22 | 29.5 | 14.75 | 1.823 | 0.068 |
| oprd1-06 | 1 | 14 | 9 | 1.087 | 14 | 11.5 | 5.75 | 0.834 | 0.404 |
| oprd1-07 | 1 | 23 | 38 | 3.689 | 23 | 31 | 15.5 | 1.905 | 0.057 |
| | 2 | 38 | 23 | 3.689 | 38 | 31 | 15.5 | 1.651 | 0.099 |
| oprd1-08 | 1 | 18 | 27 | 1.8 | 18 | 23 | 11.5 | 1.327 | 0.185 |
| oprm1-01rg | 1 | 12 | 6 | 2 | 12 | 9 | 4.5 | 1.179 | 0.238 |
| OPRM1-01 | 2 | 8 | 12 | 0.8 | 8 | 10 | 5 | 0.671 | 0.502 |
| TPH-02 | 2 | 27 | 30 | 0.158 | 27 | 28.5 | 14.25 | 0.265 | 0.791 |
| svat-01rg | 2 | 1 | 0 | 1 | 1 | 0.5 | 0.25 | 0.000 | 1.000 |
| svat-02rg | 1 | 21 | 25 | 0.348 | 21 | 23 | 11.5 | 0.442 | 0.659 |
| trh-04 | 1 | 6 | 16 | 4.545 | 6 | 11 | 5.5 | 1.919 | 0.055 |

TABLE 13
Results of case: control association analyses, AN versus EAF

| SNP | N | χ² | p | OR | (95% CI) |
|---|---|---|---|---|---|
| HTR1D(−1123T > C) | | | | | |
| Genotypes | 272 | 2.59 | .27 | | |
| Alleles | 544 | 2.46 | .12 | .73 | .50-1.08 |
| HTR1D(−628T > C) | | | | | |
| Genotypes | 273 | 4.62 | .10* | | |
| Alleles | 546 | 1.77 | .18 | .72 | .44-1.17 |
| HTR1D(1080C > T) | | | | | |
| Genotypes | 269 | 7.92 | .01* | | |
| Alleles | 538 | 6.32 | .01 | 2.63 | 1.21-5.75 |
| HTR1D(2190A > G) | | | | | |
| Genotypes | 273 | 2.97 | .23 | | |
| Alleles | 546 | 2.64 | .10 | 1.37 | .94-1.99 |
| OPRD1(80T > G) | | | | | |
| Genotypes | 262 | 3.65* | .17 | | |
| Alleles | 524 | .01 | .94 | .98 | .55-1.76 |
| OPRD1(8214T > C) | | | | | |
| Genotypes | 261 | 3.87 | 0.14 | | |
| Alleles | 522 | 4.01 | 0.045 | 1.46 | 1.01-2.13 |
| OPRD1(23340A > G) | | | | | |
| Genotypes | 270 | 3.84 | 0.15 | | |
| Alleles | 540 | 4.00 | 0.046 | .68 | .47-.99 |
| OPRD1(47821A > G) | | | | | |
| Genotypes | 258 | 7.05 | 0.03 | | |
| Alleles | 516 | 6.32 | 0.01 | 0.61 | .41-.90 |
| OPRD1(51502A > T) | | | | | |
| Genotypes | 268 | 4.14 | 0.13 | | |
| Alleles | 536 | 3.51 | 0.06 | .70 | 0.48-1.02 |

*Indicates that the genotypic chi-square analysis required the use of Fisher's exact test due to low cell counts.

TABLE 14
OPRD1 and HTR1D haplotype frequency heterogeneity
analyses, AN vs. EAF

| | | |
|---|---|---|
| SNP Haplotype | χ2 | p* |

HTR1D

| | | |
|---|---|---|
| (−628T > C)/(1080C > T) | 8.90 | 0.01 |
| (−1123T > C)/(1080C > T) | 6.26 | 0.04 |
| (1080C > T)/(2190A > G) | 7.14 | 0.03 |
| (−1123T > C)/(−628T > C) | 3.34 | 0.19 |
| (−628T > C)/(2190A > G) | 3.28 | 0.22 |
| (−1123T > C)/(2190A > G) | 2.38 | 0.31 |
| (−628T > C)/(−1123T > C)/ | 7.52 | 0.16 |
| (1080C > T)/(2190A > G) | | |

OPRD1

| | | |
|---|---|---|
| (8214T > C)/(23340A > G) | 4.24 | 0.12 |
| (8214T > C)/(51502A > T) | 6.04 | 0.13 |
| (80T > G)/(8214T > C) | 5.00 | 0.22 |
| (8214T > C)/(47821A > G) | 8.82 | 0.04 |
| (23340A > G)/(51502A > T) | 5.54 | 0.16 |
| (80T > G)/(23340A > G) | 3.88 | 0.32 |
| (23340A > G)/(47821A > G) | 7.94 | 0.06 |
| (80T > G)/(51502A > T) | 2.86 | 0.43 |
| (47821A > G)/(51502A > T) | 8.90 | 0.06 |
| (80A > G)/(47821T > G) | 5.34 | 0.16 |
| (80A > G)/(8214T > C)/(23340A > G)/ | 25.14 | 0.05 |
| (47821T > G)/(51502A > T) | | |

*Based on 10,000 permutations.

TABLE 15
HTR1D and OPRD1 SNP TDT Results

| | | # informative | | |
|---|---|---|---|---|
| SNP | Freq (Allele1) | families | Z | p |
| HTR1D(−1123T > C) | 0.281 | 32 | −2.34 | 0.02 |
| HTR1D(−628T > C) | 0.150 | 23 | −2.50 | 0.01 |
| HTR1D(1080C > T) | 0.112 | 22 | 1.00 | 0.32 |
| HTR1D(2190A > G) | 0.701 | 34 | 2.03 | 0.04 |
| OPRD1(80T > G) | 0.887 | 22 | 0.82 | 0.41 |
| OPRD1(8214T > C) | 0.558 | 39 | 0.14 | 0.89 |
| OPRD1(23340A > G) | 0.327 | 40 | −0.57 | 0.57 |
| OPRD1(47821A > G) | 0.608 | 46 | −1.86 | 0.06 |
| OPRD1(51502A > T) | 0.615 | 45 | −1.89 | 0.06 |

Z = normalized z score of transmission statistic with p = two sided p value

TABLE 16
Contingency Table Analyses Performed for BN Probands.

| | | | | Type of | Contingency | |
|---|---|---|---|---|---|---|
| SNP | Alleles | HGBASE ID | Position | polymorphism | Test | BN vs. EAF |
| 5HTT-01 | A > C | SNP000007317 | −922 | 5′UTR | Allelic | 2.487 (0.228) |
| | | | | | Genotypic | 3.516 (0.172) |
| ADRB3-01 | T > C | SNP000000522 | 190 | W64R - Associated | A | 9.422 (0.009) |
| | | | | with | | |
| | | | | Hyperinsulinaemia | | |
| | | | | | G | 9.282 (0.010) |
| ADRB3-02 | C > A | SNP000003415 | IVS1+13 | splice site | A | 3.454 (0.178) |
| | | | | | G | 3.349 (0.187) |
| CCK-01 | T > C | SNP000002386 | −1173 | mutation in a GC | A | 5.201 (0.074) |
| | | | | box, a binding site | | |
| | | | | for | | |
| | | | | transcription factor | G | 5.438 (0.066) |
| | | | | Sp1 in the promoter | | |
| DRD1-03 | G > A | SNP000002472 | −94 | 5′ | A | 0.430 (0.806) |
| | | | | | G | 1.955 (0.376) |
| DRD1-04 | A > G | SNP000002473 | −48 | 5′UTR | A | 4.540 (0.103) |
| | | | | | G | 4.679 (0.096) |
| DRD1-05 | C > T | SNP000003715 | 1403 | 3′ UTR | A | 0.000 (1.000) |
| | | | | | G | 0.005 (0.997) |
| DRD2-11 | T > C | SNP000003288 | IVS2−2739 | | A | 0.140 (0.932) |
| | | | | | G | 0.296 (0.863) |
| DRD2-24 | T > C | SNP000000403 | 939 | silent | A | 5.009 (0.082) |
| | | | | | G | 4.976 (0.083) |
| DRD2-25 | C > T | SNP000006629 | 957 | silent | A | 1.848 (0.397) |
| | | | | | G | 1.883 (0.390) |
| DRD2-35 | G > T | SNP000064325 | 14664 | ˜728 bp 3′ of stop, 3′ | A | 1.110 (0.574) |
| | | | | UTR | | |
| | | | | | G | 1.537 (0.464) |
| DRD2-42 | C > T | SNP000003286 | 24490 | | A | 1.344 (0.511) |
| | | | | | G | 2.447 (0.294) |
| ESR1-02 | C > T | SNP000670004 | | intron | A | 9.366 (0.009) |
| | | | | | G | 11.179 (0.004) |
| HTR1A-21 | G > C | SNP000007100 | −1019 | 5′ | A | 2.921 (0.232) |
| | | | | | G | 3.325 (0.190) |
| HTR1B-01 | C > T | SNP000006652 | 129 | silent | A | 8.981 (0.011) |
| | | | | | G | 8.675 (0.013) |
| HTR1B-02 | C > G | SNP000007238 | 861 | silent | A | 7.414 (0.025) |
| | | | | | G | 7.493 (0.024) |
| HTR1B-03 | A > G | SNP000008028 | 1180 | 3′UTR, 7 bp from | A | 0.651 (0.722) |
| | | | | STP | | |
| | | | | | G | 1.358 (0.507) |
| HTR1B-04 | T > G | SNP001026454 | −261 | 5′ | A | 0.020 (0.990) |
| | | | | | G | 0.027 (0.987) |
| HTR1D-02 | T > C | SNP000006432 | 1080 | silent | A | 4.591 (0.101) |
| | | | | | G | 7.99 (0.018) |
| HTR1D-03 | C > T | SNP000083091 | −628 | 5′ | A | 10.535 (0.005) |
| | | | | | G | 13.084 (0.001) |
| HTR1D-05 | C > T | SNP000083015 | −1123 | 5′ | A | 0.127 (0.939) |
| | | | | | G | 1.643 (0.440) |
| HTR1D-06 | T > C | SNP000080270 | 2190 | 3′ | A | 0.198 (0.906) |
| | | | | | G | 0.944 (0.624) |
| HTR2A-01 | G > A | SNP000006269 | −1438 | 5′UTR | A | 0.005 (0.998) |
| | | | | | G | 5.613 (0.060)† |
| HTR2A-10 | T > C | SNP000006912 | 1354 | Y452H | A | 0.057 (0.972) |
| | | | | | G | 0.062 (0.970) |
| HTR2A-18 | A > G | SNP000007068 | −789 | 5′ | A | 0.021 (0.990) |
| | | | | | G | 0.535 (0.765) |
| HTR2C-02 | G > T | SNP000006414 | 2166 | 3′UTR - Located on | A | 0.246 (0.884) |
| | | | | X chromosome | | |
| OPRD1-01 | T > C | SNP001026473 | IVS1+8214 | | A | 2.669 (0.263) |
| | | | | | G | 2.632 (0.268) |
| OPRD1-03 | G > A | SNP000085600 | IVS1+23340 | | A | 2.634 (0.268) |
| | | | | | G | 2.535 (0.282) |
| OPRD1-05 | A > T | SNP000066640 | 51502 | 3′UTR | A | 1.178 (0.555) |
| | | | | | G | 2.285 (0.319) |
| OPRD1-06 | T > G | SNP000063484 | 80 | F27C | A | 0.664 (0.717) |
| | | | | | G | 1.591 (0.451) |
| OPRD1-07 | A > G | SNP000066643 | IVS2+898 | | A | 0.913 (0.634) |
| | | | | | G | 1.114 (0.573) |
| OPED1-08 | T > C | SNP000063485 | 921 | silent | A | 0.095 (0.954) |
| | | | | | G | 0.102 (0.951) |

*G = genotypes;
A = alleles.
Numbers shown are X² (p).

TABLE 17
TDT Analysis Results of BN-ARP Dataset

| SNP | test | p | SE* |
|---|---|---|---|
| 5HTT-01 | Permutation McNemar (MCMC) | 1.000 | 0.000 |
| ADRB3-01 | Permutation McNemar (MCMC) | 0.335 | −0.004 |
| ADRB3-02 | Permutation McNemar (MCMC) | 0.626 | −0.003 |
| CCK-01 | Permutation McNemar (MCMC) | 0.098 | −0.002 |
| DRD1-03 | Permutation McNemar (MCMC) | 0.893 | 0.000 |
| DRD1-04 | Permutation McNemar (MCMC) | 0.916 | 0.000 |
| DRD1-05 | Permutation McNemar (MCMC) | 0.837 | −0.002 |
| DRD2-11 | Permutation McNemar (MCMC) | 0.019 | −0.001 |
| | Asymptotic McNemar (X\hat{ }2) | 0.015 | |
| | Asymptotic Marginal (X\hat{ }2) | 0.015 | |
| | Permutation Marginal (MCMC) | 0.020 | −0.001 |
| DRD2-24 | Permutation McNemar (MCMC) | 0.009 | 0.000 |
| | Asymptotic McNemar (X\hat{ }2) | 0.007 | |
| | Asymptotic Marginal (X\hat{ }2) | 0.007 | |
| | Permutation Marginal (MCMC) | 0.010 | 0.000 |
| DRD2-25 | Permutation McNemar (MCMC) | 0.103 | −0.004 |
| | Asymptotic McNemar (X\hat{ }2) | 0.084 | |
| | Asymptotic Marginal (X\hat{ }2) | 0.084 | |
| | Permutation Marginal (MCMC) | 0.108 | −0.004 |
| DRD2-35 | Permutation McNemar (MCMC) | 0.312 | −0.005 |
| DRD2-42 | Permutation McNemar (MCMC) | 0.625 | −0.005 |
| ESR1-02 | Permutation McNemar (MCMC) | 0.745 | −0.003 |
| HTR1A-21 | Permutation McNemar (MCMC) | 0.766 | −0.004 |
| HTR1B-01 | Permutation McNemar (MCMC) | 0.209 | −0.006 |
| HTR1B-02 | Permutation McNemar (MCMC) | 0.315 | −0.008 |
| HTR1B-03 | Permutation McNemar (MCMC) | 0.061 | −0.001 |
| | Asymptotic McNemar (X\hat{ }2) | 0.048 | |
| | Asymptotic Marginal (X\hat{ }2) | 0.048 | |
| | Permutation Marginal (MCMC) | 0.066 | −0.002 |
| HTR1B-04 | Permutation McNemar (MCMC) | 0.084 | −0.004 |
| | Asymptotic McNemar (X\hat{ }2) | 0.066 | |
| | Asymptotic Marginal (X\hat{ }2) | 0.066 | |
| | Permutation Marginal (MCMC) | 0.079 | −0.003 |
| HTR1D-02 | Permutation McNemar (MCMC) | 0.535 | −0.004 |
| HTR1D-03 | Permutation McNemar (MCMC) | 0.510 | −0.005 |
| HTR1D-05 | Permutation McNemar (MCMC) | 0.584 | −0.006 |
| HTR1D-06 | Permutation McNemar (MCMC) | 0.346 | −0.011 |
| HTR2A-01 | Permutation McNemar (MCMC) | 0.676 | −0.005 |
| HTR2A-10 | Permutation McNemar (MCMC) | 0.594 | −0.004 |
| HTR2A-18 | Permutation McNemar (MCMC) | 0.007 | 0.000 |
| | Asymptotic McNemar (X\hat{ }2) | 0.005 | |
| | Asymptotic Marginal (X\hat{ }2) | 0.005 | |
| | Permutation Marginal (MCMC) | 0.007 | 0.000 |
| OPRD1-01 | Permutation McNemar (MCMC) | 1.000 | 0.000 |
| OPRD1-03 | Permutation McNemar (MCMC) | 0.457 | −0.008 |
| OPRD1-05 | Permutation McNemar (MCMC) | 0.153 | −0.006 |
| OPRD1-06 | Permutation McNemar (MCMC) | 0.668 | −0.003 |
| OPRD1-07 | Permutation McNemar (MCMC) | 0.597 | −0.007 |
| OPRD1-08 | Permutation McNemar (MCMC) | 1.000 | 0.000 |

TABLE 18
TDT Analysis Results in BN Probands and Parents Only

| SNP | A1 freq | # inf fam | S | E(S) | Var(S) | Z | P |
|---|---|---|---|---|---|---|---|
| 5HTT-01 | 0.217 | 22 | 15 | 15.5 | 7.25 | −0.186 | 0.853 |
| ADRB3-01 | 0.928 | 19 | 28 | 26.5 | 5.75 | 0.626 | 0.532 |
| ADRB3-02 | 0.927 | 17 | 26 | 24 | 5 | 0.894 | 0.371 |
| CCK-01 | 0.889 | 21 | 32 | 29 | 6 | 1.225 | 0.221 |
| DRD1-03 | 0.129 | 25 | 16 | 16.5 | 8.25 | −0.174 | 0.862 |
| DRD1-04 | 0.623 | 44 | 45 | 48.5 | 13.75 | −0.944 | 0.345 |
| DRD1-05 | 0.623 | 44 | 44 | 48 | 14 | −1.069 | 0.285 |
| DRD2-11 | 0.622 | 43 | 58 | 51.5 | 13.25 | 1.786 | 0.074 |
| DRD2-24 | 0.696 | 42 | 66 | 55 | 12.5 | 3.111 | 0.002 |
| DRD2-25 | 0.554 | 45 | 54 | 50 | 15 | 1.033 | 0.302 |
| DRD2-35 | 0.859 | 25 | 30 | 35.5 | 6.75 | −2.117 | 0.034 |
| DRD2-42 | 0.177 | 29 | 23 | 19.5 | 8.75 | 1.183 | 0.237 |
| HTR1A-21 | 0.504 | 49 | 51 | 48.5 | 15.75 | 0.63 | 0.529 |
| HTR1B-01 | 0.255 | 33 | 22 | 23 | 10.5 | −0.309 | 0.758 |
| HTR1B-02 | 0.741 | 39 | 49 | 49.5 | 12.25 | −0.143 | 0.886 |
| HTR1B-03 | 0.854 | 26 | 39 | 35 | 6.5 | 1.569 | 0.117 |
| HTR1B-04 | 0.498 | 52 | 53 | 46 | 18 | 1.65 | 0.100 |
| HTR1D-02 | 0.085 | 21 | 12 | 10.5 | 5.25 | 0.655 | 0.513 |
| HTR1D-03 | 0.183 | 23 | 13 | 15.5 | 6.75 | −0.962 | 0.336 |
| HTR1D-05 | 0.317 | 37 | 28 | 27 | 11.5 | 0.295 | 0.768 |
| HTR1D-06 | 0.638 | 39 | 48 | 48.5 | 12.75 | −0.14 | 0.889 |
| HTR2A-01 | 0.424 | 43 | 41 | 37 | 15 | 1.033 | 0.302 |
| HTR2A-10 | 0.087 | 25 | 12 | 13 | 6.5 | −0.392 | 0.695 |
| HTR2A-18 | 0.933 | 17 | 31 | 24.5 | 4.75 | 2.982 | 0.003 |
| OPRD1-01 | 0.524 | 50 | 47 | 52 | 16 | −1.25 | 0.211 |
| OPRD1-03 | 0.339 | 44 | 29 | 33.5 | 13.75 | −1.214 | 0.225 |
| OPRD1-05 | 0.634 | 44 | 54 | 52 | 14 | 0.535 | 0.593 |
| OPRD1-06 | 0.859 | 27 | 35 | 38.5 | 7.75 | −1.257 | 0.209 |
| OPRD1-07 | 0.67 | 47 | 56 | 58.5 | 16.75 | −0.611 | 0.541 |
| OPRD1-08 | 0.539 | 45 | 43 | 46 | 14.5 | −0.788 | 0.431 |

TABLE 19
Results of Contingency Table Analysis of AN-ARP Database

| | | | | | | | | | | | | | | | | | TDT |
|---|---|---|---|---|---|---|---|---|---|---|---|---|---|---|---|---|---|
| | | Genotypic C:C with EAF | | | | | | | | Allelic C:C with EAF | | | | | | | probands |
| Gene | SNP | ARP | PBN | AN1 | AN1 | AN2 | AN2 | AN | AN | AN1 | AN1 | AN2 | AN2 | AN | AN | AN | only |
| | | | | <.05 | <.10 | <.05 | <.10 | <.05 | <.10 | <.05 | <.10 | <.05 | <.10 | <.05 | <.10 | <.05 | <.10 |
| ADRB1 | ADRB1-02 | 0 | 0 | 0 | 1 | 0 | 1 | 1 | 0 | 0 | 0 | 0 | 0 | 0 | 0 | | |
| ADRB2 | ADRB2-01 | 1 | 0 | 1 | 0 | 0 | 0 | 0 | 0 | 0 | 0 | 0 | 1 | 0 | 0 | 1 | 0 |
| | ADRB2-02 | 1 | 0 | 0 | 0 | 0 | 0 | 0 | 0 | 0 | 0 | 0 | 0 | 0 | 0 | 0 | 0 |
| | ADRB2-03 | 1 | 0 | 0 | 0 | 0 | 0 | 0 | 0 | 0 | 0 | 0 | 0 | 0 | 0 | 0 | 0 |
| | ADRB2-04 | 0 | 0 | 0 | 0 | 0 | 0 | 0 | 0 | N | N | N | N | N | N | | |
| ADRB3 | ADRB3-01 | 1 | 0 | 1 | 0 | 0 | 0 | 1 | 0 | 1 | 0 | 0 | 0 | 1 | 0 | 0 | 0 |
| | ADRB3-02 | 1 | 0 | 1* | 0 | 0 | 0 | 0 | 0 | 1* | 0 | 0 | 0 | 0 | 1* | 0 | 0 |
| | ADRB3-03 | 0 | 0 | No variation for | | | | | | | | | | | | | |
| | | | | analyses | | | | | | | | | | | | | |
| | ADRB3-06 | 0 | 0 | 0 | 0 | 0 | 0 | 0 | 0 | 0 | 0 | 0 | 0 | 0 | 0 | | |
| COMT | COMT-01 | 1 | 0 | 1 | 0 | 0 | 0 | 0 | 0 | 0 | 0 | 0 | 0 | 0 | 0 | 0 | 0 |
| | COMT-03 | 0 | 0 | 0 | 1 | 0 | 0 | 0 | 0 | 0 | 0 | 0 | 0 | 0 | 0 | | |
| | COMT-04 | 0 | 0 | 0* | 0* | 0* | 0* | 0 | 0 | 0 | 0 | 0 | 0 | 0 | 0 | | |
| | COMT-06 | 0 | 0 | 0* | 0* | 0 | 0 | 0 | 0 | 0 | 0 | 0 | 0 | 0 | 0 | | |
| DRD1 | DRD1-03 | 1 | 0 | 0* | 0* | 0* | 0* | 0* | 0* | 0 | 0 | 0 | 0 | 0 | 0 | 0 | 0 |
| | DRD1-04 | 1 | 0 | 0 | 0 | 0 | 1 | 0 | 0 | 0 | 0 | 1 | 0 | 1 | 0 | 0 | 0 |
| | DRD1-05 | 1 | | | | | | | | | | | | | | 0 | 0 |
| DRD3 | DRD3-01 | 1 | | | | | | | | | | | | | | 1 | 0 |
| DRD4 | DRD4-01 | 1 | 0 | 0* | 0* | 0* | 0* | 0* | 0* | 0 | 0 | 0 | 0 | 0 | 0 | 0 | 1 |
| DBH | DBH-01 | 0 | 0 | 1 | 0 | 0 | 0 | 0 | 1 | 1 | 0 | 0 | 1 | 1 | 0 | | |
| | DBH-09 | 0 | 0 | 0 | 0 | 0* | 0* | 0 | 0 | 0 | 0 | 0 | 0 | 0 | 0 | | |
| GOLF | GOLF-01 | 1 | | | | | | | | | | | | | | 0 | 1 |
| HCRTR2 | HCRTR2-03 | 0 | 0 | 0* | 0* | 0* | 0* | 0* | 0* | 0 | 0 | 0 | 0 | 0 | 0 | | |
| | HCRTR2-04 | 0 | 0 | 0* | 0* | 0* | 0* | 0* | 0* | 0 | 0 | 0 | 1 | 0 | 1 | | |
| SLC6A4 | 5HTT-01 | 0 | 1 | 0* | 0* | 1* | 0* | 0* | 0* | 0 | 0 | 0 | 0 | 0 | 0 | 0 | 0 |
| | 5HTT-06 | 1 | 0 | 0 | 0 | 0 | 0 | 0 | 0 | 0 | 0 | 0 | 0 | 0 | 0 | 1 | 0 |
| HTR1B | HTR1B-01 | 0 | 0 | 0 | 0 | 0 | 0 | 0 | 0 | 0 | 0 | 0 | 0 | 0 | 0 | | |
| | HTR1B-02 | 0 | 0 | 0* | 0* | 0* | 0* | 0* | 0* | 0 | 0 | 0 | 0 | 0 | 0 | | |
| | HTR1B-03 | 0 | 0 | 1* | 0* | 0* | 0* | 0* | 0* | 0 | 1 | 0 | 0 | 0 | 0 | | |
| HTR2A | HTR2A-01 | 1 | 1 | 0 | 1 | 0 | 0 | 0 | 1 | 0 | 0 | 0 | 0 | 0 | 0 | 0 | 0 |
| | HTR2A-06 | 1 | | | | | | | | | | | | | | 0 | 0 |
| | HTR2A-10 | 0 | 0 | 0* | 0* | 0* | 0* | 0* | 0* | 0 | 0 | 0* | 1* | 0 | 0 | 0 | 0 |
| | HTR2A-18 | 0 | 0 | 0 | 0 | 0* | 0* | 0* | 0* | 0 | 0 | 0 | 0 | 0 | 0 | | |
| HTR2C | HTR2C-01 | 1 | | | | | | | | | | | | | | 1 | 0 |
| | HTR2C-02 | 0 | 0 | 0 | 0 | 0 | 0 | 0 | 0 | 0 | 0 | 0 | 0 | 0 | 0 | | |
| HTR5A | HTR5A-01 | 0 | 0 | 0* | 0* | 0* | 1* | 0 | 1 | 0 | 0 | 0 | 0 | 0 | 0 | | |
| | HTR5A-03 | 0 | 0 | 0* | 0* | 0* | 0* | 0 | 0 | 0 | 0 | 0 | 0 | 0 | 0 | | |
| TH | TH-01 | 0 | 0 | 0 | 0 | 0 | 0 | 0 | 0 | 0 | 1 | 0 | 0 | 0 | 0 | | |
| TRH | TRH-04 | 1 | 1 | 1 | 0 | 1 | 0 | 1 | 0 | 1 | 0 | 1 | 0 | 1 | 0 | 0 | 1 |
| | TRH-05 | 0 | 0 | 0 | 0 | 0 | 0 | 0 | 0 | 0 | 0 | 0 | 0 | 0 | 0 | | |
| | TRH-06 | 0 | 0 | 1 | 0 | 0 | 0 | 0 | 0 | 0 | 0 | 0 | 0 | 0 | 0 | | |
| TRHR | TRHR-04 | 0 | 0 | 0 | 0 | 0 | 0 | 0 | 0 | 0 | 0 | 0 | 0 | 0 | 0 | | |
| | TRHR-05 | 0 | 0 | 0 | 0 | 0 | 0 | 0 | 0 | 0 | 0 | 0 | 0 | 0 | 1 | | |

TABLE 20
Results of contingency table analyses of BN probands

| Polymorphism | Mutation | HGBASE ID | Position | Type of polymorphism | Type* | BN vs. XXF |
|---|---|---|---|---|---|---|
| ADRB3-01 | T > C | SNP000000522 | 190 | W64R - Associated with | A | 9.422 (0.009) |
| | | | | Hyperinsulinaemia | G | 9.282 (0.010) |
| ESR1-02 | C > T | SNP000670004 | | intron | A | 9.366 (0.009) |
| | | | | | G | 11.179 (0.004) |
| HTR1B-01 | C > T | SNP000006652 | 129 | silent | A | 8.981 (0.011) |
| | | | | | G | 8.675 (0.013) |
| HTR1B-02 | C > G | SNP000007238 | 861 | silent | A | 7.414 (0.025) |
| | | | | | G | 7.493 (0.024) |
| HTR1D-02 | T > C | SNP000006432 | 1080 | silent | A | 4.591 (0.101) |
| | | | | | G | 7.99 (0.018) |
| HTR1D-03 | C > T | SNP000083091 | −628 | 5′ | A | 10.535 (0.005) |
| | | | | | G | 13.084 (0.001) |

*G = genotypes; A = alleles.
Numbers shown are X² (p).

TABLE 21
TDT analysis of AN probands and parents

| | Allele | b | c | Chi-Sq | W | Mean(A) | Var(V) | z′ | p |
|---|---|---|---|---|---|---|---|---|---|
| 5HTT-06 | 2 | 19 | 34 | 4.245 | 19 | 27 | 13.5 | 2.041 | 0.041 |
| | 1 | 34 | 19 | 4.245 | 36 | 28 | 13.5 | 2.041 | 0.041 |
| ADRB2-01 | 1 | 33 | 18 | 4.412 | 33 | 25.5 | 12.75 | 1.960 | 0.050 |
| | 2 | 18 | 33 | 4.412 | 18 | 25.5 | 12.75 | 1.960 | 0.050 |
| DRD2-25 | 1 | 40 | 19 | 7.475 | 40 | 29.5 | 14.75 | 2.604 | 0.009 |
| | 2 | 19 | 40 | 7.475 | 19 | 29.5 | 14.75 | 2.604 | 0.009 |
| DRD2-43 | 2 | 13 | 2 | 8.067 | 13 | 7.5 | 3.75 | 2.582 | 0.010 |
| | 1 | 2 | 13 | 8.067 | 2 | 7.5 | 3.75 | 2.582 | 0.010 |
| DRD3-01 | 2 | 35 | 16 | 7.078 | 37 | 27 | 13 | 2.635 | 0.008 |
| | 1 | 16 | 35 | 7.078 | 16 | 26 | 13 | 2.635 | 0.008 |
| HTR1D-03 | 1 | 7 | 18 | 4.84 | 7 | 12.5 | 6.25 | 2.000 | 0.046 |
| | 2 | 18 | 7 | 4.84 | 18 | 12.5 | 6.25 | 2.000 | 0.046 |

TABLE 22
TDT analysis of BN-ARP dataset

| SNP | test | p | SE* |
|---|---|---|---|
| CCK-01 | | | |
| alleles | Permutation McNemar (MCMC) | 0.098 | −0.002 |
| | Asymptotic McNemar (X\hat{ }2) | 0.070 | |
| | Asymptotic Marginal (X\hat{ }2) | 0.070 | |
| | Permutation Marginal (MCMC) | 0.050 | −0.001 |
| genotypes | Permutation McNemar (MCMC) | 0.234 | −0.005 |
| | Asymptotic McNemar (X\hat{ }2) | 0.210 | |
| | Asymptotic Marginal (X\hat{ }2) | 0.145 | |
| | Permutation Marginal (MCMC) | 0.148 | −0.005 |
| DRD2-11 | | | |
| alleles | Permutation McNemar | 0.019 | −0.001 |
| | (MCMC) | | |
| | Asymptotic McNemar (X\hat{ }2) | 0.015 | |
| | Asymptotic Marginal (X\hat{ }2) | 0.015 | |
| | Permutation Marginal (MCMC) | 0.020 | −0.001 |
| genotypes | Permutation McNemar (MCMC) | 0.164 | −0.006 |
| | Asymptotic McNemar (X\hat{ }2) | 0.167 | |
| | Asymptotic Marginal (X\hat{ }2) | 0.099 | |
| | Permutation Marginal (MCMC) | 0.106 | −0.004 |
| DRD2-24 | | | |
| alleles | Permutation McNemar | 0.009 | 0.000 |
| | (MCMC) | | |
| | Asymptotic McNemar (X\hat{ }2) | 0.007 | |
| | Asymptotic Marginal (X\hat{ }2) | 0.007 | |
| | Permutation Marginal (MCMC) | 0.010 | 0.000 |
| genotypes | Permutation McNemar | 0.021 | −0.001 |
| | (MCMC) | | |
| | Asymptotic McNemar (X\hat{ }2) | 0.026 | |
| | Asymptotic Marginal (X\hat{ }2) | 0.008 | |
| | Permutation Marginal (MCMC) | 0.008 | 0.000 |
| HTR1B-03 | | | |
| alleles | Permutation McNemar (MCMC) | 0.061 | −0.001 |
| | Asymptotic McNemar (X\hat{ }2) | 0.048 | |
| | Asymptotic Marginal (X\hat{ }2) | 0.048 | |
| | Permutation Marginal (MCMC) | 0.066 | −0.002 |
| genotypes | Permutation McNemar | 0.008 | 0.000 |
| | (MCMC) | | |
| | Asymptotic McNemar (X\hat{ }2) | 0.012 | |
| | Asymptotic Marginal (X\hat{ }2) | 0.051 | |
| | Permutation Marginal (MCMC) | 0.047 | −0.002 |
| HTR2A-18 | | | |
| alleles | Permutation McNemar | 0.007 | 0.000 |
| | (MCMC) | | |
| | Asymptotic McNemar (X\hat{ }2) | 0.005 | |
| | Asymptotic Marginal (X\hat{ }2) | 0.005 | |
| | Permutation Marginal (MCMC) | 0.007 | 0.000 |
| genotypes | Permutation McNemar | 0.016 | −0.001 |
| | (MCMC) | | |
| | Asymptotic McNemar (X\hat{ }2) | 0.023 | |
| | Asymptotic Marginal (X\hat{ }2) | 0.007 | |
| | Permutation Marginal (MCMC) | 0.009 | 0.000 |

TABLE 23
Analysis of TDT in BN probands and parents only.

| SNP | A1 freq | # inf fam | S | E(S) | Var(S) | Z | P |
|---|---|---|---|---|---|---|---|
| DRD2-24 | 0.696 | 42 | 66 | 55 | 12.5 | 3.111 | 0.002 |
| DRD2-35 | 0.859 | 25 | 30 | 35.5 | 6.75 | −2.117 | 0.034 |
| HTR2A-18 | 0.933 | 17 | 31 | 24.5 | 4.75 | 2.982 | 0.003 |

TABLE 24
Results from Contingency Analyses of Pairwise Haplotypes.

| | (case control; bottom left AN) | | | | | |
|---|---|---|---|---|---|---|
| | DRD2-43 | DRD2-11 | DRD2-24 | DRD2-25 | DRD2-35 | DRD2-42 |
| DRD2-43 | | | | | | |
| DRD2-11 | 7.988 | | | | | |
| | (0.046) | | | | | |
| DRD2-24 | 9.657 | 1.621 | | | | |
| | (0.022) | (0.655) | | | | |
| DRD2-25 | 12.183 | 0.168 | 1.337 | | | |
| | (0.007) | (0.919) | (0.512) | | | |
| DRD2-35 | 11.196 | 1.080 | 8.454 | 1.654 | | |
| | (0.011) | (0.583) | (0.038) | (0.437) | | |
| DRD2-42 | 4.780 | 1.026 | 1.266 | 2.154 | 1.013 | |
| | (0.092) | (0.795) | (0.531) | (0.541) | (0.602) | |

1. An isolated nucleic acid molecule comprising a variant gene associated with an eating disorder selected from the group consisting of the polymorphisms in Table 1.

2. An isolated nucleic acid molecule of claim 1, wherein the eating disorder is anorexia nervosa.

3. An isolated nucleic acid molecule of claim 1, wherein the eating disorder is bulimia nervosa.

4. An isolated nucleic acid molecule of claim 2, wherein the gene is a HTR1D variant selected from the group consisting of HTR1D-05, HTR1D-03, HTR1D-07, and HTR1D-06.

5. An isolated nucleic acid molecule of claim 2, wherein the gene is an OPRD1 variant selected from the group consisting of OPRD1-06, OPRD1-01, OPRD1-03, OPRD1-07 and OPRD1-05.

6. An isolated nucleic acid molecule of claim 2, wherein the gene is a DRD2 variant selected from the group consisting of DRD2-43, DRD2-11, DRD2-23, DRD2-24, DRD2-25, DRD2-35, and DRD2-42.

7. An isolated nucleic acid molecule of claim 2, wherein the gene is a variant selected from the group consisting of ADRB1-02, ADRB2-01, ADRB2-02, ADRB2-03, ADRB2-04, ADRB3-01, ADRB3-02, ADRB3-03, ADRB3-06 COMT-01, COMT-03, COMT-04, COMT-06, DRD1-03, DRD1-04, DRD1-05, DRD3-01, DRD4-01, DBH-01, DBH-09, GOLF-01, HCRTR2-03, HCRTR2-04, 5HTT-01, 5HTT-06, HTR1B-01, HTR1B-02, HTR1B-03, HTR2A-01, HTR2A-06, HTR2A-10, HTR2A -18, HTR2C-01, HTR2C-02, HTR5A-01, HTR5A-03, TH-01, TRH-04, TRH-05, TRH-06, TRHR-04, and TRHR-05.

8. An isolated nucleic acid molecule of claim 1, wherein the variant comprises at least one single nucleotide polymorphism relative to GenBank Accession No. AL353585, AF050737, or U07882.

9. An isolated antibody that specifically recognizes a receptor variant of claim 1.

10. A vector comprising an isolated nucleic acid molecule of claim 1.

11. A host cell transformed to contain the nucleic acid molecule of claim 1.

12. A host cell comprising a vector of claim 10.

13. A host cell of claim 12, wherein said host is selected from the group consisting of prokaryotic hosts and eukaryotic hosts.

14. A method for producing a polypeptide, comprising the step of culturing a host cell transformed with the nucleic acid molecule of claim 1 under conditions in which the protein encoded by said nucleic acid molecule is expressed.

15. The method of claim 14, wherein said host cell is selected from the group consisting of prokaryotic hosts and eukaryotic hosts.

16. A method of identifying an agent which modulates the expression of a nucleic acid molecule encoding a serotonin receptor 1D, a delta-opioid receptor, or a dopamine receptor D2 of claim 1, comprising the steps of:
exposing cells which express the nucleic acid to the agent; and
determining whether the agent modulates expression of said nucleic acid, thereby identifying an agent which modulates the expression of a nucleic acid encoding a serotonin receptor 1D, a delta-opioid receptor, or a dopamine receptor D2.

17. A method of identifying an agent which modulates at least one activity of a serotonin receptor 1D, a delta-opioid receptor, or a dopaamine receptor D2 of claim 1, comprising the steps of:
exposing cells which express the protein to the agent; and
determining whether the agent modulates at least one activity of said receptor, thereby identifying an agent which modulates at least one activity of a serotonin receptor 1D, a delta-opioid receptor, or a dopamine receptor D2.

18. The method of claim 17, wherein the agent modulates one activity of the protein.

19. A method of diagnosing a genetic predisposition to an eating disorder in a subject, comprising detecting the presence or absence of one or more single nucleotide polymorphisms in a nucleic acid sample derived from the subject, wherein the single nucleotide polymorphisms are selected from a group consisting of the polymorphisms in Table 1.

20. The method of claim 19, wherein the eating disorder is anorexia nervosa or bulimia nervosa.

21. A method of claim 19, comprising detecting the presence or absence of a nucleotide polymorphism at a position corresponding to the single nucleotide polymorphism of variant HTR1D-05, HTR1D-03, HTR1D-07, or HTR1D-06.

22. A method of claim 19, comprising detecting the presence or absence of a nucleotide polymorphism at a position corresponding to the single nucleotide polymorphism of variant OPRD1-06, OPRD1-01, OPRD1-03, OPRD1-07, or OPRD1-05.

23. A method of claim 19, comprising detecting the presence or absence of a nucleotide polymorphism at a position corresponding to the single nucleotide polymorphism of variant DRD2-43, DRD2-11, DRD2-23, DRD2-24, DRD2-25, DRD2-35, or DRD2-42.

24. A method of claim 19, comprising detecting the presence or absence of a nucleotide polymorphism at a position corresponding to the single nucleotide polymorphism of variant ADRB1-02, ADRB2-01, ADRB2-02, ADRB2-03, ADRB2-04, ADRB3-01, ADRB3-02, ADRB3-03, ADRB3-06, COMT-01, COMT-03, COMT-04, COMT-06, DRD1-03, DRD1-04, DRD1-05, DRD3-01, DRD4-01, DBH-01, DBH-09, GOLF-01, HCRTR2-03, HCRTR2-04, 5HTT-01, 5HTT-06, HTR1B-01, HTR1B-02, HTR1B-03, HTR2A-01, HTR2A-06, HTR2A-10, HTR2A-18, HTR2C-01, HTR2C-02, HTR5A-01, HTR5A-03, TH-01, TRH-04, TRH-05, TRH-06, TRHR-04, or TRHR-05.

25. A method of diagnosing a genetic predisposition to an eating disorder, comprising detecting the presence or absence of a serotonin receptor 1D variant selected from the group consisting of a variant comprising a guanine at a position corresponding to nucleic acid position 2190, a variant comprising a thymidine at a position corresponding to nucleic acid position 1080, a variant comprising a cytosine at a position corresponding to nucleic acid position -628 and a variant comprising a cytosine at a position corresponding to nucleic acid position -1123.

26. A method of diagnosing a genetic predisposition to an eating disorder, comprising detecting the presence or absence of a delta-opioid receptor variant selected from the group consisting of a variant comprising a guanine at a position corresponding to nucleic acid position 80, a variant comprising a guanine at a position corresponding to nucleic acid position 47821, a variant comprising a thymidine at a position corresponding to nucleic acid position 51502, a variant comprising a cytosine at a position corresponding to nucleic acid position 8214 and a variant comprising an adenosine at a position corresponding to nucleic acid position 23340.

27. A method of diagnosing a genetic predisposition to an eating disorder, comprising detecting the presence or absence of a dopamine receptor D2 variant selected from the group consisting of a variant comprising a guanine at a position corresponding to nucleic acid position 932, a variant comprising a thymidine at a position corresponding to nucleic acid position 957, a variant comprising a thymidine at a position corresponding to nucleic acid position 14664, a variant comprising a thymidine at a position corresponding to nucleic acid position 24490, a variant comprising a cytosine at a position corresponding to nucleic acid position 939, a variant comprising a cytosine at a position corresponding to nucleic acid position 2739, and a variant comprising a cytosine at a position corresponding to nucleic acid position -141.

28. An allele-specific primer that detects a polymorphism in the gene encoding a serotonin receptor 1D associated with an eating disorder.

29. An allele-specific primer that detects a polymorphism in the gene encoding a delta-opioid receptor associated with an eating disorder.

30. An allele-specific primer that detects a polymorphism in the gene encoding a dopamine receptor D2 associated with an eating disorder.

31. An allele specific primer of claim 28, wherein the eating disorder is anorexia nervosa or bulimia nervosa.

32. A kit comprising a primer of claim 28.

33. A solid support comprising at least one oligonucleotide capable of specifically hybridizing to one allele of a polymorphism selected from a group consisting of the polymorphisms in Table 1.

34. A solid support according to claim 33, comprising at least 3 different oligonucleotides, wherein each oligonucleotide is capable of specifically hybridizing with one allele of a polymorphism selected from a group consisting of the polymorphisms in Table 1.

35. A solid support according to claim 33, comprising at least 5 different oligonucleotides, wherein each oligonucleotide is capable of specifically hybridizing with one allele of a polymorphism selected from a group consisting of the polymorphisms in Table 1.

36. A solid support according to claim 33, comprising oligonucleotides capable of specifically hybridizing to one or more polymorphisms selected from a group consisting of HTR1D-05, HTR1D-03, HTR1D-07, and HTR1D-06.

37. A solid support according to claim 33, comprising oligonucleotides capable of specifically hybridizing to one or more polymorphisms selected from a group consisting of OPRD1-06, OPRD1-01, OPRD1-03, OPRD1-07, and OPRD1-05.

38. A solid support according to claim 33, comprising oligonucleotides capable of specifically hybridizing to one or more polymorphisms selected from a group consisting of DRD2-43, DRD2-11, DRD2-23, DRD2-24, DRD2-25, DRD2-35, and DRD2-42.

39. A solid support according to claim 33, comprising oligonucleotides capable of specifically hybridizing to one or more polymorphisms selected from a group consisting of ADRB1-02, ADRB2-01, ADRB2-02, ADRB2-03, ADRB2-04, ADRB3-01, ADRB3-02, ADRB3-03, ADRB3-06, COMT-01, COMT-03, COMT-04, COMT-06, DRD1-03, DRD1-04, DRD1-05, DRD3-01, DRD4-01, DBH-01, DBH-09, GOLF-01, HCRTR2-03, HCRTR2-04, 5HTT-01, 5HTT-06, HTR1B-01, HTR1B-02, HTR1B-03, HTR2A-01, HTR2A-06, HTR2A-10, HTR2A-18, HTR2C-01, HTR2C-02, HTR5A-01, HTR5A-03, TH-01, TRH-04, TRH-05, TRH-06, TRHR-04, and TRHR-05.

40. A non-human transgenic animal modified to contain a nucleic acid molecule of claim 1.

41. The transgenic animal of claim 40, wherein the nucleic acid molecule contains a mutation that prevents expression of the encoded protein.

42. A database comprising SNP allele frequency information on one or more SNPs identified as associated with eating disorders, wherein the database is on computer-readable medium.

43. A database according to claim 42, wherein the SNPs are selected from a group consisting of the polymorphisms in Table 1.

44. A database according to claim 43, further comprising information on one or more factors selected from a group consisting of environmental factors, other genetic factors, related factors, including but not limited to biochemical markers, behaviors, and/or other polymorphisms, including but not limited to low frequency SNPs, repeats, insertions and deletions.


SEQUENCE LISTING (98 sequences)

SEQ ID NO: 1 - DNA, 24420 bp, Homo sapiens
  misc_feature: AL353585
acctagcccaagctttgaattcttccttactctcttccccagcacttaaccagttatttcataaaaacaataagggttgggaggccgaggctggcggatcatgaggtcagatcaagaccatcctggccaacatggtgaaaccccgtctctactaaaaacacaaaaattagctgtgcgtggtggtgtgcgcctatagtcccagctacttgggaggctgaggcaggagaatcgcttgagcctgtgagacagaggttgcagtgagctgaaatcgcaccattgcactccagcctgggcgacagagacagactccgtctaaaaacaaaacaaaacaaaaacaataaggaagcagaggctcacatatcaatggcaaagtctagggtctttctcttaataggccagacctttcactcccctacacattctgtattctctcccttgtcttcactaaacatgtttggggcattcatgtcttagttcaggctgttccatctgcctagaagagcaatctttcccattccctttcatacatatctgttgtactcctgtctgctttctaaattttttttgtttgttttttagagatggggtctcatcatgttgcacaggctaatctcaaactcctgggctccagcagtttgcccacctgggcttcccaaagtggtgggatacaggtgtgagccacgctacccagcccctctctctgctttctgaggtccttttcaaaagctacctcctccaggaagtcttccctggacaactgagtgcaaaatctctctcctttaagccctctcccgactccaggtttgtttgtgtctctcattgcctactgctggaagttccttgaagatttacctattttcttcccactccagtgttcagagcacagtagactggcaaataataggaaataacttctcttgtaaggctcaagtaggccctggagaaagatcagtatttcctcttctccatttaaagtttttggctgggtgcaatggctcacgcctgtaattccaacacttcgggaggctgaggtaggaggatcacttgagctcaggagttcaagaccagccagggcaacatggcgaaaccccatctctacaaaaaatacaaaaattagctgggcatgatggtgcgtgcctgtagtcccagctactctggaggctgaggtggaaggatgtcttgagcccaaaaggtggaagttgcagtgagcccagagtgcactgctgcactctagcctgggtgacaaagccagactctgtcatcattcattcattcattcattcataaataaatacatttctccaacagctcccacttcactctgaataaaattcaaaactcccatggcttacaaggccctacgtgatctgctgccaccccggccgtctctctgacctcatctcctaccagctgcccgtcttaaatatcacccctttggcaacaccttgaccagccaacataaagcagctcccctcctccaccacatcaccctggtttatgctaattttagcacttaacctgatctgatattttctgcctgtttgtttattgcgtatctttccccacacaccctccacctgccccatgccctgctaacatacacctcctgtctagctcatccctatatccccagagcctagaacagtgcctggctgaaaataaataggtgggtcaacagcctgaggagcgatctgggctagaggcatacatgtatgagtcatgtgcatagaggtgataactaaagccaatggggtgggtgagatcacccagagagagtgtctactaggagaggagcagagggcctgggggagcagagcatctaagggctgtagaaaggagaatgaagccgcaaaaagatgagggaacaacgtaacagtgtcaagtcacaggggcccagagaagagagtgttttggggagaagaaacagtcagcagtgtcaactgctgctgagagcagtgaaagactgagcaggccaggctccatggctcatacctgtagtcccagcattttgggaggccaaggagggtggattctttgagcccaagagttcgaggccagcctgggcaacatggcgacaccctgtctctacaaaaaatgcaaaaattagccaggcatggagtcgcatgcctgtagtcccagctactggggaggctgaggcaggaggatcacttgaactcaggaagttgaggttgcagtgagccatgatcacaccactacactccagcctgggtgacagagcaccaccctgtctcaaaaacaaaaacaaataagcaaaaagactgagtggtgtctgctgaattcagcttcctggaagacatggctgacccctgcccctctgtgggccttagacgggaaacatctgtgcaatgaaagagacctctcaggtctgggaactccatactctgagaatctgtatttctgctcacattcacattactctcaccaaaaagaatagtcatccatccgtggcctaggagagaagggatgagccagtggtcccgccctcattattgatccatcaggtaactgacaaactctaaggaagcatctctgtttttctggccctgtactaggttctggaaggcggtgagccagcaggcaggcctgggactgggaagccagcactagggctcagggcttctgtggctgcagagacatgatctccatcccccacccacgggctgcactgggacttacttgttcatgagatcaaagccttggtctgacagcagggccctgaagtgcttgtagaggttgttgtaggggtagaggccctagcccctgccagtcaagtccagctggcctggtccctccctcctagccaccctctaggtcccagcttcctggatgtctcaatgacaaaggctgcctacttctccccaccagtggagcaggactaaagactgaaaaaggctggctaccccaatggcctcacttgctgctgataaagtgtccccaaatgtccttgtgaggagggctgagaccccagccagtggctgtgtgatccagaggacagcgcagacccacaccacgggctcaactggaaaacactggaagctggttgcaaggaagagtggaggggttgctgctgctgctactcagagaaatgatggccactagtggctgagctctggtttggagccaggggctgtgctgagtgacaatcccattagatttgtacagtaagcctgtggggtgagaattgttgtccccacttcatatgggaggaaaccacacctcaaagaaggtaagggctttcctgagatcacacagctagtaggtggccaaggccagtttgctaatgcaaccgggaccctgactgagagccctgctcttaatcccgaactttcccagacctggggactttagtcttagattcttctactggtagtgtgaccttgggcaagacggattccccactctccccatttgtaataaagcagatttctcagtgcccttccagttccggcattatgtgattagctaggatttatacagaactttacagtttctaaaaaaagcatctagcggggatctcacttatttccagggaccctgcaaagtgagagaatgcccctctatttgacagacgtgcaaactgaggcttggagagatgcagtgacctgcccaaggtcacagagccggtcagccccagcccggagccagccggggctccacaccggacccccttcaactctctccagagaggggaggcccctccgcggcgccgcgcccgaacctggccagcagggggcagcgcccggcttcccttggcgcccggggcccctcggggggcgtcgcggtccccggcccgggggtgcttcctgcccctccagttctcgcctgggcgctgggcacccccaacgcgcgcgtgggatcccgcagcctccctgggcgctgcctcagtcctcccttttctcccggggctctggacggccacccccggtggcctccctccggccgggcaggtcctccgggaccctctccctggcgcgcgcccgtccgagggcacagagaggcgggacgccccgggcccccgaggccccaggaggggcgccgctcccggcctcagttccccgcggacggccgctggggccaggctgcagacgcggccccgagagcttacccgctgcccggcgggcaggtgcgcacacccggcgtacccgcccgatccacttcctcgcgcggcgtctccccgtcgcggccgccttgtctccgccgcgacccccgccgaactcgggggccgcccgccccgccgccccggggccctttccggctcgcgccctcgcgatcccgcacctgcctccgcctctcccagagccctgcggctccttctccgcgctccccttcccgcgcccgccgctcctccttgctccctcctccagctcctccctcgccgggctccctccctactcggccccccgcccgcccggaggaaggcttggcccccggcctccccgctcgggatggtccggagctccgagccccggcctgcgactcgtagcagcccccgcccagcacccggggtgggtacgcgcagcggaagggaggtggaaaaggccccgggccagcgctctccggcccccacctgtaggagggcaggtgggctggctctggggtaggggtctgcagggaggtgggccatgcccagagacacttggtgaaagtccagcctcccctatccgtccgggtccccggtgttcattcctagttggatttttgttttcttcgtctaacaaatattgattgatctactgtgtcaggcatgttctaggcactggggatacagcagtgaacaaaacagaggtagacctaagggagcttccgttctgttgcggggagacaacacatttaatacgcaaaatgcataaaatatttatagaggtaaagggtagggaggataagaaagcagagaaagaaggtagggagtggaagtgagggctcaggggcacctccctgcaaaagtagcatttgagcaaagttaggaggtgaaggagtgggtcttgggaaagaccctgagccggatggtgtccaagctggatgaatggaagggggcggggggggggggcggtcagagaggtaggggagggacagctcccagcatccttcaaaggacagattccgagtgccagcccccagcccccagcaggtgtttgggatgctgagatgaaccagcctggttgcagagaaccaggggagagagaagacttgagcacaaagacaggaccagcaagaggcttccactgaagacagtgaagaggcacgaacaccggccatggcttgtgtccaggtttctcgcagctcatgaggaactggccttgccttctgtgtccttaccggttttttggttggttggttggttggttggttggttggtttggagacggagtcttgctctgtcgcccaggctggagcgcagtggcgcgatctcggctcactgccacctccgcctcccgggttcaagcgattctcctgcctcagcctcctgagtagctgggactacagaagcgtgccacctgcccggctaattttttgtatttttaatagagacggggtttcaccgtattagccaggatggtctgcatcttctgacctcgtgatccgcccgcctcggcctcccatagtgctgggattacaggcatgagccactgcgctcagctccttactgggtctttatctgcttgttcttcatgtgttttgaagcctgttattagttaggggcattaagattgttatgttatgtttgtttgttttttgtttttgtttgtttgtttgttttttagagacggggtcttgctatgttgttcaggctggtttcaaactcctgggctcaagtgatcctcccgccttggcctcccaaagtgctgagattacaggtgtaagccactgtggccaaccctgttatttcttcttgatgaattgacccttttatcattacgaaatgaccctctgtccctggtaacattctttgtcttgaagtctccttcactgggtgtagggagaggccagcagcacctgccggcattggtttattcgcagcccacacagctgttggacatctggcacacccaaaactcagcagctcatctagactttctattctgtgtccttctgtgtccttctgtctccacataactctctacaaacctagccctcccctcctttcttcatgccccaccctcttggtttaccatttgatgattggcctggagcctacatctctctcaatctctctcttttttggaagcctgtgatcaatgatcattaataatgcaaacattcttttcgtcttagcacaaatcccaggcaaataaatcactggcaaataggagttatcggcaaccacacctcggcctccactctactcaggccctaggttcttttcccaaataggaaataggaaggttgagcaatgaaaaatgtaggcatttcccaaaataggcactgcggctcatgccccaggttgaagtcaggaaggaggaaagggtcctgtctacacagccgaagcacttgcagctgctgttggggtcctagcctaagatcctgcagttgaaaaatccaaatggcagagtaaatacagacacatcctgggtgtgtgtcctcaatgaaaagaccctgacagccccagaagctgccctggtcagggcgaaccttgggcaaaacttgtatggcggccgggcgcggtggctcacgcctgtaatcccagcactttgggaggccgaggcgggcagatcatgaggttaggagatcgagaccatcccggctaaaacggtgaaaccccgtctctactaaaaatacaaaaaatagccgggcgtagtggcgggcacctgtagtcctagctacttgggaggctgaggcaggagaatggcgtgaacccaggaggcggagcttgcagtgagccgagatcgtgccactgcactccagcctgggcgacagagcgagactccgtctcaaaaaacaaaaacaaaaacaaaaacaaaaaaaaaacttgtatggcatctcagcaccttctaacaaaacatctgctaacaatgttgaacaatacaagccactgaagatggtaaatgacactggaaagctggctgaaggaaaaggctgggtctgctggagagggttcacttcagcgtctgagcccttaccctgggcccgtggatgtgttaagcacagtccctgccctcaagaagctcacaatccccaagggaaaacccacacacagttaagcatagcacagtggggaagaccaggagagaggtagcagggggcccacggggctcagagtggagcacctgggaggtgggcaagcagtcaggaagcagctgggaagggtacccctgtgctgagtcttgaaggatgagtgggaggcatctaagcaaagaaagagcaagggacgtggagaaattccctggcagaaggaatatttgtgcaaaggcatggaggctaaagagaattcagttagggccactgcaagtcttttggtattactagagttgtggagagtggttgacacgaggctggaggagtaggaaggggccagcacgtgaagagccttgaatgctaggaaaagaagctgggctttatccttagagtgatgggagccagggaagggctttgagcaagggaatgacatgattagacaggacagattgctaatccctgatgccctgaaacttaatactcaatttttcctatcaattgcccaagatccctcttaaattataaccaatatatttatttgaaagcttggtttttctcagtttcttaccctatatatgcatctcaaaaatgtgtaaagtactccagcatcaggggaaaagcacaaaactctaggaacttgtatgtgtgaaacaaagtggctcattaaaagctcagaattattttgaaatctcatttttatttttaaaatctacaaaattttttgttttactatatcaatgtttaatagaaataaaatatttcaagttactcaccattgaataaacttgacactattggggaaaaaaaactataagtccagtgatcagactgggtctgaatctcaattttgctgtttaatagctgtgcaatcctggacaagttatctaaattctgcgagcctcattctgcccatctgtaaaatggtcacaattaagcatacctatctcatggggttagttatgatgagggccacgttgaaatcatgcatctgaagtacacaacacaggaccttgcacacaggaagctcttcaggagtattccccatgactctacttatagttgggatgccacacttttcacaatgattttgtgtttacctcccccaatattatgagtttctagaggccagcttgtgtcttactcacctaggtatccccagtgcctagcactgaatctggtacattgtaagcattcaaccaaggatggatggatggatggatgaatggatggatggatggataaattaatgaaaaaagaagacaccccgccactcctatgcccatagagtgcccctctgcttcagaggtgggagtcaggcttattcttttttaaagagggttaaatgaatcgtgtgtctggggacaggtgtctggggcaagctttgtcattagacagtctgtggtttgcagtttcttcaggagagagctgtctagacagaagggaggaacaaggactgcatgggaatcaggatcctggggagggcttctcagggaatgggaagcccgggcttctcgctctttttttctttctcatcactcaggatctggctgagaaaatatgctacctgcccctagcagtggaaataggttcatagcttttacctgaggatgacaaagaggggtacagcaaacccctcactagctagcaacagggtaaagttttttgtttgtttttgtttttgttttgagacagagtctcattgtgttgtccaggctggagtgcagtggtgcgatctcagcccactgcaacctctgcctcccaggttcaagtgattctcatgcctcagcctcccgagtagctgggattacagacacgtgccatcacgtctggataaatttttgtatgttagtagagatggggtttcaccaggttgcccaggctggtcttgaactcctgagctcatgtaattcacccgcctaggcctcccaaaatcctgggattacaggtgtgagccaccatgcctggcctttgtgtgtgtgtgtgcatgtgtgtgtatgtgtgtatgtgtgcgccaagcactgcactaagggcctaacatgcattctcctctccattgccctctggggaaagtactattgtgtatttccactctatagatgaaggaacagactcagagattaagtaactttctcttgcaagggtagtaacaggacaaggtcctcagttacatcagactccccatgaatcaggtatctgtctagtcaacaaataatttgagcatcttctctttacacattgctgtggcaagtggggtgggccagtaaggataagtaagatgtgatttctactttgaagaagtttctaacttgaagacggagatgaaggcttccttatctttgtattcccagttcagggcctggcaatacagtgactgttgaataaagcacaggtcgttagagctagaagggtctttggagtttgagtgtgttaatcctgttaatttacagttgggaaaactgagacctagaaggaggaattacctggaaaacacagttaggagaggagcttgggctctgcagccagcctacttagtttgaatctcagctctaccacttattagctgtgtgactttgagttggtcacttaacctctctgattctcagttttcttgtccaagaaagagggaataaagttgtgagaattaagtgagatcatccatgcttagggtttagtatcatcctcattattattattgttatttgagatggggtcttattctgtcacccaggctggggtgcagtggcacgttctcagctcactacaacctccgcttcctgggctcaagtgattctcctgcctcagcctcctgactaactggaactacaggcacacgccagcatgcctagctaattttttatatttttggtagagatggggttttgccatgttgcccaggctggtctggaactcctgagctcaagtgatctgcccaccttggcctctgaaagtgctgggattacagacgtgaaccactatgcctggcccattattattatcattattttattttatttttttgagaaggggtattgggggaacctgcccccaatatttcagcataggttctttctattttccctaagtgttggctggcctgagaaataaagaggaagagtacaaagagaagagttttacagctgggccactgggggtgacatcacttatcagtaggtctctgatgctcacctgagctgcaaaaccaacaagtttgtattagggatttcaaaaggggagagggtgtacgaacagggagtaggtcacaaagatcacatgcttcaaagggcaaaaagagaacaaagatcacatgcttctgaggaaacagggcaaggacaaaagcaaagatgacaaggcaaagggcaaaattagaattactgatgagggtctatgttcagctgtgcacatattgtcttgataaacatcttaaacaacagaaaacagggtttcagagcagagaaccagtctgacctcaaattcaccagggtggggtttttttcctaccctaataagcctgagagtactgcaggggaccagggcatatttcagtccataggacagacactctcagagtgggcatttatagacctcctcccaggaacgcaattcttttcccagagtattaattatcaatattccttgctgggaaaagaatttagcaatagctctcctacttgcacatccgtttataggctctctgcaagaagaaaaatatggctctttttgcccgaccccgcaggcaggcagaccttatggttgtcttcccttgttccctaaaaattgctattattctgttctttttcaaggtgcactgatttcatattgttcaaacatacatgttttaccatcaatttgtacagttagatacaattatcacagtggtcccgaggtgacatacatcctcagcttacgaagataacaggattaagagattaaaataagacaggcgtaagaaattataagagtattatttgggaagtgataaatgtccacattaaaatgaaatcttcacaatttatgttcagagattgaagtaaagacaggcataagaaattataaaactcttaattttgggaactgatatatgtcaatattaaaatgaaatcttcacaatttatgttcttctgccatggctccagtcggtccctctgtttggggtccctgacttcccgcaacaaaggggttttactctgtcatccaagctggaatgcagtggctcaatcatagctcactgcaggctcaaactcctaggctcaaacaatcctcctgcctcaacctcccaagcagctgggactacaggcacatgccaccacaactggctaaattttaaattttatgtagaaataggatctccctatgcccaagctggtctcaaactcctggactcaagagatcctcccatctcagcttcccaaagtgtaggattacaggcatgagccaccacacccagcatgtgtggggtttagcataacgcttagcacacaatcccgcaatacattttgctctgattactattaaacaattgcatagtgagttagtaactcagggcttcctgactcccagtccagtgctctttccagttccccatggttcctgcatctactggagtataatcaggggaggtgtgggggtggtatgtgtgtgatcacatgtccgctgtccttggggacaagtgccttgtgtcccccagcccatatctcagtgcctctcgccatgcctcttctctgtctttgaagccttccccgttctccccccttgaccagagtccagagactcaaccctaaatgaaacacgcaatgaaatgcctgataacatcctttcacctcccattgcaatttcctctgggccagatggggtgaggctgagcctgggaaggagccaacaggaaattcccggggagcagcaagggatgggtgtccaagggcagtcagtgataccatctccagcatcgagcctctgagccaggaagagacaagtctcgcaggagaggttttgctgtttgttttcctcgggcttgacttctaacgagagaatttaattaagagctcgattgcctcgcctggggaataggaatccgaggggatggcagagctgaggccggtggccgaggggaagggagggaggggcctctttggactcagattgaactgtgatgggcttaaactgccccagagaagttcggctcacaggtcttttggtctaatcccaaaacaagattcttctcctacaggaacttgtaagttttagattctgtggtgcaaattaaaagacatttaaaccagtcaaggaaacaggcaaagtttgatattcccaattgttcccactcacactgttaaagaaaaaattattcttattaataataaggcaatggtgggctgggcatggtggctcacacctgtaatcccagcactttgggaggccgaggcaggtggatcacttgaggtcacgagtttgagaccagcctggccaacatggtgaaactctctcacctgggcctcccaaagtgctgagattgcaggcatgcaccacaccatacccggttcccccggacctagaaggaggcctgcacacattagacccacagtaagtattatttcctgaatgaataaaataatataaagcaggcttattagtttcctcacaggactgtgtgaggtttccagtttgttcacaacccaactcctccctcccctgctcatctgcgaaatgagcatagtaaaagtatttacctgtgttggcaggcgcatgtgaaaccatgccggtcaagtgtttagcatgggccaggtgcatagtaagtgccccagaaaaatcagggggttgaatagacagtaaaggtttactccacacttgtgtgtgatatggagaaaactccagaactcaggtataggatgcaggaagacaagggtctgcaatgggggagtccaggcaggaggctgtcgcgagaggtgtgggtcctgactccaggcagtggtgtggggaaaaagatgaggagacaaaaattaggactatttctgagaagttcactagttttttgtttgtttgttttgttttgttttgttttttgagacagagtcttgctctgtcaccccggctagagtgggtgatctcggctcactgcaacctccgtctcccggattcaagcgattctcctgcctcagcctcccagattcaagcgattctcctgcttcagcctcccaagtcgctgggactacaggtgtgcattacaatgcccagctaatttttgtatttttagtagagaccgggtttcaccatgttggccaggctggtctcaaactactgacctcaagtgatccccccacctcggcctcccaaagtgctggggttacaggcatgagctaccatgcctggctgaagtccgctaggttctaactggactgatcccagacttagggagagacacagagagcatcctcagataaccccaccaccttccagtcttgtaagtcaccccccatgtattgggctaacagagtattccaggacaacactcctctgaacatttcaatcaataagagaaggcactgaggtcactcagggaaacagaggaagctaggaagttgcaagcactgcttccctctctgctatgttcttccgcaccaggcacagcacttaagtaattccttaattaatctatataacagcctttggggaggggattatcatgccccatgcccattttatggatgaacaatctgaggctcagaggactgaaaaagctctccccaggttatctcggggagctgcaaaacaacaacaacaacaacaacaacaacaagagccacctgttaacaggggattactggataccaggtgctgtcttcagaagtactttgcatatgaagtctcccagtccacccgtgaggtctgtgtactttgttatccccattttataggtgaggaaatagcacaagaagggtggacacttgcccaaggtctcacggcaaataagtgacattgccaggattctagtgtagggggctggctctggggcccacatcgcttggtctgagtctatttctccagctatgaaataggaatcgtaggctgggtgcagtggctcatgcctgtaatttcagcactttgggaggccaaagcaggtggatcgcttgagcccaggagtttgagaccagcctgggcaacagggtaaaaccccatctctacaaaaaaaatcaaaattatccaggtgtggtggtgtgcacctgtagtcctagctatttgggatgctgaggtgggaagattgtttgagcccaggaggtcgaggctgcagtgagccatgatcacaccactgtactccagcctgggcgacagagtgagaccttgttttaaaaaaaaaagcaataataacatctacctttataagattgaggttaaaggagcttagggagagtagccagcagtgtgcctggcgccactgacagctctgaaaagttacttccctgcctttctcttcattatacccctgaagtgcacaagggccttgttcttgaagggttttcctgctgagtgcatgcgtgttacattctcattatagagaggaatctggtaatctggtaaggagtgggttctttctctatgttgagttgagcagagagaaaaagggctgatgtggtaaacccacacacataccagtctcatcaccttgcagtctcatcataccactcccacagggatccagctaagctgaaaagcaggactgaattctaaaaagtggcttggaaaaaaataataataatacatgaatgccgcttctaaatttaaccttcagatattgtggaaagagcaactctcatctctccaatgacaagacagtttgacagtctctgaaattcaagggaactttctggaatggataattaacagacacatttgagattcctgagcatcctttcaaaatctcaggtattcttttcaaaacataaagaacctccaaattccatgtagaaataaacatccaggccaggcgcagtggctcacacctgtaataccaacactttgggaggcagagcggggaggattgcctgaggccaggggttcaagaccagcttggacaacatggggagacctcatctctacaaaaaataaaaaattagctgggtgtggtggcatgcacctatatagttttaactactcaggaggcagaagcaggaggatcgcttgagcccaggagttagaggctgcagtgagctatgcagtgagctgcagttcaagaccagcctgtgtgacagagtgagaccctatctctaaaaaaaaaaaaaaaaaaaaaaaaaaaaaaaaaaaaaaaaaaaaaaaaaaaaaattcaaaagataatttcggtccagacccagtggctcatgcctgtaatcccagcactttgaggccaaggcaagtggatcacctgaggtcagaagttcgagaccagcctggccaacatggcgaaaccctgtctctactaaaaatacaaaaattagccgggtgtggcggtgcactcctgtagtcccagctacctgagaggctgagacaggagaatcgcttgaacccaggaggcagaggttgaagtgaggtaagatcaagccaccgcactccagcctgggcaacagagtgatactcctcaaaaacaaacaaacaaaaaagataattttgaattagagttgattttcacccatcagactggagaatttaagaaagattgataatattgagtattggctagggtgtgaggaaatgccccaggattaactactgggagaaatgcatattcaggcagtctttttttggagaacaatttggcagcatctatcaaaattgtaagtaagcatgccttttgataattttattgctaaataccatcttacaaaaataccctcctaaatacacacagttgaatgtttatcatagtgaaaaaaacgaaaatgccctaaatgcctttgacttgcctttggttaaatagatgatactgtatctacattatgaacaaccataaagaaattaaaaataactcagtagatttctatatatcgatatgataattttctgtgctaagtatggtaagtttctagcatttagctagaaaagtaatcctgaaaagaatcatagcaaattgctaacagtggccagctgcagggagaaggggatggggccagtgcaaggaactttcccttttattctcctatgtacagcattgttggcatttttttacagccctgtaatttgtatgatttaaataaaagaaacatatttgagggactgaggcaggaggatcacctgagcccaggagtttgagaccagcctgggcaacttggggagactctgtctctacaaaaaaaaattttttttttgagatgaagtttcactcttgttgcccagattggagtgcaatggcgccatcttgtctcactgcaacctccgcctcccaagttcaagcaattctcctgcctcagcctctcgagtagctgtgattacatttgctcgccaccacatccaactaatttttgtatttttagtagagaacggggtttcaccacattggccaggcaggtcttgaacttctgacctcaggtgatcacccgcctcagcctcccaaagtgttgggattacagttgtgaaccactgtgcccggcccaaaaaatttttttaaattagccaggtgcaatggtgtacacctttagtcccagctatttgggaggccgaggtgagaggatgggttggatccaggagttggaggttaccgtaaggtatgattgcaccactgccctccaacctgggtgacagagcaagaccctgtctctaaaaaaataaattaaataaagaaacaaaggagaaaacagtaatcctgaagcttgtgagcaagctgctttatatcagcaaggttcatgctttaccttatgtaagaaaagttactaaagttatttctggctctactattctacattacttcttcttttttaaaaagcattttcttttctttgtttctttttctttttgaggcagggtcatgctctgtcacctaggctggagtgcagtggcactatctcagctcactgcggcctggacctcccagactcaggtggacctcccagactcaggtggtcctcccacttcagcctcccgagtagctgggactgtaggcacatgccaccacatcagcattttcagtgaagcaaaaaaaaaaaaaatttctttttttttttttttttgagacagagtctcactcagtcacccaggctggagtgcagtgtcccgatctccactcactgcaacctctgcctccggggtttgagcaattctggtgccccagcctcctgagtagctgggaccacaggcctgcgccaccacgcccagctaatttttatatttttagtagagatgagattttaccatgttggccaagctggtctcgaactcctggcctcaagtaatccacatgccttggactcccaaagtgctggaatatacaggcatgagccaccccacccggcccaaaaaatgcttttaaactaaaggttttccagcctggcttgagtcaggatcacctgtgaagcttgttcagcatctccgaggattctgaatctatagggctgatctcaaaactattgctgagttgttttattttcatgtattattatttttgtttcaataaatgtgataactgaggctcaaaatgggggagaccttctcaaaaccacaaagcaagtcatcccttgagttagtggctcctcccctcctgccccaggacttagtcaaaaaaaaaaaaaaaaaaaaaaaaaagcttccccctttttacccctcagcctcctccagattggaccagctgtgagcatcacagaattaaacttcaaatattctaaagcagctccgaaaacatttcctcccgtgtgcaggaagagcatcctttgagaaagatgctaaattgccttaattatatttctttcgaaggcaagacgtgcccatctgctttcgtggtagggggtgtggatgcatgctgggcacaaatatacatcaaccccaatacaaacacaattatcaggtctatgcctgcctcataatcttgaaaaagaaggagcacggtgcccatggagtgggtggcaggtcagctccagggctcctcttgaacgtgggttctcattggagcatgctggggccgcggcggacaccgacttttaacaatggggcctggggagtggagagcacagtgctatactgaggagaggctgcaggaatgggggccccagctaggacagtgctggccccatgcaacagcaaggctgtaaaagtcacagcaggctgggcgcggtggctcacgcctgtaatcccagcactttgggaggccgaggcgggcggatcacgaggtcaggagaccgagaccatcctggcttacacggtgaaagtccatctctactaaaaacacaaaaaattagcccggcgtggtggcgggcgcctgtagtcccagctactcgggaggctgagacaggagaatggcgtgaaccccagggggctgagcctgcagtgagccgagatggcgccactgcactccagcctgggcgacagagcgagactccgtctcaaaaaaaaaaaaagtcacagcagcactggaacccaatggaacccactggaactttccaatgaaagttttgggttttcatagaggaaagagtatacgtgctaaatcagacagctctagattcagaccgtggccctggcaagctatgtgactttgggtaagatatgttaccttctctgggatttcctcttccgtaagatggggatagtcatacctacttcattcaggggcggagaggtgatatgtgtacagctcttgaggccaggcataatcaaatgcttaatatttggctcttgttattgttattttaaaattattattatcttttttgaaacaaagtctcactctgtcacccaggctggagtgcagtggcacgatctcggctcactgcaacctccgcctcccaggttcaagtgattctcctgcctcaggcacctaccactgcaccctttttgtatttttaatagagacggggtttcaccatgttggccaggctggtctcgaactcctgacctcaggtgatccacctgcctcggcctcccaaagtgctgggattacaggcgtgagccaccgtgcctgtcctggctcttattattttctgtttgtattcacccatgcattcctgaaaagcctagagttgaccctgcatggtctttctgtctcctttcttgcttcttaggcaggaagaatgcagagaagagatgaggcaatttgtacaaaggacagcaactgtatccatgtcccagaaggaaatctatacaaccttgagtaagtgccttcactctcttgggactcagtttccccatttataacacaaggtctagaggacatttcagctctggtgggcctggtgggccttttggttttgttttaagagacaggtctctttctgtcacccaggctggagtgcagtggcacgatcacggctcactcgaactcctggactcaagagatcctcttgccttagcctcctaagtagctgggattgcaggcgtgagccactgtgcctggctcacctggtgttttatgcataatgacagcaccaagtttgtcctgctgggctgcctccagagctcatgttgagggggtgacagggggacagcagatcagggaggagagaccccctaaacacttgcttatcacttaggagagggtgactgggaccattcagggatgggctgaggatgagagtgattctctagcaaagaaaaagacttggtgtgtgctaggcagtgtccaggaactgatgggaccatcgatgatgatggcagcttctattttttgaaacccactcggtgccaggctatgtagggcattttacatatttaagatcttcatccttgtaacactgctagaaagaagtgaccccttttaaaagatgaggaaacagatttagagaggttaagtccctcagctaaaatggcagaaatgggatttgaacccaggaagatctggcatcaaattcttgtggtttccaaggcaccactagttgggaattgccagagatggaggaagagaagtcagcatgtacacatttcttcccatatgttaagcaggcagacactatgcatatttcatctaaacattgtagcaattttatgagataggtattgatgttcccatttttgtagatgggcaggctgaggttcactgaggctaagaattagaatcgtggaataacagcagggctagttagggattcctaacctgcttttatgctgtggacccctttggcaatctggtaaaacctatggactcctccccagaacatgcttttagttttttattttatttattattattattattattttcagacagggtcttgctctgtcacccaggctggagtgcagtggcagcatcttggctcactgcaacctctgccacccgggctcaagtgatcctctcacctcagcctcctgagtagctgggatcacaagcgtgtgccaccatgcccgactaactttgtatttttagtagagatggggtctcaccatgttattcaggctggtctcgaactcctgagcacaagtgatctgcctgccttggcctcccaaagtgctaggattataggcatgtgccgccgcgcctggcctatttatttttattttttgagacaggttattgctctgtcaccctggctgtagtgtcgtgtcatgaacatagctcactgcagcttcaacctcccaggctcaagggattctcctgcctcagcctcctaagtagctgggactacaggcatgtaccaccgcacctagctagtttttgtgtttttttgtagagacagagtttcccatgttggccaggctggtctcaaactcctaaggtcaagggatcctcctgcctctgcctccaaaagtgctgggattacaggtgtgagccaccatgcccagcctttattttatttttaaaatttcaacttttattttagattcaaggggtacaatgtgttggtttgttacatgtgtatattgggtgatgctgagatttaggatatgagtgatcccatcacccaggtactgagcatagtacccaataggcagtttttcaacccttgccccctcctttcctccctgctctaggagtccccagtctctattgttcccatctttatgtccatgtttacccagtgcttagctgccatttataagtgagaacatttcacatttggttttctgcttctgtgttaatcacttgggataatggcctccagctgcattcatgttgctacaaaagtaatgattttgttctttttttttttagatagagtctccctctgtcacccaggctgaagtgcagtgacacgatcttggctcactgcaacctctgcctcctgggttcaagcgattctcctccctcagcctcctcaagtagctgggactacaggagcgtgtcaccaccccaactaattttttttgtatttttagtagagacgaggtttcaccatgttggccaggctggtcttgaactcttgacctcaggtaatccacctgcctcgtcctcccaaggcgttgggattacaggcgtgagccaccatgccccgccatgttttctttatccagtccattgaagggcatctaggttgattccatgtctttgctattataaatagtactgtgatgaacacacaagtgcatgtgtctctttggtagaataatttattatcctgtgggtacatacccagtaatgggattgctatgttgaatggaacacctcccaggttcaagcaattctcctgtctcagcctcctgagtagctgggactataggtgtgcaccaccatgcctggctaatttttgtatttttttgtagagatggagtttcaccatgttggctaggctggtctggaactccggacctcaagtgatccacccaccttggcctcccaaagtgctgggattacaggcatgagacaccatgcccagtggtagtgttatttttaagaacatgttttaaaatgcataaaataaaatacatagaattgctaaggaaaccaattataaggaaatacagttgtcaaaatatatatatttttaaaccctgacaaatccatgacataataataaaagtgctgctttattaacacattacataacaagatctaacggctggtctaataaccactctaatttcaaagtagtgatgagcataaaatatattttctgtgataaccataaagtgatacaaaaatatctgtgatttttcatgggtatgaaagccacagaaattactaataataagtggtttataacctccattcataattgaagtgatgctaactttcaaatacataatgatgagaacacagatgtaatttttccctatcctggctccaccggaattctctccatggagctattgtgggggttcatgggcccagcttaacacctcttcccagactgtaactgtggtcactagattgaactattttatttatttatttaaattattattatttttattttattttattttattttatttattttttcttagacgaagtcttgctcttgtccccccaggctagagtgtgatggcacgatctcggctcactgcaacctccgcctcccgggttcaagcaattctcctgccttggtccccccaagtagctgggattacgggcgcctgccaccatgcctggctaatttttctatttttagcagagatggggtttcaccatgttggccaggctggtctagaactcctgacctcaggtgatccacccgcttcggcctcccaaagtgcttggattacaggcatgagccaccgagcccggcctattattattatttttttttagtttttgagactgggttttgctctgttgcccaggctggagtgcagtggcgcaatctcagctcgctgcaacctctgcctcctgagttcaagcaagcaattctctcacctcagcctctgaagtagctgggactacaggcgtgtcccaccatgcccagctaatttttttttttttttttttttttttgagagagatcctcacgctgtcacccaggctggagtgcagtggcacgatctcagctcactacaacctttgcctcccaggttcaagggattctcgtgcctcagcctcccaagtggctgggattacaggtgcccgccaccctgcccggctaatttttgtatttttagcagagacggggtttcgccatgttgatcaggctggtcttgaactcctgacctcaggtgatccacccacttcagcctcccaaagtgctgggattacaagcgtgagccactgcgccaggcctatttttgtatttttagtagagacagggtttcaccatgtgggccaggctggtctagaactcctgggctcaggcaatccgcccacctcagcttctcaaagtgctgggattacaggtgtgagccactgcacctggcctgaactattttagaaatgagactttgttgtgctttggtaacctcccgatggcttgtttcattgaagactgaatgaccaaacgtgtgatgctctccagctccactgtacacacctggggaaggaagccttgtgcttggatgacccatactaggggacaggaccagcaagagactgtgataccgatacagggagggtccgcctgggcagaccaattctttctggttcttagtggcttccttggggatcttgagtacgtgacaatgtgctgcccaaagttttctttcttttaataggcaagatctcattctgtcacccaggctgcaatgcagtagcacgatcatagctcactgcagccttaaactcctgggctcaaccccaccctcctgaggagctagtactacaggtgtgcaccactacacctggctaattatttaattttaattttaattttttttgagatggagtctttttctatcacccagtctggagtgcagtggcacaatctcggctcactgcaacctctgcctcctgggttcaagtgattctcctgcctcagcctcccaaggagctggaattacaggcatgtgccatcatgcccagctaaattttgtatttttagtagagacggggtttcgccatgttggccaggctgatctcgaactcttgaccttaagtgatccgctcacctctgcctcccaaagtgctgggattataggattttttttttaagagataggtctctctatattgccccaaatgatctcatattgctggc

SEQ ID NO: 2 - DNA, 33654 bp, Homo sapiens
  misc_feature (1)..(33654): n can be a or g or c or t
gggaatgagtctccatttcttctgcnatntgctgaggttgcctcctcggccagagaacataaaagtgttattcagtaactctctccttttcccttggatgtgggaaggaacgggcgcaccacctttcctccctcttccatttccatgctgcctttttgaagttgcaagagatgctaaaagaaggtccaaccccaagtgagagcctggcctttgcctggaggggtaggggcaggtggcctatatcgccagggtcctggaaaggaacctcttacccactcactcccaattcctatccaactcaagtcactccagtaggctgcttttgagatgcaaatacaaaatgctttcttctccccctctactttttcattgaaatgctttgcatctcagaaaagaggaaaaaatgatgtgtttaagacaggagtgtctcacccgccatcaagcctgataaggaattgttaaattttaattagacaatttgattccttcaagtgccgaactttcagggaaggcagcaccaagacttaagctgagacggaagcataggaaacacgttgctggggtttatgctgcatccagggcttctgtcccccacctttctagctctgtgccccagatgaaatagaaatgaggtggtggggatagagggagggagagagagagagagagagagagagagagagagagagagagagaaagagaggatcagagaaaatcctctctgttctcccaggacctgccagctttcttcctggatcccaggctgtcctaacagattgacccttttcaccccactccatcagcctggggactaagtgttgctacaccttccaagtttctcctgagagaaaatgtcaggtttgggtgtcagcctcaaactgctgagccttaaaggagcttttttcccttccaatacactttataggttggggcgnctgccagttttggggcgtgactgggaaagcagncccttccatacctcatctataccccagaaaggactgcccactccctgtcccctcctctactcctggggggcagtgcagtgggtggcctgagcagggtggcctgcctggggcagcctgggtgggctagcttagccccgggtatctgggctcttccagtacttctcaacttgagagctagctaatttccaaattagggcacgcacagaaaatagtagcatttgtaaggcatatagggtgaatagcagaagctgctgaggttggaagtgcctgcctctacatcccaattgacctaatatttctatgcaccttgatgcattctagacctctgattggggaagttccactctgaagcttctgaagagagccactattatagaaggctggagttaggctagatgttaggaagaacttgacatgcaggggtcctgagcactagaatggatgactctgaaagatatttctgtagatctttgcccttctgagctagggcagagcaaagagtcctggagacagatgaggagatctttggctctttgatgaccagtagcttgcaggtcaccacacaaatgcagtcttctctttctcctggcttccaccagggcagctgagaaagaaaagcctaggaggcctcagaggacccatggaaccttgtctgcaagcatctgcatggtgagggtggaatgaggagcagcaattacccccactttcctgcacagaaaggtgccttaggggagaggcagccacccctggagtgaggcctggcagctacaggattagattgttcatcctccacccaggaaacactgtcaagtagctcaacctgtaacagctggaaaataacagtgtccatgcatgtatttggtgctcactaagttcttgttgactggggattgtgatttggagttttgtaagcttttatgcatgcccaggggccacctgggagaaagatggagatgacatttggagggtggtgagcaagttgatcatattttcacaatcaaaaatcaaaatgcttgttttgataacaatagatttttaaatatactgtgccactttatttgtctcaaaaaaatttctcatgtaagaataatgcaccttgggttatatatttagccantctccaytttaaaaaaagaacaagaaattgggaccatcatggcaaatacaaggtgccatgtgttatgtggctttgcattttccctcaacaagctccagagtggacaaagaactcaccaggcttctttggagccagaaccagcccagcgtaactggagcagcactgagaacctttcctcactccgctaccatgaacacaagctccagccaggatggagaagctactcacttgtgtattcttttgggatgccccttggagcccaacaatctgtgcagcccggagacagaaggacaaaagggagggatgggctggagctgcagcagggaagcctggggaccagcaggttttctcaggcctcttgagaaaataccagcttagaaatgctgggaaggccctgtattgctatccactctacaatgcacttcccmctgcccccaccattttaatgtctctgaaatcagaatgcatttkacaatggatgacatcttggagctctactggggttggatggcattwttttctttcctagtgatatatraaatagtggtgcatcgtcattcgattttgtcttagagtctatgaaatgtggtgattagtatgacagtcatcgrctatagcagctgggctgggagagctgtatcttccaggccagctgctacctcatctcaagggtcacttgagtttgtcctacagccagggcccagggacaggctttgtcttttccttccwttacttcctcccaaggagttggtggctgcatcttgaaatccatctcccttcatctagccacctatttttcaggatgggccataggaggcagtgggcccaggaggacctcagaccctagtgagctgcatttgaacagcaacagtcacttcatcttgctgagccgttggcttgtggtctgattgcttgtttcatttcattcttttgcttggtttgatttttaaagttgacattctcaagggcttacctgtgcctggcacatggccatgagtcatcttacttaatactcacaattgttgttatctctattttgtgtatgggaaaactgggctcaggaaagccaaataatctggctgaggccccaaagttagtaaatggtagagwtgggacttgaacccaggttttttagtcctcaactcgaatgttatactagcacctctcaggctagagtaccttctctgtggcagtggatttgctgatgtctgaattcctgctcagcttgatgtttacatggtgagagccagctaggctgcccctgcttgtgagaggtgaaagccccaagtagataaggtgctgaagacacgatagaaaccaggactgacaccaggtgttaaaagatagccctgtactggccgggtgcggtggctcacgcctgtaatcccagcactctgggaggccgaggcaggcagataatgaggtcaggagatcgagaccatcctgactaacacggtgaaaccccgtctctactaaaaatacaaaaaaattagctggacatggtggcgggcacctgtagtcctggctactcgggaggctgaggcaggagaatggcgtgaatccaggaggcggagcttgcagtgagccgagatagccccactgcactccagcccgggtgacagagcaagactccatctcaataacaaaaaaacccccaaacaagcaaacagacaaaaagatagccctgtaccaaggtacttataggggttacaggtatttaggcatttgccatgttttcccttttttttcctatggtgtaaatatactacgtttgtattagcaacaaaaagcaacgagaacaaccaataataaataatagattagaaaatcaaatgtgtgaggcctttctcagaacttgggtacagggactctcccttaccctgcagaagtaaccagcactccagagaatgaatgctcaccctcaaccccacctgacctgcagtgctcacatttgccatctacattgtactcataattttaaatgctagactagtgcctgtaatcccagcactttgggaggccaaggcaggagtatctcttgaggccaggagtgtgagaccagcctgggcaacatagtgagacccccatctctacaaaacatttttaaaaattagccaggcatcgtggcacatgcctgtatttccagctactcaggaggctgaggtggtaggatcacttgaggccaggagttgaggctgcagtgagctatgattgtgccactgcactccagcctgggtggcaaagtgggattctgtctctattaaaaaaaaattaaatcccagtttacatcctccctcctccaggaagttctccacaaacgccccagccacacacatgtgcatatgctcctgaacatcaactgtagtggacacggtggagtgccacccagatccccctgcaggaccacagccctcatccccaactactgggagtgttggcaactgacagtttttggcaattgcccactgctgaggagagatgcctctttcaaggcctcgctgctttccctgggacagcagaagcttgtgacatctatgagaggggattaaaggccctaccctgttgccctcatttaggcaactggcagggtgccctgtggggtcacctgatgcctttcttgtgacttctccctctggcctgtcctgcttcctcacttctttgcacatcttgatcctgagagcattcccctgtacatttcctgcaggctgatttcctcttcagagtctacttctagggacctgtattagtctgctctggctgctatgacaaaattccacagattgggtggattgaacaacagaaatctgtttcctcatagttctggaggctgggacgtcccagatcaaggtgtcaacagggctgattttgcttaaggcctctctccatggctagcagatggccgccttcttgctgcattcccacttggtctctcctcttgtgcgcgcatccctgatgtctctttttatgtccaaagctcctcttataaggactccagtcagattgggttagggcccaccctaagcgtctcattttaacataatcaactctttaaaggccctgtatcctaatacagtcacattctgaggtactgggggttaggacttcaacatatacattttgtgggttgggggaagagcatagtgcagactgtaacaggacccaacatgcagcacctatggcacccttggggtgccttactttttcatcacttgttaatctggtagcctatgttagcaggtgcctttcaagtgggaaggggttttcttcccagttgcactaacagaacctttgattcagttcagcaaacatctgttgaatacgtactgcccacaaaccagagtcaagagttgggaaagaacaagatagtatggtttttatttgttcattccaagaacaagaggaggaatgagcaagacatgttgtaactcttgtgatgtctacaccccagtgaaaaaaagagactcctaaacaggtgtctcctgcagcactggggtgtgtttcagtgctgcaggaccctgaggagggtgggtgatggctcaggcaggaggatatgggtggattctaagacaggaggattctaggcaagactcactttgaaggatgcatagggactggaaaagcagagggaagggaagtgaacagaagagttttccaaatccctttcttattaaagtagggataataattagacaagatcaagcatgttccaggtagtatggccatagacagggataataattaaaagcctccgagggttgttttgaggataaatgagacagtatatccaaaaaacttagcaaactatcaaaccctttacaagtatatgatgctatcatcagcagcagggagtggggggagaagaagagagcgatggtcaggatgggggcagtaagtgtgcctcacacgcctcaggcccctgacactgctttgcacagtgctttatgcccagcaggttcaataaatgccgattgagtaaatgaatgtcagaagcagccccaggcacctgatcaatacatacatgcaggcctcccgctgtgggctccaaatgggaagagggctcagttggttttgcagaatacagcatcaatatgacatatgcttcattaccttagaacaggaagatttgctgcgtggtggcattccatgtcaccccacatggaattaggcactttgcttttccccgaggctccagagtgatggtggttggtaggacatgatccatattcttctactaaagctggaagagtgggtgtgtcagacagggatgagagtgtagagattgggcagaccctcttccaccatcctctctgacccattgctggaggtgtgccctgttcattgattggacatggcaccacattcatcccaaatggcatcaattggtggttttcaatttgcagtagcaaagtacctggaagtcatgtgctttgtatgaaacgccttggaatgctgataagtttaattctattctgtaaaagaggaagacttttgttagctgaaaagccaactatattatccatgcattatgcttcagtcacaaccaaaactccggctgtcaagttcatcaactcctgatgcctcccaagtctgctcctgagcccctctggtcccttttctctgtgaatatgcgacaccagcctcccagagccccacgcagaaatcctagcattaccctcaactccttatctccctgcttgtcccatacccacttttgccccacacctatccaaggaccaaggaccatgaaattgacttgcagtttagctcctaattctgaacaggtctctccatcctcgtttccactatcttcgttcagatgaccacaatttctaacctaaattacagccaaaacatcactctcctctgagctctcttctacaccatacactttctagaacaccagtccaatagggtcatgtctctgggcccaacctctcagggactctccagctgacaagacacaatctggcctgtaaatgtccctgcagtttaattatcctcaacattactgccataccctacatttttggccaaacagaattgtttgctgtttgatgatttgataccatgtcatattgtctgtctggaacatccccccacccctctcatttagcaggctaagtcctccttctacttcatcccagatgatacccacttcaggaagtctttcccttctgtgggatgagatgcccattctgcagggctttcactccctgcatcctgccaaacctcatcatctcttacctggattcctgctacagcctcccagttggtgtcccgcttccactctgggcccctcctctccgttctccacagtgctgtcagaatcacctattcaaaaggcgaatccgatcatgtggttcctgctgcccttaggatcatgtataaactcctagcatgacttttaaggccctctatgatcttgcctattgcaacctccccagactcaacccttgccaggtccctctgcatcagctatccagaatctctttgaggccctccacctgctgtctacctctctacctctgtgctttgcatatactgcttccaatgtctagaccttctgctgactcctagttctttacctggctaattcctacacatccttcagttgtctgtctgttgaacatcacttcctctagaaagccttccctgaatacctgaactaggttatgtatctctctccctgttccatttcctactccctatcacccttagatgtaattgcttgattaattggctgttgtttctcctagaatgtgagctacagaaccatgtatattttgttcctgcctatatttctagaactatatagaacaatgcttaataaatatttatagaatcaagaatgaatgaatacccatttctgtttcgatgactagaaggtagcaagcctggttaactgaagtgtgtggtgcatgggtcgtgcttaagaagtgtttgttgaatgaataaataaatgatggatcagctctgttcaagctcatcttattatgcatttttaaaaaattgtgtttgctcatttgtcctaccagctgtaaaaacttgctagcaaaagagtggcagaaggcccaacatttttagaaaattcttagccgtaaaccttaaaatcccgagttggaaaattctcattattaaatgccaggagttggtcttcttcctggagacctctgcaagaggccctctcactgacaccttgtgtccatttttcctggccagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtaggtgggccccctgcttgctccagcactttctccagcagggccctgcactggacactggggactctagctccccactggcttttaccaatgagtttcctggtgcttccccaggtggtttttccttcactctgggcttactttttctccttacgaaatgggtagattgtttccttaataatcccaactaccatttgtaaagtgcttactaagtgctgggcctcacgtagggactttaaatatagcattttcctatataaccctcacagcagcttagaggctggcattattgccaccattttgcagttgataaaccaggtggtcagagatgttaagtaactgctgcagcatcacacggctggcaagtccaagctggaattctggcctcagagttagttccttaggtcatattggaaataggagtaaccggcaaggatccccaaggaggggcatgtttatgctcccaggcccctgaaaccttcactcccatctccccacttcagaaatgggtctgctgctttatgctccccatatcccctctccttcccacagcttatcctggggccctgtccaggacctgcaggtagaggctgccatggactgtgctgtagcccttttgttaggaagaattgtgtagtcactcatttcttggcccagctctgcacctcaaaatggggagtaggcagcacagtctagacactgtgcctggtgcattgctgacacagacccagtgaatactcttggtaaccctgggagtccatgctactcttctcactttacaaaataggaaacaggccgagcacggtggctcacgcctgtaatcctagcactttgggaggccgaggcaggcagatcacgaggccaggagttcaagaccagcctggccaacatggtgaaaccccatctctattaaaaatgcaacaattagccaggtgtggtggcatatgcctgtaatcccagatacttgggaggctgaggcaggagaattgcttgaatctgggaggcagaggttgcagtgagctgagatcataccattgcactccagcctgggcgacagagcaagactctgtctcaaaaaaaaaataaaattaaattaaaacaaaaacaaaataggaaatagaagctttggagaagttgcctttcttcacccaggtcacagggagaaatgctgatggagaaatcccaaagcagatattattctccaggaagacaccttcagagccagcgagcagatgtgggagcatttagggatcatcggtgggcacaagctggatcctgcccctttctctgcccagctttctaggatggcatgtccaggactcaagcaatctgggagtctaggtgcatgggaaggcatgggatcaggcatttaggaaagtgccggtgctctgcccactgaaccgttaccttccttccctgagcaggagaggaattgaccagtgcctctgaggccatccctgcctgagagggaaggggttgttgaaagaaaatgagaaagctttgtaggtttaaacaggggagaaatctagatgaggacgctcaggtgaggaggcgagactggtggaaagtggcacacctccctgtccctgggcccccaaggggctcgtgcgcttactgttctcaccaccaccccgggcccaagggagctctgatccatcaccccttggcttcctttactacagccaatgcaggctgcaacttccaaaatgacctgactggatattaaggaagaaccaaaaggaaacacaaatacaaaaccacataaaaatattcagttgacacaggccaggagtccaattccccctggatccatgtttatgagggsccctagactctgtccatgccttcttaattcttacgcttgatcccctccttcttccctcttccaaaggagggtcctaggtctcagaccatagggraaaatggtagagcaaacaaactctctgtgcctcagtttcctcatctggaaaatgcagataataatagtatcatacatcatagcattgttgaggatcgaattagttgatgtatgaaaagtgattagaatgatacatggctcacagtgagcactgtgtaaatgtcagccatggcgatgatgataaagatgaagatgacaatagacatccagcactgctccacatagggaggactctgtctttcctattcaccgcccccttgggataaagaggaaggagagagtactccctatgttcgccccaggcaagtaggtaccctcttcccagaggaacgtgtgttctacccagggctctgggcatggcttctgttagcttccagaccccctgtccctacccaggcacttgggtagggacaaatccatacatgggctatagggggttcccaggaatctgtaagtctttcgaacagatcctttaaacatgcttgttcaccttattttaaaggtgaagctgagtgtgtcaggggaggagggtgcagaagccattctcagagggcagggstcagacttccccacagcatctcatcaaactagagctgggcactgagccttgtttaatggacattatctcagcgatttgttgtccctagagaacgtcccaggtgacgtctcaccctgccctgatgtcctatcagcgcaccctcacaaccacgcactgtcccctctctaatacatgctcttgtcatccagattttttcctcctgttccatttccagcaatgccatgccaacatctgtctggcttaggactaagtgtgcgttatttatcttgctaagtgtatgaaaagcacgctggttttggagtgagaggaccaggttcaattcccagctccattcctttccatctagatgctattggaagagttcctgatgtccacatgtgccattccatgtcagcctcacaaccctttaaggagcggcttcctgtgtgcatttattttctagatgaggagattgagaagatgaacaaatatctcagagttattcattcactcagtcagcactgactgagcactccgaggtcttaccccaatccttgtgcaatgggctaaggcactgacctgctggggatgcagaggttgacagggcccctacagtgcagtctgctgccggtgacagtggagcagtccccataggaggtgtggggcataaggcccagcatgagggtgtcagggaagactttcagggagaaatgatgctttcggaaaaattatgcaggagagggagggagacacttgcattctcaagaaagccacccagcagggagagggagtagctgbctggaggtatggaggagaggtggttatgtcatttgcctctgaggcttactgtctgcattctaagatataagcatcaagtgtttggaacagtgcctgacacatggtaagtccttagtattattacagttattaggacttagctgagccagctcagggcctgtactgcaggtctcagctttatgtgagcaagagcattaaggaatgatgcctggatgcctgggggtgtgaagaaaagagccttgggttcgactagggaacctggggccactccttcctctgctactaaatcaccaagtgatcttgttctgttttcttctctgaccctccctagttttgtccacccttgaaataatcatctttccttttcacatttcatgcttaccaagtacttgtcacctaattatctcctctcttgataagctagatggtyccttccagggcagcttagtagagagcatgggatgtgatgtttcagattccagctctgctgcacacctgccaggtgaacttggccacgttacatggcctctctgggcttcagttccctcacctatgagtgggataagcaagcccttcttgtaaaagttttaagaacaatacatgagataaagtgcaatgcccacagtagatgtcttatggacagtggccaccaatgcattttcttgagtctttaagttagcagccctaactactctccaaggcagcttcccctgggaccacagaggaaatctctttgttattctgggctccagataggccagtgcaaggagagatttaagatgtccatgaaggcaggactgtgtctgaattgctcacaactgtgtccaggagcctaggatagaggctggaacacagtaatttgctcaataagtgtttgtaggaaagaagggatggcagaaaggagggaaggagaggaggaagacagggaggaaggccctgatatctcaacctagatccccagtggctcccaagtactggtcccaggaggggcttcaaagtgggatgtaaatcaggtagtgctggttttctgcccagtggtaccctaaaggcacactctgtttatgccaggaaagccctttacgtaccctcctgcctccacccgcaaaatgggcatccagctgttagctcctgccaacctggtcagcgcagcagcacagggagctgggagagaggccctggtacggggcccccatgtgagctcccaggaagacagtgccgcatgagaggcttgctgtgttctggggcaagctgctgtttctgccacacaggatgacagcagctgcattgcccatcattgagcttaaatgccagcatttggttggatgcctaggagcagaagagagggcagtgatcagaatgagcaaggttataattagccctgctgccaagctacacggtgacacgaagtctgccctactccgggttgcgggagggggctttcacgctcctctctgctcctattggtagaagccaaaggtgcatctccctgtcctgactccatcctcagggaactggggctgctccaagcttccagagcgctagggacagctgccgtgagtgtgtgcctgcgtaatctgccatgagtgtgtgcctgtgtgtgaatgggtgttacgtggggaggggggagggcggtgcgttcatgtgtgtgtgtgtgtgtgtgtgtgtgtgtgtgcctgtgtgcatgtgtgtatgagaaacgtgctcctgccaggagctagactcttcatttcccatcctaccaaggacacagctcatcacagcaacccctccgcctaccccagcccactcgggcgttcctcgagcactagcccagatctgccttctttcgttctctgggtataagaaggagcagaaatttcttctctgtttcatggcgagtcttcatcttcaccattattcttaacagatcattttggggccccgactctgccatgtactgggccaaccactgaagggcttgcctggctcagccagagcctgggccagagcaggactaggaactgagcccacaggccttggggaaatcactgcactttttggaggcttggtttcctcattggttctcagtttcctcacgggcattctaaaattttacctggaatctaatttacagaggtatggctgagtgataacacagggaagccagtgccattgggaagagtttaacgtgatacacaatgtccctagaaaccagatgctcagcccgccatgtggggtgcagggggaagcagcaggcttgggtcaagagtcagaatcaggggtgagggaaagcccaggctacagccctgttggggctcccacaggaagtgcaaggcagggcagattaagcaacctagggttggctagtctcaacatcctggcaggctttgggctacaggggtgatctctagttgtctctctggtacctgagataatttgggacaggggaaatattgtcttggtgtgtgtgtgaattagagaaacggggtggctggcttgcatttgagaggcatccttccaagtcagtcattttctgtcttcaggaattaactagctctgggacggtaagtctctctctgggtctgtgaggctcccaagtgccagaatatcaggatcagagaagatagaaaaatatagtcaatataggtgtctctgtgatgaatgggtgccaaatacacaaatacagaatctaagaaaacacatggggttaacaaaggttgcagagattaaggtttaaagttgaggccctccgtataagttggctgtctttcttctagcacagtaattggcaataagtggtcttatgtatctgggagaagataagtgagggscaagggcctcagacacccatgggcatcactccccacaccccagctggatgcccacaagacttgcagctgcctcctgagtctgtggcctcatcggctccaggagtaccaggctacaggacctaagctaatctcccactcctgctgtccatccattatgttgctttgtccccaggtggtaggtgagtggaaattcagcaggattcactgtgacatcttcgtcactctggacgtcatgatgtgcacggcgagcatcctgaacttgtgtgccatcagcatcgacaggtgagcccagccagggtggaagaggttgctctggccactcaccattctggctgccccagcttttctcaaagccaggctgtgtctcgtgtctgtgatgctgtgcagctggctgtgtgcatgggggtccatgtgtttgtgtgtatgggtgtgcatgtgtgtatgtttggctaggagagcacacactccctaaagagaagccatctatggacagtgatgctgccaagcatactcagacagtgactggtacaaaaggggaaagattgtgattctgacggaagccctagctctagcacatcttactcatgtgaccttgggcagatgatggacgttttcagagctctcagtttcattttctagaaacctacattgatgacacctgtggcctggagctgcagtatctggttatgcattttatgtccttggcacatggccaagggtcccaggccatgcacaccttgctaagctgcatccttggagccttccactaatttgcacagatatgaccctacctccctcacaccattcttggaagatgaaataagttaatgratgagaagggctgggtaaaaaaaaaaatgtgatgaaggattgtagacaattgccattgttatttcctgctaactaaggcttaaatcttgctctgaggggcctggctaacaatttatcaacaaacatttccaccttatgggctcaacacaagtgcagtgggtacagtttcaaaagtgcaaagatgcaggccatggttcttcgaaaagcctgaaatccaggtgggsccggttagaataatagatatgagctaagagtaaacaatagctcaccagtgattaagtgtttgggctttgccaactgtcctcgccatctgagtttggggacaggagggagggttgagttagcatctcaggcaagcttcatagaggtgtgtcaactgtttagccccaaggagtgagggtgtctctggtgtgtgcatattgtggagtgaggggtccctgggcctgcaccccagattcagggtcccccgcccttgcaggtacacagctgtggccatgcccatgctgtacaatacgcgctacagctccaagcgccgggtcaccgtcatgatctccatcgtctgggtcctgtccttcaccatctcctgcccactcctcttcggactcaataacgcaggtacattctgcttttgtttgcctgaggctcagctggccctgggcccctgctccctcgaagggccctgagggagcagagtccctggcacagatatgggtggaggcccaatggaggcctatcactaccctggactgagtccaggctacgtgtcctgggccaagccccactcaagagttgttctaggttggcagagaggaaacagtggcccaggacacaacggagttggatgaggagtggggataatggttcaggggtatgtctgggaattttcaactttggttattaagttgaagaggcaagacgtgcttcacaaactgcatgtgtgtatatgtgcacatgaacatgggatggcggaggctactgggcttccatccactcctgacaatagtatttttgtaatgaagctgcagccctcagggcctgctaaacccaggtcaggccttagccacctggagcaggaaagctagaagtgaataggaggtaagctgaccacatgcccatttctgtgggaggcagagtcaggagtgtctcagacagaggcaacacaaagcccccgccagcgtggaacttcctctcatcagggaggcaagagcgatggcaggtgtgcacctctggagatcgtaacaacaaaagatacagtgaagccttgaaaatgtgtggttcggtatctattataagcatcacaggaattctaagtgggaataacgctaaacatacttggattaatgcacaaggccctgagatagaggacaggcgtctggtagacctagaagggcacgcaaacatatgaaacacataggaacacaagtgagttcaacagacagagccaagttatcttgctgcaaacattaaaaggtggccaacctctcccaatacacaggtcagactaaaaagatggtttactcttttaaaagttttcttgtgtcattctttctggatacatcggcttcacttgttatgcccagacatggcaaaactaatgaccaagtaatgagggaatagtaatggaaagacttgggagcagtccatcatcacagcttaactttttgctcacaaccgtgtttttaatactctggtatctgctgtgcgtttgtgtatatctaagatgaccaggcagccttaaacatctagttgcgttcatattctctgtaaaatcgctccttgttcctggaaggacatgaatgggctcttgtggaattatggccggtgggcccgctgactccctgcctgcccgggctctccctcccccagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggtctcaagacaccccccaaccaactccaagggtccccacctaaccattaccaagagggctcatcttatgctcaggtgggggcttgggaaacctcagcaagggttaggtctaggctaaaggaattcccaggagccgggcaagggcagattttgaaggcatggacctcagtggagcaatctgagtctccaggagagggaggcagggtccatgacactaaataacaaggggaagtctttgcttggagatctttgtgactgaaggcggcaactcttgcttggtacccccgtgggctcctctcttcctcttttctggtttctctgtctcactttagctccctctgactcctccatcctctttcctttaccttccgtactctctgtcctcccctccaaacacacatcacttttcctgacttcctctccactatgtctctcctgtgcttctctttcatttcccccctgatgtcttgtgaattctccccttcactcagtccttcacaaggaagacagtgtgtgggcacagaaggaacaagagctcttgggctagacgcatcaggttcagatcctgtcactgacactttttttgctgagtgaccttaggcaagtttcttaccttctatgagcctgtttcctcatctgttaaatgggaatcaaaataccagcctcacagggtggtcttgaggattccacgtgagaagggacgtgagtgtgcctagcacagtgccaggcccttagcaggtgctccataaaaaaccaggtccttgtgttcctcgtcatacccaccactttttgtctcattccagccttcccccttgccccaactgcctcctctggcccccacccttctgatctctgagcccttctgcccagtctgagttccatggactgctgcactttgggtttctgtcccccctccatccccaccactttgtgtgacccatgtgctggctcactccacagggcaactgtactcaccccgaggacatgaaactctgcaccgttatcatgaagtctaatgggagtttcccagtgaacaggcggagagtggtaagtgctcaggccaggacccagagccaggtctttctgcccctagggaagcccactggccatggttctgagacctcagaagctggccaatgggagaagcaccccagaaacccccaccttgcctcagctgaaggcagactcaccgtgcacacctccaagcaggcatgaagtgagacacctcggttctgcaaggcatggatgtgtacgagaaaatggttggccataccaacgtaataaaaatgataataatggctattcacatttctcaaacatctaccatatccctattatctcatcaaatcctcaccacgaccccgggaggtaagtctctttgatcgaaccgatcttcagttgacagaagaggaaacaggctcagaaagattaggcaactcacccgtctcaagagttggtgacactaagcccagacctgtgtgactctgaaatccacacctgtgttctttccactgacatgagctgccttatggatgggcaggttctggggtaggacgagcagagcagctgcggggactggtggcggassagtttgtgtacatagagccctcaggtgcggaagcmmagcagaccccagcctctgccaggtggtagctgtaccaacatgcaagcagcaggcattccatcctccagagggatggagaacagggccagagaacccacagagggccgcatacaaaatccaggtctggtgtcctgccttcacctgcactgcaagggcaggactctaagaagctgtttatgaggcaggtgccaaaacagagcctcagagtcagggccaaggcagcagcccagtcatgccacctaggcacatagtgaggctgcactttagaagttcagactaacacctccaaggcctcaaacaagagaacctatggaagaacccaggaggccatgagtggatccatgccagggctctctaggtcaccccagcagggaagatgtggggccccaggggtcagccttttggcacctagattagtctatccaggagaatatggagcccacgtgtgtacgcaggtgcaggtacccatgaagtgggagcactgggccccttgtttgacagggagaacaggggtgggcaccctctttgcagtcggaacatgagtttctggaggcagggccaaacatcatcagctcttgaccccagcagccactgtggcacctggcactttgctaacagtaagtgttgctcaggccatgagagacaagtccctggtattcagccctggcagaacagaagtggggtattgaggctgcatgaggattgccatgggaaaaaggacaggggcaatcctgcaggggctgccatgggtcctgggtcccatgcctcagtgacatccttgcctccctggcagggtgaccctgtggtgtttgcaggagtcttcagagggtgaaagggaggggccagtgagatgggtggctgatgcctgggaacttgtccggctttacccagagccctctgcctctggtgcaggaggctgcccggcgagcccaggagctggagatggagatgctctccagcaccagcccacccgagaggacccggtacagccccatcccacccagccaccaccagctgactctccccgacccgtcccaccayggtctccacagcactccygacagccccgccaaaccagagaagaatgggcatgccaaagaccaccccaagattgccaagatctttgagatccagaccatgcccaatggcaaarcccggacctccctcaagaccatgagccgtaggaagctctcccagcagaaggagaagaaagccactcagatgctcgccattgttctcggtgagtcggccctggctgctggccacagcccggtctgtgaaaggtcccattccctgccatgtccttcctaaccatggctgcaggatcatggggntagcatttcctcaggcacaggccaagcccattgacatacagacagccctattaggtagattctgtaattgtgctcactttacagatgggcaaacagagttttagagtggttataaaacctgaccaagaggacccatggtttgaactcaggcagtctgactccagagtctggaaatttgactagtctattatgctggcaccagaacctcatgggcctatgtgcccaggaaagtcactttctctctctaggctcctcatctgtagagtaggttacttctcagggctgtcatggcaatcagttggtgaaagcattttctaaactacaaagcactatccatatgtaagtgtcactattatggtttttattactatggctctttttgaggaattgggaaattcagttcacttgcactcaaacaagactccatgggaccagagctgtgaaaaataaatgagatgttggggagtggcccccaatctaaatgaaaagcatccctgtccataaagcaggatttgtcaatagaagtctagaaaaccacatagtaaaggtcaaaacccaaaaggtggactgatatgggtggctgggaaaaggagggtcccctgtgactctgtcttccaggaagcacctgaatcccctttgactgccggatgatcctggggcgtgagtggggcagctgttacctcccttcttttatgaatgagagcacttgatcccatgggactactcacgatcatgggactgggatatggtaggctaggattggagccaggccctctgctgcagtccataggggctcagctctgtggccctactcttcccagacaccattcagtctccagggatgcctcctgtgcctgtagggcattccatgtacacattgcttccaggctaaatatcacacctgtgcaggaggtcctggaaaaagtgcacctgagttaggtgagcagagcaggtaggtggctgccctgggtgagagggcatggttcaaggcagggactagcaagggtgtacccaccaagactggggaaattgggggaaggctagagcgagatcatctggggacctggatagaccggagttcagctctcagccttgctgctcaagagtgaatgaccttgggtatattgcagagctgttgtgagtgtttttcttatgtaccttatgaggatgctataagaactaagtggcatgggtctatagccctcaaagatgacctggaagtagagtagatatttccatttctactggctaacctgggtcagaatggtaaccttttggatttattgttaaaccatgtacccttttctaggaggtgggaaagggacaaatgagggaagtgaggctgaaggagctgaagggatacttcactgcaaatgggtgtcagaggcaggtctaggatccaagaccgggactcctcctacagtgcttctgtggctacagcccaccgtcttggcatacgagccagggcgcactgggtgtgggtgttcccagccgtgcctccccggctctggggaccagcctgaccatgccctctcccccaggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccrgccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccagscaacttcagtcctgggagacccatgtaaataccagactgcaggttggaccccagagattcccaagscaaaaaccttagctccctcccrcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtagaaggcagcagtgccacttctgtgcttggcattcaagtataggaagacccctgtgtctgcagggtctaacccaagggaagccaggttgcccccactgntccacctcccctgttgcagctcctgcttcctctgaaggactcatcctttgccctcttacccaccagggcagagaaggctctgtggaaaaggtggccttggatgcactggcattgcctgtgtctgcctatgtcccttgncctgtcttctgtcccatgtcaggatccccttcctctagggcaggctgggagaagcagggaaggccctgaccactgcggcctggacagttctccctcctctcagcttccagggcggtcccaagctccaagccttccgggggaaaaacttggtactgccccaacaacagaaacttggctttctacaaatgaagcgtaaatcancccagtgagggaggaatattcttaccaccttgagaataaccgcagtgatgacaaacaaggtgccagcacccacgggctcaggcgctggggagctgtcagggcgtaatttgcatgctaaatacaatatttttagcaccaaagtttggagcacttaacttgccctgaacagttaattatggactttgatcttctccttaaacctaaaggtagcactaagccctgggagaggctcctgtccccaggagcaccctgattctggaaagtgagcaaaacaggcccctagtctaactcggactgggtcataacaccaaggacccagtgaccatctcctctggaaagcatcaggtccccaaggggtctagaagccccagggacccaacccatccccattgnacacataccatgctcaatgtctgtgaaagatcttggcctggatggacgcttaaaagtatatcccacaattaggaatcttatgagggtatacaggcttatcagatgtgangattggaagagatgacaaagagaagcagaggaaagaagaaggaagggagggagggagagagggagggacggaagggagacccagagcagagtgagaagagcattgacagggagcagaggggaagagggcagngcaggggcggnaggcggtgcaggggaaagttgcccacagttgtcacgaggcttcatgtctttctgccagacagcagattgacagctagagtgggcaggggagggctgggctccaccctctccccccctcagcacttcaggggcaaagaaatgggaggagtaggacccgacacgacacgggaacacaaggtggaagggggtggcccaggctctgactctctccagagaggtcctgacgatggtctcttgctcttgacgacaggatggaaaggaagcctccagttttcactcctctttgcctttccctaggtttctgtctgtgnctgtgccgctctgtagagtgccttttaatcaaaggatcattcatttgcctttacagcaggtagagtctgcacccttgtcccctgccctgccctttcctagagcacagcccaggatcaccacctaagggccaggcacagtggggcactttgcatatgtcgtcttagatgggaatggaaatcacacagtcacaaaggagcagacccagaggtaactgcaagtcttcaaggcttcaggccctcgttcttcctcctgggtcctgaaggactctcaggtggccctgggctggggaaagttcctgggaattagaagacgagttgtctagcagactaagaagttgcattgcttcgtccacccatctgctgtcttccttagggaatatttattgagcaattcttgtgctccaggccctgggtgaggtgctgggattaaaacagtcaacaatggtcaccggccctgccttcgtgagtccagtctagtgggatcacaacaaagtgagactctgcttcccccaaggcagagcccagggacggcagacacagcacaggcacatgctgcgcacttccaaggcacctttctcctagatgacgtgtgacacagcagccctggtgtgccttggccttgaacaattaggtagggcaccagtaggggcaggaagactgacagcatggaccactacttcctgcgtggctggggcaggaggccctgaaagaagcagctacagatcctgctttccaggtggtctcaccacaggcacagccagttgcctgcaactaagaacactgaagcccggcctgctttgggggcatttccagcatcccctcctatctggaatctcttcccaaaacatccctgtccccagggactgcatagctcctttcacctggtgggccagccctctctgtgtgccactggctccctgccntcctgggtcccaggacctgggctgagtgtgcaactttagcttccatctgcacagggccctctctgcgggctttgccctccctgatggcatgttcttctcctaacccactggagtgaggggcattcttatcgtctccacttacagaagagaagcctctcatacagaaagggtagtttccctttcaggctaaatcttctgattttacctcttatagtgtccattccagccctggcttctgaccagcgtgagtgcagctgaaagaggctaggatggtcagaagattttttttaaaagagaaagaatacttgttttggaccagacctgtagggtaacagcctggggtgcatgtaaacagaacttgggcctctccagggagcaagtgggtaggtggtgggggcagagcagttattagtggggcttcaggagtgagagggccagtctgggcatcggtgtgccagagggtcctctcctgccaaccaggtggacctggctctgctacaytagtttytgggggtgcaggggaagcaggaagctggtggggggagagtggaccatctttggagtagggagacctatcctggctttgtggggttagaggggacgtgggaagggggtacctggtaggaacaccttcctgcttcctctctcatcacagttctgctacttgscaaataaggcctcattggagcagaggcccagagaggcgcaggagagggaatggcaaggagggatgtggagagacctttcactcacagctggagccagagacaaccccaagttcataggaagtgctgtgtgaccgctccacttgcatgtggctttctcccactttcacatctggcttctgacacccagagctgcgtgtgaggccagggagagggggccacctccctggcaccccaggnccaggggtgtcagagctgcacaggaagtgaaggaatcctatcaggaaggggtgaggtggtcccattccaggaatgggagggcaggcctgggccaccgactgtgcagagacaaagctggccatccataccttctggattttcctgatcaggcccaactccaaacactctgtcctgttncccccaggagtgtgtaagttgggagggtcatttgggggccaggctctgtnctgcgtccagggcaggggaaagcatgggaggggcaagagaggcaggacactcccctggagaagacaagtcccttgttggttttctcccagggctgacagcacagacctgacccaggctctgggtagaggaggcatgactggctagtggggctagcctttgccctataactgacctgatccgtaggtgtttcccaggggncctcggtgtctcagcctacaccaaggcactgaaggagagtctccctttcctttcctgaatgtatcggcaaagaaaaatgggcagaggcttctaagagcatccttgcaaaaggctcccaagggatgggctgtctatgtcttattgcaagagaaacattcctctgatttaggaggaactgcaagaaatgaatgtgttgagtgcttattaactgccttgccctctgactggcagggcatcaggtttaattcttacaatgacccagagataaacattattgctgctagatgataccatcaagctaagatagttttagcaacctgagatctncaactanaaatggctcagcgaagacatgcacccaggtctgcctgcccgagtccactagacccagccacccgcagtttccctcnccgacctcagcccagcctggttcctgccctctgtccctaacattaccttacaccaaaaacccctgctgcctgtaatccgattcagcaagcagggaggaagcaccctctgagctccagacaccagggcactaagagaggactggatggaacacagaaacaagtagggcgtggtgatgtggcatgcaatgagcaaggaaggtggacccaagagaagctctgtcaacctcactgcactgggcatcgggacagaatgcccctgaggaaggagtgagcatctccagggggcaagcagagacgtcttcgtgggcaggaacaggcaagttggatctggaagggcagatcggatcccggcaggcaccggtggaagaagaggacacagcattagcaggaagaagggcctggggttggtgggtgctgcagcaatgctggctgggtttcagagtgggctnggagccctaactcactggaggccctcacttattggtgagatgtgttctataggtaagcacagggggnatcaggaaaggaatgttcctgaacacaagatcaccttggagctgycatctgtgtgttcaggaagtcctggggntccctccttcctctcctwcttgccacaggcctggagccctgtttcctccccttctcagacctgcccattatcattctcttcctggggctcagtgncctcaggttggctttggagagggacacggtctccccagtctcctcatttcccgtaggccttttcagccgngaggagttttctgctgatccctgcagtcctgcctcccttcaagacagtcgcggcagnctgtttaggcctctgccctacactttgntgtggagacagnttgmcaagnctccctgytcctttaggattcccagcctcataactggagggttccaccaacagcttccatctcccagcacaagaagggatccaggtggaacaatctcagctatctgcatctctggggatggctaggctaggggatgccatctcccaggacccaggaatgtcctccttgaggtgggctggattggatcaagcacagtttaggatgggaaaggaggctgtcatatgagtaaagggtaatgaaggggttacctgagtaacactggtgagaaaggacaggaacagaaagctggaagaggagagaagtgatggtgagtaccaatgtgtgctggagataggcgtgatctaattgcatccccaagataacaaatctgttataaatacagatgccgaaatctggaaagtctgggtaaagtccacagctagcaaaaggcagaattgggatgccaagccaggtgtgccttgctctgaagccccatagggcagggtccccatctcccaccactgtcccccatttcctcatcctgcacacagatgaagaggtctttaggatgcatggngtgttctgattgnatcctccttctgctgggaaactgacaggaggtcccaactgtccctcatcctgcatgggagntcccaaatcagaatgaacattaaggtctctccattgaatacgcacctcccacacctgcacctcaacagnactgtccctccttcagntgggcactctctgctccccatagcagntgtgtntntttcacattccccagccaatcctctccccttntatccagtccaggctttgctcctcttntgctctgtccttgtcctcattcaacatatgcatcttccaggaaaatctttcctgatcgaccccacgcaacatggcaactgcctggcggaggcaagacaggtatagactgtgctgtccactgtccccatgtggctagatacccacatttaatttattgaaataaaaaaacttgattccttggttacactagccacatttcaagtgctcaggagcatctgtggctggcggctatcctactgagcagtgtggatgtagaaggcttccatcctcccagtgctgatccagaatggctcctaagttaggagtttggataccttcaactaaaatagggtataaagggtgaagtcaggttctccctccaagtggagatgcccagtaagaaattggggagctctttccccccgatccccaatctcccctaaatagggcttgaacttctactggagaccctctccttgggaatttcaagggcctcattcaaacccaattttaaacatccctagggaagctctagaaggcagctgtgttgaaatgggactcaaggtggataccgtgtgtgtttgtgtgtgtgtctgtgtctggctaggagtggcttgatagggcagaagaagacactggtaggcaatgaagggccatgttgtccagggctggcctgaagtcacctctaagagttatctcttagaggaaggaagtggaggaagcaggggtgtgagaatctctgcctagactcaggatcaccagagctgaaatgattcccagggacagtcctgtccacatctccacagcatccctgccattcattggacactaggtgcaaaaggttcatctactttttctaactcttttcttcaaggccttttctcagttctcagctctttccaataggatcagccagccactcttctccctctttggttccctcctgatcactcccactgatggatctgagcacccttggctggaagtatgtagccccagagccttctctggctctcatagagtcctagatttggagttagatctggaagggcagataggatcctggcagacactggtgggagacttcagagtgcaaagagaagcaaacagccctgtgcctcctccacttccctcctcttgctcccgcatgttctgggaaagcttagaactcctaggggaatctttaactacaagttttgcctaaggcttagtctcagttgagactaaagagatttgagcctgggcagctagcaggrggagtagggaagatggaacagtcaggtggagtgtcccctggaaacccagttcgtcagggagtccaaatgggaagaggaaaaaggacttctggacaacccaagctgctgggccaactgatgtctgcaaggttatggctaaggaagggcacagagagaaaccatgggatgctccagtgcttctcagccctgggtgctcttcagaatcacttgggaattgttaatgatgctcacaccaaggtcctaccctggaggttctggtttaattggttgagggtagggccaggcgttggtatattttaaaagccccttgcccaggagattttaacgtgcaggctcggtttataaccatggctctaggacacactccagcttcctgggctcacatctgactttccatggtggccataagctatggcaaggtggtggtaaagggcccagggcccaacaggctggagctgtggtccagaggccaaaggatggaggacacaaagctgctagctctactgaaagggacttgttaattcaacaggattgcacgtgggcaacacattttcntctgccccacctctctcaccccatgctgatgccaggctgctagctgctgtccccatgcccagactggagtgcccagcccaggagaatgacacgcagaggtgggcctgccaatcagcttggctgagagctggccaatagttgggacagatgattccttttctctgacaattgcaaaacatccttgagtgggaggagctgcttctactatgctgaccagcagtgtagggagacagaaggagcaggataggaccccaggaaactcatcctgtccctactccctccaaattgttggctgctctcctggaagctgaactagggccctggtcacataaagttagtgtgaacagctccaagccacaaccagcaggacaggacaggacagctgatgggtggcctgggtgtggtcacaaaagtgagccctgcaagctccatactgagtccagacaacccacccctcaccatcatcaccccctccaactccctctgctgcctctgcctcctgtctttatgtgaactcacacacacatatgaacacacatataccctacattagacaaatcgcatacacacacaagcacaccatactaacgctgctggcctctatatgcaacttggttccacaggccaatccattctctaaacaggagcctcaggagcctcagctttcttgacttcaggagtttgaaatctggctgtaaaactggacacgatgtaggatgaagggtggtgctggggaccagggacctcacatctcacatagggtgacttatccacagttggagtgcacccttcctatatcctcaaaatgccatgagggagacccaggcattgggaccaggggcactggattaggtgtggtcagtgtggcaacagatggctggctgggactttagtgagcagaaacctcctttgaacttttagcttctgccccttgagcaaagcacatgtgtctggctctccccgccccagcctgaggtaaggagccaagagcccaagacgtgagatgggaggatcctgcttccctcaccgctgtgcctccctccatctccagagggggtctgctaggatgcaagggcccctgtgaactcagaagcgagtggcaaaaggtggtaggtgtcttggattggagagggtattcatttggggagcttgcatccccataaaggaggaggaggaggcctggggtctgttggaagtgactggaggatgtgttgaaggcagctctccaagccagagccccttcctcagctacagccacagtgatacctctccccatgcccccatcacccaggttcctgtctcctctcaaggtggaaggggcaggtgaagactgcagacagggaagatgccctgccagaagcccagctgcactttcaccaattcntactccatccaggcggagaggccccaagtagtctaaatttctttctttctttctttttnatatggagtctcgctctgttgcccaggctggagtgcagtggtgcgatctcggctcactgcaacctctgcctcctgggttcaaggaattctcctgcctcagcctccctggtagttgggattacaggcacgtgccaccatacccagctaaattttgtatttttagcagagacagggttttgccatgttggccaggctggcctcaaactcttgatctcaggtgatctgcctgcctcagcctcccaaagtgctgggattacagacgtgagccaccacggctggccaagttgtctaaatttccatctcggctcctggcttagaaccacccagagtggccactgacggctccttgccctctaggaaggacatgatgccctgctttcggctgcggagggccagttgcaggggtgtgcagctcactccatcctggacgtccagctgggcgcctgcctcgaccagcactttgaggatggctgtgttgcccttgagggcggccaggtgggcgggtgtccagcccaccttgttgcgggcgtggacatttgcgtgatgttctakgaggttgatgacactcaggaaggtgctcctctggaccgccaggtggaggggtgtccagcctgactgctctgcagcattggggtcagccccacactgcagcagtgctgacaccaccgcctcctccccgtggcgtgcagctaggtgcaggggagtccagttcacagctccaagagcacccatgtttgcgtggctctctgccagcagatggatgatctccargtggcccttgtangctgctagatgcaggggtgtccaaccctggtgggtgggcagctcaagctggctccgtacctgagcacatcttgcagatcaggtatttgcccctggcagctgcagtgtgcagtgggccatagccgctctggtcaagggcatcagggaccgctccactcttcagcaggtgttggatggccctcactttgccccgctctactgccaggtgcagtggtgttctcaggtttctctgctgancatccaactcagccccctggctggtcagcatcttgaccaggctaacatggccaaagtaggcggccacatggaggggggtcttgccctcagcctcacgcaggttggggtcagcctgacgggagaccagaagccgtgccacattctcaaagttattctgtgcagccagntgaagangggtccacccttcacgttcctgggcatccacacaggccccgtggtccaagaacangcgcgcantgcggtcatccccattctgggctgcaaantgcantggggcc

SEQ ID NO: 3 - DNA, 1773 bp, Homo sapiens
  misc_feature: U07882
ccgaggagcctgcgctgctcctggctcacagcgctccgggcgaggagagcgggcggaccggggggctgggccggtgcgggcggcgaggcaggcggacgaggcgcagagacagcggggcggccggggcgcggcacgcggcgggtcggggccggcctctgccttgccgctcccctcgcgtcggatccccgcgcccaggcagccggtggagagggacgcggcggacgccggcagccatggaaccggccccctccgccggcgccgagctgcagcccccgctcttcgccaacgcctcggacgcctaccctagcgccttccccagcgctggcgccaatgcgtcggggccgccaggaccggggagcgcctcgtccctcgccctggcaatcgccatcaccgcgctctactcggccgtgtgcgccgtggggctgctgggcaacgtgcttgtcatgttcggcatcgtccggtacactaagatgaagacggccaccaacatctacatcttcaacctggccttagccgatgcgctggccaccagcacgctgcctttccagagtgccaagtacctgatggagacgtggcccttcggcgagctgctctgcaaggctgtgctctccatcgactactacaatatgttcaccagcatcttcacgctcaccatgatgagtgttgaccgctacatcgctgtctgccaccctgtcaaggccctggacttccgcacgcctgccaaggccaagctgatcaacatctgtatctgggtcctggcctcaggcgttggcgtgcccatcatggtcatggctgtgacccgtccccgggacggtgcagtggtgtgcatgctccagttccccagccccagctggtactgggacacggtgaccaagatctgcgtgttcctcttcgccttcgtggtgcccatcctcatcatcaccgtgtgctatggcctcatgctgctgcgcctgcgcagtgtgcgcctgctgtcgggctccaaggagaaggaccgcagcctgcggcgcatcacgcgcatggtgctggtggttgtgggcgccttcgtggtgtgttgggcgcccatccacatcttcgtcatcgtctggacgctggtggacatcgaccggcgcgacccgctggtggtggctgcgctgcacctgtgcatcgcgctgggctacgccaatagcagcctcaaccccgtgctctacgctttcctcgacgagaacttcaagcgctgcttccgccagctctgccgcaagccctgcggccgcccagaccccagcagcttcagccggccccgcgaagccacggcccgcgagcgtgtcaccgcctgcaccccgtccgatggtcccggcggtggccgtgccgcctgaccaggccatccggcccccagacgcccctccctagttgtacccggaggccacatgagtcccagtgggaggcgcgagccatgatgtggagtggggccagtagataggtcggagggctttgggaccgccagatggggcctctgtttcggagacgggaccgggccgctagatgggcatggggtgggcctctggtttggggcgaggcagaggacagatcaatggcgcagtgcctctggtctgggtgcccccgtccacggctctaggtggggcgggaaagccagtgactccaggagaggagcgggacctgtggctctacaactgagtccttaaacagggcatctccaggaaggcggggcttcaaccttgagacagcttcggtttctaacttggagccggactttcggagttggggggtccggggccc

SEQ ID NO: 4 - DNA, 16 bp, Artificial sequence
  DRD2-11 forward primer
agcagaggaaggagtg

SEQ ID NO: 5 - DNA, 16 bp, Artificial sequence
  DRD2-11 reverse primer
aatgatgcctggatgc

SEQ ID NO: 6 - DNA, 21 bp, Artificial sequence
  DRD2-11 probe FAM and TAMRA tagged
tccctagtcaaacccaaggct

SEQ ID NO: 7 - DNA, 20 bp, Artificial sequence
  DRD2-11 probe  TET and TAMRA tagged
tccctagtcgaacccaaggc

SEQ ID NO: 8 - DNA, 15 bp, Artificial sequence
  DRD2-24  forward primer
ctgactctccccgac

SEQ ID NO: 9 - DNA, 16 bp, Artificial sequence
  DRD2-24  reverse primer
cttggggtggtctttg

SEQ ID NO: 10 - DNA, 19 bp, Artificial sequence
  DRD2-24 probe  FAM and TAMRA tagged
ccaccacggtctccacggc

SEQ ID NO: 11 - DNA, 19 bp, Artificial sequence
  DRD2-24 probe  VIC and TAMRA tagged
ccaccatggtctccacggc

SEQ ID NO: 12 - DNA, 18 bp, Artificial sequence
  DRD2-25  forward primer
cccattcttctctggttt

SEQ ID NO: 13 - DNA, 15 bp, Artificial sequence
  DRD2-25 reverse primer
ctgactctccccgac

SEQ ID NO: 14 - DNA, 18 bp, Artificial sequence
  DRD2-25 probe  FAM and TAMRA tagged
cggggctgtcaggagtgc

SEQ ID NO: 15 - DNA, 16 bp, Artificial sequence
  DRD2-25 probe  VIC and TAMRA tagged
cggggctgtcgggagt

SEQ ID NO: 16 - DNA, 17 bp, Artificial sequence
  DRD2-35  forward primer
tatggggagaggaactc

SEQ ID NO: 17 - DNA, 18 bp, Artificial sequence
  DRD2-35  reverse primer
gagaagggatacattgca

SEQ ID NO: 18 - DNA, 16 bp, Artificial sequence
  DRD2-35 probe  FAM and TAMRA tagged
agcccaccctgctgcc

SEQ ID NO: 19 - DNA, 18 bp, Artificial sequence
  DRD2-35  TET and TAMRA tagged
agcccacccttctgcctt

SEQ ID NO: 20 - DNA, 16 bp, Artificial sequence
  DRD2-42  forward primer
caacacagccatcctc

SEQ ID NO: 21 - DNA, 16 bp, Artificial sequence
  DRD2-42  reverse primer
tcactccatcctggac

SEQ ID NO: 22 - DNA, 17 bp, Artificial sequence
  DRD2-42 probe  FAM and TAMRA tagged
ctggtcaaggcaggctc

SEQ ID NO: 23 - DNA, 16 bp, Artificial sequence
  DRD2-42 probe  VIC and TAMRA tagged
tggtcgaggcaggcgc

SEQ ID NO: 24 - DNA, 22 bp, Artificial sequence
  HTR1D sequencing primer
ggagactgaggcaggacaatcg

SEQ ID NO: 25 - DNA, 23 bp, Artificial sequence
  HTR1D sequencing primer
ggttttcccaggttcatcttgac

SEQ ID NO: 26 - DNA, 21 bp, Artificial sequence
  HTR1D sequencing primer
cacatcaccctccctgtattc

SEQ ID NO: 27 - DNA, 20 bp, Artificial sequence
  HTR1D sequencing primer
caagatgtctcagggtcctg

SEQ ID NO: 28 - DNA, 22 bp, Artificial sequence
  HTR1D sequencing primer
gactgcttctctgaatcggctg

SEQ ID NO: 29 - DNA, 20 bp, Artificial sequence
  HTR1D sequencing primer
tgatgacggaaaggaccacg

SEQ ID NO: 30 - DNA, 20 bp, Artificial sequence
  HTR1D sequencing primer
gacaaccttgaaggaaggag

SEQ ID NO: 31 - DNA, 20 bp, Artificial sequence
  HTR1D sequencing primer
ggtttccatcttggtaatgc

SEQ ID NO: 32 - DNA, 20 bp, Artificial sequence
  HTR1D sequencing primer
ccgatgaggttacaggacac

SEQ ID NO: 33 - DNA, 23 bp, Artificial sequence
  OPRD1 sequencing primer
gcagtgtcccttcctcagagttg

SEQ ID NO: 34 - DNA, 24 bp, Artificial sequence
  OPRD1 sequencing primer
aaagaaaaatcctaagccaggtgc

SEQ ID NO: 35 - DNA, 19 bp, Artificial sequence
  OPRD1 sequencing primer
tcaagcaatccacctgccc

SEQ ID NO: 36 - DNA, 21 bp, Artificial sequence
  OPRD1 sequencing primer
cccgacaacagaagcaaaagg

SEQ ID NO: 37 - DNA, 20 bp, Artificial sequence
  OPRD1 sequencing primer
agagagggggtttcaccgtg

SEQ ID NO: 38 - DNA, 20 bp, Artificial sequence
  OPRD1 sequencing primer
tggcagacagcgatgtagcg

SEQ ID NO: 39 - DNA, 20 bp, Artificial sequence
  OPRD1 sequencing primer
ggtttccatcttggtaatgc

SEQ ID NO: 40 - DNA, 26 bp, Artificial sequence
  OPRD1 sequencing primer
cattggttgaccttcttctacactcc

SEQ ID NO: 41 - DNA, 26 bp, Artificial sequence
  OPRD1 sequencing primer
ggagtgtagaagaaggtcaaccaatg

SEQ ID NO: 42 - DNA, 24 bp, Artificial sequence
  OPRD1 sequencing primer
ccagatgccagcagtagaagattc

SEQ ID NO: 43 - DNA, 20 bp, Artificial sequence
  OPRD1 sequencing primer
acccagcctcctgttgatgg

SEQ ID NO: 44 - DNA, 24 bp, Artificial sequence
  OPRD1 sequencing primer
cctgacctctctgattctgtttcc

SEQ ID NO: 45 - DNA, 24 bp, Artificial sequence
  OPRD1 sequencing primer
gggactcctacctccatttgactg

SEQ ID NO: 46 - DNA, 23 bp, Artificial sequence
  OPRD1 sequencing primer
ggggtgttgtgggattctgatac

SEQ ID NO: 47 - DNA, 20 bp, Artificial sequence
  HTR1D forward primer
ataaaactgtacacagggaa

SEQ ID NO: 48 - DNA, 24 bp, Artificial sequence
  HTR1D reverse primer
ctttgtagagaaatacattgtaac

SEQ ID NO: 49 - DNA, 25 bp, Artificial sequence
  HTR1D probe  FAM and TAMRA tagged
aaggccatcaggaaaaaaaccaaat

SEQ ID NO: 50 - DNA, 27 bp, Artificial sequence
  HTR1D probe  VIC and TAMRA tagged
taaaggccatcaggaaagaaaccaaat

SEQ ID NO: 51 - DNA, 16 bp, Artificial sequence
  HTR1D forward primer
cggttttcccaggttc

SEQ ID NO: 52 - DNA, 17 bp, Artificial sequence
  HTR1D reverse primer
tcagtgggataggaacc

SEQ ID NO: 53 - DNA, 19 bp, Artificial sequence
  HTR1D probe  FAM and TAMRA tagged
tgacgcatcctaagctact

SEQ ID NO: 54 - DNA, 19 bp, Artificial sequence
  HTR1D probe  TET and TAMRA tagged
acgcatcctgagctactta

SEQ ID NO: 55 - DNA, 20 bp, Artificial sequence
  HTR1D forward primer
gaaagggacaattttctgaa

SEQ ID NO: 56 - DNA, 20 bp, Artificial sequence
  HTR1D reverse primer
ccctcatcaatccaataatc

SEQ ID NO: 57 - DNA, 22 bp, Artificial sequence
  HTR1D probe  FAM and TAMRA tagged
aaactcttcgttaaacacagtg

SEQ ID NO: 58 - DNA, 22 bp, Artificial sequence
  HTR1D probe  TET and TAMRA tagged
aactcttcattaaacacagtgt

SEQ ID NO: 59 - DNA, 18 bp, Artificial sequence
  HTR1D forward primer
gtagattgaccggcttta

SEQ ID NO: 60 - DNA, 16 bp, Artificial sequence
  HTR1D reverse primer
atggtgtcccactcaa

SEQ ID NO: 61 - DNA, 16 bp, Artificial sequence
  HTR1D probe  FAM and MGB tagged
cccacccaccgcaagc

SEQ ID NO: 62 - DNA, 15 bp, Artificial sequence
  HTR1D probe  TET and MGB tagged
cccacccgccgcaag

SEQ ID NO: 63 - DNA, 14 bp, Artificial sequence
  OPRD1 forward primer
ccgctcttcgccaa

SEQ ID NO: 64 - DNA, 14 bp, Artificial sequence
  OPRD1 reverse primer
attgccagggcgag

SEQ ID NO: 65 - DNA, 16 bp, Artificial sequence
  OPRD1 probe  FAM and TAMRA tagged
cgccttccccagcgct

SEQ ID NO: 66 - DNA, 16 bp, Artificial sequence
  OPRD1 probe  TET and TAMRA tagged
cctagcgcctgcccca

SEQ ID NO: 67 - DNA, 16 bp, Artificial sequence
  OPRD1 forward primer
tggctcacacctgtaa

SEQ ID NO: 68 - DNA, 16 bp, Artificial sequence
  OPRD1 reverse primer
acaaagcgagatccca

SEQ ID NO: 69 - DNA, 22 bp, Artificial sequence
  OPRD1 probe  FAM and TAMRA tagged
cacctggggtcaagagtttgag

SEQ ID NO: 70 - DNA, 20 bp, Artificial sequence
  OPRD1 probe  TET and TAMRA tagged
acctggggtcaggagtttga

SEQ ID NO: 71 - DNA, 15 bp, Artificial sequence
  OPRD1 forward primer
tgctcacctcctgtg

SEQ ID NO: 72 - DNA, 17 bp, Artificial sequence
  OPRD1 reverse primer
ccagtctccctcctaag

SEQ ID NO: 73 - DNA, 19 bp, Artificial sequence
  OPRD1 probe  FAM and TAMRA tagged
tgcggattcaatgggttat

SEQ ID NO: 74 - DNA, 17 bp, Artificial sequence
  OPRD1 probe  TET and TAMRA tagged
tgcggattcagtgggtt

SEQ ID NO: 75 - DNA, 15 bp, Artificial sequence
  OPRD1 forward primer
ttccagaccagcctg

SEQ ID NO: 76 - DNA, 15 bp, Artificial sequence
  OPRD1 reverse primer
gactacagacgccca

SEQ ID NO: 77 - DNA, 28 bp, Artificial sequence
  OPRD1 probe  FAM and MGB tagged
cctatctttactaaaaatacaaaaatta

SEQ ID NO: 78 - DNA, 29 bp, Artificial sequence
  OPRD1 probe  VIC and MGB tagged
ccctatctttactaaaagtacaaaaatta

SEQ ID NO: 79 - DNA, 19 bp, Artificial sequence
  OPRD1 forward primer
agatttggtcaccagatag

SEQ ID NO: 80 - DNA, 16 bp, Artificial sequence
  OPRD1 reverse primer
ttgccccttgctagaa

SEQ ID NO: 81 - DNA, 17 bp, Artificial sequence
  OPRD1 probe  FAM and TAMRA tagged
tgtggcctcaactttgg

SEQ ID NO: 82 - DNA, 17 bp, Artificial sequence
  OPRD1 probe  TET and TAMRA tagged
tgtggcctcatctttgg

SEQ ID NO: 83 - DNA, 18 bp, Artificial sequence
  HCRTR1 forward primer
gacccactcatactgttt

SEQ ID NO: 84 - DNA, 21 bp, Artificial sequence
  HCRTR1 reverse primer
agactatgaagatgagtttct

SEQ ID NO: 85 - DNA, 22 bp, Artificial sequence
  HCRTR1 probe  FAM and TAMRA tagged
agataatcgcgccacagatagc

SEQ ID NO: 86 - DNA, 23 bp, Artificial sequence
  HCRTR1 probe  VIC and TAMRA tagged
agataatcacgccacagatagcg

SEQ ID NO: 87 - DNA, 17 bp, Artificial sequence
  HCRTR1 forward primer
gtggaaaccaggatgtc

SEQ ID NO: 88 - DNA, 19 bp, Artificial sequence
  HCRTR1 reverse primer
atacaaactgagagaagcc

SEQ ID NO: 89 - DNA, 21 bp, Artificial sequence
  HCRTR1 probe  FAM and TAMRA tagged
tggggttagtggagtggaagg

SEQ ID NO: 90 - DNA, 19 bp, Artificial sequence
  HCRTR1 probe  VIC and TAMRA tagged
tggggttagtggggtggaa

SEQ ID NO: 91 - DNA, 16 bp, Artificial sequence
  HCRTR1 forward primer
gccacaagtccttgtc

SEQ ID NO: 92 - DNA, 15 bp, Artificial sequence
  HCRTR1 reverse primer
tgagcaccacatgct

SEQ ID NO: 93 - DNA, 19 bp, Artificial sequence
  HCRTR1 probe  FAM and TAMRA tagged
agccgatgctccatctcca

SEQ ID NO: 94 - DNA, 22 bp, Artificial sequence
  HCRTR1 probe  VIC and TAMRA tagged
ccgatgctccgtctccaaaatc

SEQ ID NO: 95 - DNA, 21 bp, Artificial sequence
  HCRTR1 forward primer
ctctttttatcctgtgagttc

SEQ ID NO: 96 - DNA, 22 bp, Artificial sequence
  HCRTR1 reverse primer
tactgttatcttcatcttcttg

SEQ ID NO: 97 - DNA, 23 bp, Artificial sequence
  HCRTR1 probe  FAM and TAMRA tagged
agaaaataggcacaagccttggt

SEQ ID NO: 98 - DNA, 20 bp, Artificial sequence
  HCRTR1 probe  TET and TAMRA tagged
aataggcgcaagccttggtt